(19) United States
(12) Patent Application Publication
(10) Pub. No.: US 2020/0038499 A1
(43) Pub. Date: Feb. 6, 2020

(54) RNA BACTERIAL VACCINES

(71) Applicant: ModernaTX, Inc., Cambridge, MA (US)

(72) Inventors: Elisabeth Narayanan, Cambridge, MA (US); Nadia Cohen, Cambridge, MA (US); Giuseppe Ciaramella, Sudbury, MA (US)

(73) Assignee: ModernaTX, Inc., Cambridge, MA (US)

(21) Appl. No.: 16/496,135

(22) PCT Filed: Mar. 22, 2018
(86) PCT No.: PCT/US2018/023850

§ 371 Date: Sep. 20, 2019

Related U.S. Application Data
(60) Provisional application No. 62/474,811, filed on Mar. 22, 2017.

(51) Int. Cl. A61K 39/09

(52) U.S. Cl. CPC A61K 39/092; A61K 2039/53

(57) ABSTRACT

The disclosure relates to (i) a bacterial vaccine, comprising: at least one RNA polynucleotide having an open reading frame encoding at least one mutated bacterial antigenic polypeptide, wherein the mutated bacterial antigenic polypeptide comprises at least one asparagine (Asn) amino acid substitution; and (ii) a Streptococcal vaccine, comprising: at least one RNA polynucleotide having an open reading frame encoding at least one Streptococcal antigenic polypeptide, such as pneumolysin. Incorporating the RNA in a cationic lipid nanoparticle and a method of inducing an immune response with said vaccine are also disclosed.

### RELATED APPLICATION

This application claims the benefit under 35 U.S.C. § 119(e) of U.S. provisional application No. 62/474,811, filed Mar. 22, 2017, which is incorporated by reference herein in its entirety.

### BACKGROUND

Streptococcus pneumoniae, a gram-positive, catalase-negative, facultative anaerobic bacterium, causes a variety of serious human diseases such as pneumonia, bronchitis, bacterial meningitis, sepsis, otitis media (ear infections), and corneal ulcers. The bacterium typically colonizes the respiratory tract, sinuses, and nasal cavities in healthy carriers, but may become pathogenic in immunosuppressed organisms. In severe cases, pneumococcal diseases may cause hearing loss, brain damage, and death.

Pneumolysin (PLY, AJS15225.1; M17717.1), a putative major virulence factor of Streptococcus pneumoniae, is a 53 kDa pore-forming toxin consisting of 471 amino acids. Marriott et al., Curr Mol Med. 8(6):497-509 (2008). The toxin is inhibited by cholesterol, and at high levels (greater than 50 hemolytic units), it is lytic to all cells with cholesterol in the membrane. At lower, sublytic concentrations, pneumolysin can induce apoptosis, activate the host complement, and induce proinflammatory reactions in immune cells. Pneumolysin is generally located in the bacterial cytoplasm, but does not have an N-terminal secretion signal sequence, so it is released when the pneumococcus undergoes autolysis with N-acetyl-muramoyl-1-alanine amidase (Lyt A). Hirst et al., Clin Exp Immunol. 138(2): 195-201 (2004). The toxin, a water-soluble monomer, recognizes mammalian cells via its C-terminal domain (domain 4), and assembles into circular prepores of approximately 30-50 monomers on the surface of cholesterol-rich membranes. When bound, the monomers undergo conformational changes, resulting in a PLY β-barrel pore that causes lysis of the target cell. Lawrence et al., Sci Rep. 5:14352 (2015).

Due to drug-resistant pneumococci and the complexity of vaccines needed to cover a broad spectrum of strains, it is necessary to find new vaccines with antigens that offer protection in a strain-independent manner and that can effectively abolish the virulence of S. pneumoniae.

### SUMMARY

Provided herein are highly effective bacterial vaccines that are useful for treating, prophylactically and therapeutically, bacterial infection. The bacterial vaccines are ribonucleic acid (RNA) vaccines that enable production of a bacterial protein in a host from messenger RNA (mRNA)) such that the protein can safely elicit a robust immune response. The RNA (e.g., mRNA) vaccines of the present disclosure may be used to induce a balanced immune response against bacterial infections, comprising both cellular and humoral immunity.

The RNA (e.g., mRNA) vaccines may be utilized in various settings depending on the prevalence of the infection or the degree or level of unmet medical need. The RNA (e.g. mRNA) vaccines may be utilized to treat and/or prevent a bacterial infection. The RNA (e.g., mRNA) vaccines have superior properties in that they may produce much larger antibody titers and produce responses earlier than commercially available vaccines. While not wishing to be bound by theory, it is believed that the RNA (e.g., mRNA) vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion. Additionally, the ability to produce proteins in human hosts that are not glycosylated, and thus more closely mimic naturally occurring bacterial proteins have been found to produce more robust immune responses that are effective in neutralizing the bacteria and preventing bacterial infection.

In some aspects the invention is a bacterial vaccine, comprising at least one RNA polynucleotide having an open reading frame encoding at least one bacterial antigenic polypeptide which comprises a mutated N-linked glycosylation site. In some embodiments, the mutated bacterial antigenic polypeptide comprises at least one asparagine (Asn) amino acid of a corresponding wild-type bacterial antigenic polypeptide which has been replaced with a non-Asn amino acid.

In some embodiments the mutated bacterial antigenic polypeptide has one, two, three, four, or five Asn amino acids of a corresponding wild type bacterial antigenic polypeptide which have been replaced with a non-Asn amino acid. In other embodiments the Asn amino acid has been replaced with an Ala amino acid. In some embodiments the mutated bacterial antigenic polypeptide has greater than 80%, 85%, 90%, 95%, 96%, 97%, 98%, or 99% sequence identity to a wild type bacterial antigenic polypeptide.

In some embodiments the bacterial vaccine produces a lower IgG titer than an RNA vaccine encoding a corresponding wild type antigen. In other embodiments the bacterial vaccine has enhanced neutralization activity relative to an RNA vaccine encoding a corresponding wild type antigen.

The mutated bacterial antigenic polypeptide in some embodiments is a mutated antigen of an infectious bacteria selected from the group consisting of Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Treponema, Vibrio, Walbachia, and Yersinia. In other embodiments the Streptococcus is Streptococcus pneumoniae. In yet other embodiments the mutated antigen is a pneumolysin.

In other aspects, the invention is a Streptococcal vaccine, having at least one RNA polynucleotide having an open reading frame encoding at least one Streptococcal antigenic polypeptide. In some embodiments the Streptococcus is Streptococcus pneumoniae. In yet other embodiments the mutated antigen is a pneumolysin. In some embodiments the pneumolysin has a wild type pneumolysin sequence. In other embodiments the pneumolysin has a modified pneumolysin sequence. In yet other embodiments the modified pneumolysin sequence includes a D205R mutation.

The Streptococcal vaccine in some embodiments has at least one RNA polynucleotide with a nucleic acid sequence that has at least 80%, 85%, 90%, 95%, 98%, or 99% identity to any one of SEQ ID NO: 6-8, but does not include wild-type mRNA sequence. In other embodiments the Streptococcal antigenic polypeptide has an amino acid sequence that has at least 90%, 95%, 98%, or 99% identity to an amino acid sequence identified by any one of SEQ ID NO: 10-29, but does not include wild-type protein sequence. In yet other embodiments the Streptococcal antigenic polypeptide has an amino acid sequence of any one of SEQ ID NO: 10-29. In some embodiments the at least one RNA polynucleotide has a nucleic acid sequence of any one of SEQ ID NO: 6-8.

Provided herein, in some embodiments, is a ribonucleic acid (RNA) (e.g., mRNA) vaccine, comprising at least one (e.g., at least 2, 3, 4 or 5) RNA (e.g., mRNA) polynucleotide having an open reading frame encoding at least one bacterial antigenic polypeptide, or any combination of two or more of the foregoing antigenic polypeptides. Herein, use of the term “antigenic polypeptide” encompasses immunogenic fragments of the antigenic polypeptide (an immunogenic fragment that is induces (or is capable of inducing) an immune response to a bacterial infection, unless otherwise stated.

Also provided herein, in some embodiments, is a RNA (e.g., mRNA) vaccine comprising at least one (e.g., at least 2, 3, 4 or 5) RNA polynucleotide having an open reading frame encoding at least one (e.g., at least 2, 3, 4 or 5) bacterial antigenic polypeptide or an immunogenic fragment thereof, optionally linked to a signal peptide.

Further still, provided herein, in some embodiments, is a method of inducing an immune response in a subject, the method comprising administering to the subject a vaccine comprising at least one (e.g., at least 2, 3, 4 or 5) RNA (e.g., mRNA) polynucleotide having an open reading frame encoding at least one (e.g., at least 2, 3, 4 or 5) bacterial antigenic polypeptide, or any combination of two or more of the foregoing antigenic polypeptides.

In some embodiments, at least one antigenic polypeptide is a bacterial polyprotein. In some embodiments, at least one antigenic polypeptide is pneumolysin or a pneumolysin variant (pneumolysoid).

In some embodiments, at least one bacterial antigenic polypeptide comprises an amino acid sequence identified by any one of SEQ ID NO: 10-29, 36-38 (Table 2). In some embodiments, the amino acid sequence of the bacterial antigenic polypeptide is, or is a fragment of, or is a homolog or variant having at least 80% (e.g., 85%, 90%, 95%, 98%, 99%, 80-90%, 90-95%, 90-98%, 90-99%, 80-99%) identity to, the amino acid sequence identified by any one of SEQ ID NO: 10-29, 36-38 (Table 2).

In some embodiments, the at least one bacterial antigenic polypeptide is encoded by a nucleic acid sequence from Table 1. In some embodiments, at least one bacterial antigenic polypeptide is encoded by a nucleic acid sequence identified by any one of SEQ ID NO: 2-4, 30-32, 56-61 (Table 1).

In some embodiments, at least one bacterial RNA (e.g., mRNA) polynucleotide is encoded by a nucleic acid sequence, or a fragment of a nucleotide sequence, identified by any one of SEQ ID NO: 2-4, 30-32, 56-61 (Table 1).

In some embodiments, an open reading frame of a RNA (e.g., mRNA) vaccine is codon-optimized. In some embodiments, at least one RNA polynucleotide encodes at least one antigenic polypeptide having an amino acid sequence identified by any one of SEQ ID NO: 10-29 (Table 2) and is codon optimized mRNA.

In some embodiments, a RNA (e.g., mRNA) vaccine further comprises an adjuvant.

Each of the amino acid sequences, and variants having greater than 95% identity or greater than 98% identity to each of the amino acid sequences encompassed by the accession numbers of SEQ ID NO: 9 are included within the constructs (polynucleotides/polypeptides) of the present disclosure.

In some embodiments, at least one mRNA polynucleotide is encoded by a nucleic acid having a sequence identified by any one of SEQ ID NO: 6-8, 33-35, 62-67 (Table 1) and having less than 80% identity to wild-type mRNA sequence. In some embodiments, at least one mRNA polynucleotide is encoded by a nucleic acid having a sequence identified by any one of SEQ ID NO: 6-8, 33-35, 62-67 (Table 1) and having less than 100% but greater than 75%, 85% or 95% identity to a wild-type mRNA sequence. In some embodiments, at least one mRNA polynucleotide is encoded by a nucleic acid having a sequence identified by any one of SEQ ID NO: 6-8, 33-35, 62-67 (Table 1) and having 50-80%, 60-80%, 40-80%, 30-80%, 70-80%, 75-80% or 78-80% identity to wild-type mRNA sequence. In some embodiments, at least one mRNA polynucleotide is encoded by a nucleic acid having a sequence identified by any one of SEQ ID NO: 6-8, 33-35, 62-67 (Table 1) and having 40-85%, 50-85%, 60-85%, 30-85%, 70-85%, 75-85% or 80-85% identity to wild-type mRNA sequence. In some embodiments, at least one mRNA polynucleotide is encoded by a nucleic acid having a sequence identified by any one of SEQ ID NO: 6-8, 33-35, 62-67 (Table 1) and having 40-90%, 50-90%, 60-90%, 30-90%, 70-90%, 75-90%, 80-90%, or 85-90% identity to wild-type mRNA sequence.

In some embodiments, at least one RNA polynucleotide encodes at least one antigenic polypeptide having an amino acid sequence identified by any one of SEQ ID NO: 10-29 (Table 2) and having at least 80% (e.g., 85%, 90%, 95%, 98%, 99%) identity to wild-type mRNA sequence, but does not include wild-type mRNA sequence.

In some embodiments, at least one RNA polynucleotide encodes at least one antigenic polypeptide having an amino acid sequence identified by any one of SEQ ID NO: 10-29 (Table 2) and has greater than 95%, 90%, 85%, 80% or 75% identity to wild-type mRNA sequence. In some embodiments, at least one RNA polynucleotide encodes at least one antigenic polypeptide having an amino acid sequence identified by any one of SEQ ID NO: 10-29 (Table 2) and has 30-80%, 40-80%, 50-80%, 60-80%, 70-80%, 75-80% or 78-80%, 30-85%, 40-85%, 50-85%, 60-85%, 70-85%, 75-85% or 78-85%, 30-90%, 40-90%, 50-90%, 60-90%, 70-90%, 75-90%, 80-90% or 85-90% identity to wild-type mRNA sequence.

In some embodiments, at least one RNA polynucleotide encodes at least one antigenic polypeptide having at least 90%, at least 95%, at least 96%, at least 97%, at least 98%, or at least 99% identity to an amino acid sequence identified by any one of SEQ ID NO: 10-29 (Table 2). In some embodiments, at least one RNA polynucleotide encodes at least one antigenic polypeptide having 95%-99% identity to an amino acid sequence identified by any one of SEQ ID NO: 10-29 (Table 2).

Some embodiments of the present disclosure provide a vaccine that includes at least one ribonucleic acid (RNA) (e.g., mRNA) polynucleotide having an open reading frame encoding at least one antigenic polypeptide (e.g., at least one bacterial antigenic polypeptide), at least one 5′ terminal cap and at least one chemical modification, formulated within a lipid nanoparticle.

In some embodiments, a 5′ terminal cap is 7mG(5′)ppp(5′)NlmpNp.

In some embodiments, at least one chemical modification is selected from pseudouridine, N1-methylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments, a lipid nanoparticle comprises compounds of Formula (I) and/or Formula (II), discussed below.

In some embodiments, a bacterial RNA (e.g., mRNA) vaccine is formulated in a lipid nanoparticle that comprises a compound selected from Compounds 3, 18, 20, 25, 26, 29, 30, 60, 108-112 and 122, described below.

Some embodiments of the present disclosure provide a vaccine that includes at least one RNA (e.g., mRNA) polynucleotide having an open reading frame encoding at least one antigenic polypeptide (e.g., at least one bacterial antigenic polypeptide), wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, an open reading frame of a RNA (e.g., mRNA) polynucleotide encodes at least two antigenic polypeptides (e.g., at least two bacterial antigenic polypeptides). In some embodiments, the at least two bacterial antigenic polypeptides are the same bacterial antigenic polypeptides. In other embodiments, the at least two bacterial antigenic polypeptides are different bacterial antigenic polypeptides. In some embodiments, the open reading frame encodes at least five or at least ten antigenic polypeptides. In some embodiments, the open reading frame encodes at least 100 antigenic polypeptides. In some embodiments, the open reading frame encodes 2-100 antigenic polypeptides.

In some embodiments, a vaccine comprises at least two RNA (e.g., mRNA) polynucleotides, each having an open reading frame encoding at least one antigenic polypeptide (e.g., at least one bacterial antigenic polypeptide). In some embodiments, the vaccine comprises at least five or at least ten RNA (e.g., mRNA) polynucleotides, each having an open reading frame encoding at least one antigenic polypeptide or an immunogenic fragment thereof. In some embodiments, the vaccine comprises at least 100 RNA (e.g., mRNA) polynucleotides, each having an open reading frame encoding at least one antigenic polypeptide. In some embodiments, the vaccine comprises 2-100 RNA (e.g., mRNA) polynucleotides, each having an open reading frame encoding at least one antigenic polypeptide.

In some embodiments, at least one antigenic polypeptide (e.g., at least one bacterial antigenic polypeptide) is fused to a signal peptide. In some embodiments, the signal peptide is selected from: a HulgGk signal peptide (METPAQLLFLLLLWLPDTTG; SEQ ID NO: 39); IgE heavy chain epsilon-1 signal peptide (MDWTWILFLVAAATRVHS; SEQ ID NO: 40); Japanese encephalitis PRM signal sequence (MLGSNSGQRVVFTILLLLVAPAYS; SEQ ID NO: 41), VSVg protein signal sequence (MKCLLYLAFLFIGVNCA; SEQ ID NO: 42) and Japanese encephalitis JEV signal sequence (MWLVSLAIVTACAGA; SEQ ID NO: 43).

In some embodiments, the signal peptide is fused to the N-terminus of at least one antigenic polypeptide. In some embodiments, a signal peptide is fused to the C-terminus of at least one antigenic polypeptide.

Also provided herein is a RNA (e.g., mRNA) vaccine of any embodiments of the invention formulated in a nanoparticle (e.g., a lipid nanoparticle or cationic lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 5-25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In some embodiments, a lipid nanoparticle comprises compounds of Formula (I) and/or Formula (II), as discussed below.

In some embodiments, a lipid nanoparticle comprises Compounds 3, 18, 20, 25, 26, 29, 30, 60, 108-112, or 122, as discussed below.

In some embodiments, the nanoparticle has a polydispersity value of less than 0.4 (e.g., less than 0.3, 0.2 or 0.1).

In some embodiments, the nanoparticle has a net neutral charge at a neutral pH value.

In some embodiments, the bacterial vaccine is multivalent.

Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is a bacterial vaccine. In other embodiments, the RNA (e.g., mRNA) vaccine is a streptococcal vaccine.

In some embodiments, an antigen-specific immune response comprises a T cell response or a B cell response.

In some embodiments, a method of producing an antigen-specific immune response comprises administering to a subject a single dose (no booster dose) of a RNA (e.g., mRNA) vaccine of the present disclosure. In some embodiments, a method further comprises administering to the subject a second (booster) dose of a RNA (e.g., mRNA) vaccine. Additional doses of a RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a bacteria can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an RNA (e.g., mRNA) vaccine is administered to a subject by intradermal or intramuscular injection.

Some embodiments of the present disclosure provide methods of inducing an antigen specific immune response in a subject, including administering to a subject a RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to a bacterial antigenic polypeptide) following administration to the subject of any of the RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated bacterial vaccine, or wherein the control is an anti-antigenic polypeptide antibody titer produced in a subject who has been administered a recombinant or purified bacterial protein vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant bacterial protein vaccine, wherein the anti-antigenic polypeptide antibody titer produced in the subject is equivalent to an anti-antigenic polypeptide antibody titer produced in a control subject administered the standard of care dose of a recombinant bacterial protein vaccine, a purified bacterial protein vaccine, a live attenuated bacterial vaccine, or an inactivated bacterial vaccine. In some embodiments, the effective amount is a dose equivalent to 2-1000-fold reduction in the standard of care dose of a recombinant bacterial protein vaccine, wherein the anti-antigenic polypeptide antibody titer produced in the subject is equivalent to an anti-antigenic polypeptide antibody titer produced in a control subject administered the standard of care dose of a recombinant bacterial protein vaccine, a purified bacterial protein vaccine, a live attenuated bacterial vaccine, or an inactivated bacterial vaccine.

In some embodiments, the RNA (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a total dose of 1-100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg, 5 μg-100 μg, 10 μg-100 μg, 15 μg-100 μg, 20 μg-100 μg, 25 μg-100 μg, 25 μg-500 μg, 50 μg-100 μg, 50 μg-500 μg, 50 μg-1000 μg, 100 μg-500 μg, 100 μg-1000 μg, 250 μg-500 μg, 250 μg-1000 μg, or 500 μg-1000 μg which may be administered to the subject a total of one or two times or more.

In some embodiments, the efficacy (or effectiveness) of a RNA (e.g., mRNA) vaccine is greater than 60%. In some embodiments, the RNA (e.g., mRNA) polynucleotide of the vaccine comprises at least one bacterial antigenic polypeptide.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against the bacteria for up to 2 years. In some embodiments, the vaccine immunizes the subject against the bacteria for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is pregnant (e.g., in the first, second or third trimester) when administered an RNA (e.g., mRNA) vaccine. Bacterial infections may cause infections of the respiratory tract, mainly in infants and young children. Pneumonia is the single largest infectious disease cause of death in children worldwide, killing 920,163 children under the age of 5 in 2015 and accounting for approximately 16% of the deaths of children under the age of 5. Thus, the present disclosure provides RNA (e.g., mRNA) vaccines for maternal immunization to improve mother-to-child transmission of protection against the bacteria.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is has a chronic pulmonary disease (e.g., chronic obstructive pulmonary disease (COPD) or asthma). Two forms of COPD include chronic bronchitis, which involves a long-term cough with mucus, and emphysema, which involves damage to the lungs over time. Thus, a subject administered a RNA (e.g., mRNA) vaccine may have chronic bronchitis or emphysema.

In some embodiments, the subject has been exposed to one or more bacteria.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first bacterial antigenic polypeptide, wherein the RNA polynucleotide does not include a stabilization element, and wherein an adjuvant is not coformulated or co-administered with the vaccine.

In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having decreased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer is a neutralizing antibody titer.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no chemical modification, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no chemical modification, the open reading frame encoding a first antigenic polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 ug and 400 ug of one or more RNA polynucleotides having an open reading frame comprising at least one chemical modification or optionally no chemical modification, the open reading frame encoding a first antigenic polypeptide, and a pharmaceutically acceptable carrier or excipient, formulated for delivery to a human subject. In some embodiments, the vaccine further comprises a cationic lipid nanoparticle.

The data presented in the Examples demonstrate significant enhanced immune responses using the formulations of the invention. The data demonstrated the effectiveness of mutating N-glycosylation sites in the RNA vaccines of the invention. Surprisingly, it was discovered herein that N-glycosylation mutated mRNA vaccines has a positive effect on bacterial neutralization, even when lower levels of IgG are observed.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of various embodiments of the disclosure.

FIGS. 1A-1B show the results of serum IgG anti-pneumolysin assays 21 days after the first immunization (FIG. 1A) and 41 days after the first immunization (FIG. 1B).

FIG. 2 shows a hemolytic unit (HU) determination curve. The pneumolysin concentration for 50% hemolysis was found to be 5 ng/mL.

FIG. 3 shows the results of a serum neutralization assay using 4 HU. The serum used was from the 41 day, 10 μg dose groups.

FIG. 4 shows shows the expression of mRNAs encoding previously characterized pneumolysin toxoid variants in HEK293F cells. The pneumolysin-predicted molecular weight was 53 kDa, and E. coli produced runs approximately 62 kDa. Detection was with rabbit anti-His pAb (abcam ab9108) (light gray) and mouse anti-beta actin mAb (dark gray). The lack of expression in the L460D NGM group was likely due to a technical error, as all variants were well—expressed and the construct immunogenic in vivo. L, lysate, S, supernatant, cS, concentrated supernatant, WT, wild type, and NGM, N-linked glycosylation mutant.

FIGS. 5A-5B show the expression of mRNA novel pneumolysin toxoid variants in HEK293F cells. Detection was with rabbit anti-His pAb (abcam ab9108) (green) and mouse anti-beta actin mAb (red). L, lysate, S, supernatant, cS, concentrated supernatant, WT, wild type, and NGM, N-linked glycosylation mutant.

FIG. 6 shows the results of a serum neutralization assay using 4 HU. The serum used was from the 41 day, 2 μg and 10 μg dose groups.

FIG. 7 shows the expression of mRNA PspA variants from the TIGR4 and Rx1 strains in HEK293 cells. Detection was with rabbit anti-His (A1647) (light gray) and mouse anti-actin mAb (Cy3) (dark gray).

FIG. 8 shows the expression of mRNA CbpA variants from the TIGR4 strain in HEK293 cells. Instead of using untransfected cells as a control, positive PspA was used. Detection was with rabbit anti-His (A1647) (light gray) and mouse anti-actin mAb (Cy3) (dark gray) (left blot) and with rabbit anti-His (A1647) (right blot).

FIG. 9 shows the expression of mRNA PhtD, PiaA, and PiuA variants in HEK293 cells. Detection was with rabbit anti-His (A1647) (light gray) and mouse anti-actin mAb (Cy3) (dark gray).

FIG. 10 shows the expression of mRNA PcsB and SktP in HEK293 cells. Detection was with rabbit anti-His (A1647) (light gray) and mouse anti-actin mAb (Cy3) (dark gray).

FIG. 11 shows the expression of mRNA PsaA and PcpA in HEK293 cells. Detection was with rabbit anti-His (A1647) (light gray) and mouse anti-actin mAb (Cy3) (dark gray).

FIG. 12 shows the expression of mRNA StkP and PhtE in HEK293 cells. Detection was with rabbit anti-His (A1647) (light gray) and mouse anti-actin mAb (Cy3) (dark gray).

FIG. 13 shows the results of a serum IgG anti-PspA assay on day 42 of an in vivo immunogenicity study testing PspA vaccine constructs.

FIG. 14 shows the results of a serum IgG anti-CbpA assay on days 20 and 42 of an in vivo immunogenicity study testing CbpA vaccine constructs.

FIG. 15 shows the results of a serum IgG anti-PiaA assay on days 21 and 41 of an in vivo immunogenicity study testing PiaA vaccine constructs.

FIG. 16 shows the results of a serum IgG anti-PiuA assay on days 21 and 41 of an in vivo immunogenicity study testing PiuA vaccine constructs.

FIG. 17 shows the results of a serum IgG anti-PhtD assay on days 22 and 36 of an in vivo immunogenicity study testing PhtD vaccine constructs.

FIG. 18 shows the results of six cytokine assays used to determine the cytokine response in splenic samples.

### DETAILED DESCRIPTION

The bacterial RNA vaccines described herein are superior to current vaccines in several ways. For example, the lipid nanoparticle (LNP) delivery system used herein increases the efficacy of RNA vaccines in comparison to other formulations, including a protamine-based approach described in the literature. The use of this LNP delivery system enables the effective delivery of chemically-modified RNA vaccines or unmodified RNA vaccines, without requiring additional adjuvant to produce a therapeutic result (e.g., production neutralizing antibody titer and/or a T cell response). In some embodiments, the bacterial RNA vaccines disclosed herein are superior to conventional vaccines by a factor of at least 10 fold, 20, fold, 40, fold, 50 fold, 100 fold, 500 fold, or 1,000 fold when administered intramuscularly (IM) or intradermally (ID). These results can be achieved even when significantly lower doses of the RNA (e.g., mRNA) are administered in comparison with RNA doses used in other classes of lipid based formulations.

The LNP used in the studies described herein has been used previously to deliver siRNA in various animal models as well as in humans. In view of the observations made in association with the siRNA delivery of LNP formulations, the fact that LNP is useful in vaccines is quite surprising, particularly when immunity to an antigen has been hard to generate, as in the case of bacterial infections. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the present disclosure are demonstrated herein to generate enhanced IgG levels, sufficient for prophylactic and therapeutic methods rather than transient IgM responses.

The present disclosure provides, in some embodiments, vaccines that comprise RNA (e.g., mRNA) polynucleotides encoding a bacterial antigenic polypeptide. Also provided herein are methods of administering the RNA (e.g., mRNA) vaccines, methods of producing the RNA (e.g., mRNA) vaccines, compositions (e.g., pharmaceutical compositions) comprising the RNA (e.g., mRNA) vaccines, and nucleic acids (e.g., DNA) encoding the RNA (e.g., mRNA) vaccines. In some embodiments, a RNA (e.g., mRNA) vaccine comprises an adjuvant, such as a flagellin adjuvant, as provided herein.

The RNA (e.g., mRNA) bacterial vaccines, in some embodiments, may be used to induce a balanced immune response, comprising both cellular and humoral immunity, without many of the risks associated with DNA vaccination.

Although attempts have been made to produce functional recombinant bacterial vaccines, the therapeutic efficacy of these vaccines have not yet been fully established. Quite surprisingly, the inventors have discovered, according to aspects of the invention that a class of formulations for delivering mRNA vaccines in vivo that results in significantly enhanced, immune responses including enhanced neutralization capability. The formulations of the invention have demonstrated significant unexpected immune responses sufficient to establish the efficacy of functional bacterial mRNA vaccines as prophylactic and therapeutic agents.

In certain aspects, the invention provides a polypeptide comprising mutated N-linked glycosylation sites. N-linked glycans of bacterial proteins play important roles in modulating the immune response. Glycans can be important for maintaining the appropriate antigenic conformations, shielding potential neutralization epitopes, and may alter the proteolytic susceptibility of proteins. Some bacteria have putative N-linked glycosylation sites. Deletion or modification of an N-linked glycosylation site may enhance the immune response. Thus, the present disclosure provides, in some embodiments, RNA (e.g., mRNA) vaccines comprising nucleic acids (e.g., mRNA) encoding antigenic polypeptides that comprise a deletion or modification at one or more N-linked glycosylation sites. N-linked glycosylation, the attachment of a glycan (sugar molecule oligosaccharide) to the amide nitrogen of an asparagine residue, occurs frequently in eukaryotes but rarely in bacteria. Imperiali et al., Curr Opin in Chem Biol. 3(6):643-649 (1999). Without wishing to be bound by any theory, it is thought that expressing the bacterial protein in a mammalian cell may lead to glycosylated antigens having altered or ineffective immunogenicity profiles. Thus, in some embodiments, the pneumolysin polypeptide comprises one or more n-linked glycosylation site(s).

Pneumolysin (PLY, AJS15225.1; M17717.1), a putative major virulence factor of Streptococcus pneumoniae, is a 53 kDa pore-forming toxin consisting of 471 amino acids. Marriott et al., Curr Mol Med. 8(6):497-509 (2008). The toxin is inhibited by cholesterol, and at high levels (greater than 50 hemolytic units), it is lytic to all cells with cholesterol in the membrane. At lower, sublytic concentrations, pneumolysin can induce apoptosis, activate the host complement, and induce proinflammatory reactions in immune cells. Pneumolysin is generally located in the bacterial cytoplasm, but does not have an N-terminal secretion signal sequence, so it is released when the pneumococcus undergoes autolysis with N-acetyl-muramoyl-1-alanine amidase (Lyt A). Hirst et al., Clin Exp Immunol. 138(2): 195-201 (2004). The toxin, a water-soluble monomer, recognizes mammalian cells via its C-terminal domain (domain 4), and assembles into circular prepores of approximately 30-50 monomers on the surface of cholesterol-rich membranes. When bound, the monomers undergo conformational changes, resulting in a PLY β-barrel pore that causes lysis of the target cell. Lawrence et al., Sci Rep. 5:14352 (2015).

The invention in aspects is a composition of an RNA polynucleotide comprising an open reading frame (ORF) encoding pneumolysin (PLY) polypeptide which may be formulated in a cationic lipid nanoparticle. The PLY may be a wild type PLY or a variant polypeptide. The compositions of the invention have several advantages over prior art methods for treating pneumococcal infections, including prior art PLY formulations such as protein or nucleic acid PLY formulations.

Bacterial vaccines, as provided herein, comprise at least one (one or more) ribonucleic acid (RNA) (e.g., mRNA) polynucleotide having an open reading frame encoding at least one mutated antigenic polypeptide selected from Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Treponema, Vibrio, Walbachia, and Yersinia antigenic polypeptides and containing at least one N-linked glycosylation mutation. The term “nucleic acid” includes any compound and/or substance that comprises a polymer of nucleotides (nucleotide monomer). These polymers are referred to as polynucleotides. Thus, the terms “nucleic acid” and “polynucleotide” are used interchangeably.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

In some embodiments, a RNA polynucleotide of an RNA (e.g., mRNA) vaccine encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 antigenic polypeptides. In some embodiments, a RNA (e.g., mRNA) polynucleotide of a bacterial vaccine encodes at least 10, 20, 30, 40, 50, 60, 70, 80, 90 or 100 antigenic polypeptides. In some embodiments, a RNA (e.g., mRNA) polynucleotide of a bacterial vaccine encodes at least 100 or at least 200 antigenic polypeptides. In some embodiments, a RNA polynucleotide of bacterial vaccine encodes 1-10, 5-15, 10-20, 15-25, 20-30, 25-35, 30-40, 35-45, 40-50, 1-50, 1-100, 2-50 or 2-100 antigenic polypeptides.

### Codon Optimization

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an XXX antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, an antigenic polypeptide is longer than 25 amino acids and shorter than 50 amino acids. Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, such as antibodies or insulin, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments “variant mimics” are provided. A “variant mimic” contains at least one amino acid that would mimic an activated sequence. For example, glutamate may serve as a mimic for phosphoro-threonine and/or phosphoro-serine. Alternatively, variant mimics may result in deactivation or in an inactivated product containing the mimic. For example, phenylalanine may act as an inactivating substitution for tyrosine, or alanine may act as an inactivating substitution for serine.

“Orthologs” refers to genes in different species that evolved from a common ancestral gene by speciation. Normally, orthologs retain the same function in the course of evolution. Identification of orthologs is important for reliable prediction of gene function in newly sequenced genomes.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Multiprotein and Multicomponent Vaccines

The present disclosure encompasses bacterial vaccines comprising multiple RNA (e.g., mRNA) polynucleotides, each encoding a single antigenic polypeptide, as well as bacterial vaccines comprising a single RNA polynucleotide encoding more than one antigenic polypeptide (e.g., as a fusion polypeptide). Thus, a vaccine composition comprising a RNA (e.g., mRNA) polynucleotide having an open reading frame encoding a first antigenic polypeptide and a RNA (e.g., mRNA) polynucleotide having an open reading frame encoding a second antigenic polypeptide encompasses (a) vaccines that comprise a first RNA polynucleotide encoding a first antigenic polypeptide and a second RNA polynucleotide encoding a second antigenic polypeptide, and (b) vaccines that comprise a single RNA polynucleotide encoding a first and second antigenic polypeptide (e.g., as a fusion polypeptide). RNA (e.g., mRNA) vaccines of the present disclosure, in some embodiments, comprise 2-10 (e.g., 2, 3, 4, 5, 6, 7, 8, 9 or 10), or more, RNA polynucleotides having an open reading frame, each of which encodes a different antigenic polypeptide (or a single RNA polynucleotide encoding 2-10, or more, different antigenic polypeptides). The antigenic polypeptides may be selected from bacterial antigenic polypeptides.

In some embodiments, a multicomponent vaccine comprises at least one RNA (e.g., mRNA) polynucleotide encoding at least one antigenic polypeptide fused to a signal peptide (e.g., any one of SEQ ID NO: 39-43). The signal peptide may be fused at the N-terminus or the C-terminus of an antigenic polypeptide.

### Signal Peptides

In some embodiments, antigenic polypeptides encoded by bacterial RNA (e.g., mRNA) polynucleotides comprise a signal peptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Bacterial vaccines of the present disclosure may comprise, for example, RNA (e.g., mRNA) polynucleotides encoding an artificial signal peptide, wherein the signal peptide coding sequence is operably linked to and is in frame with the coding sequence of the antigenic polypeptide. Thus, bacterial vaccines of the present disclosure, in some embodiments, produce an antigenic polypeptide comprising an antigenic polypeptide fused to a signal peptide. In some embodiments, a signal peptide is fused to the N-terminus of the antigenic polypeptide. In some embodiments, a signal peptide is fused to the C-terminus of the antigenic polypeptide.

In some embodiments, the signal peptide fused to the antigenic polypeptide is an artificial signal peptide. In some embodiments, an artificial signal peptide fused to the antigenic polypeptide encoded by the RNA (e.g., mRNA) vaccine is obtained from an immunoglobulin protein, e.g., an IgE signal peptide or an IgG signal peptide. In some embodiments, a signal peptide fused to the antigenic polypeptide encoded by a RNA (e.g., mRNA) vaccine is an Ig heavy chain epsilon-1 signal peptide (IgE HC SP) having the sequence of: MDWTWILFLVAAATRVHS (SEQ ID NO: 40). In some embodiments, a signal peptide fused to the antigenic polypeptide encoded by the (e.g., mRNA) RNA (e.g., mRNA) vaccine is an IgGk chain V-III region HAH signal peptide (IgGk SP) having the sequence of METPAQLLFLLLLWLPDTTG (SEQ ID NO: 39). In some embodiments, the signal peptide is selected from: Japanese encephalitis PRM signal sequence (MLGSNSGQRVVFTILLLLVAPAYS; SEQ ID NO: 41), VSVg protein signal sequence (MKCLLYLAFLFIGVNCA; SEQ ID NO: 42) and Japanese encephalitis JEV signal sequence (MWLVSLAIVTACAGA; SEQ ID NO: 43).

In some embodiments, the antigenic polypeptide encoded by a RNA (e.g., mRNA) vaccine comprises an amino acid sequence identified by any one of SEQ ID NO: 10-29 (Table 2) fused to a signal peptide identified by any one of SEQ ID NO: 39-43. The examples disclosed herein are not meant to be limiting and any signal peptide that is known in the art to facilitate targeting of a protein to ER for processing and/or targeting of a protein to the cell membrane may be used in accordance with the present disclosure.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

A signal peptide is typically cleaved from the nascent polypeptide at the cleavage junction during ER processing. The mature antigenic polypeptide produce by a bacterial RNA (e.g., mRNA) vaccine of the present disclosure typically does not comprise a signal peptide.

### Fusion Proteins

In some embodiments, a bacterial RNA vaccine of the present disclosure includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the antigen. Antigenic fusion proteins, in some embodiments, retain the functional property from each original protein.

### Scaffold Moieties

The RNA (e.g., mRNA) vaccines as provided herein, in some embodiments, encode fusion proteins which comprise bacterial antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments a bacterial antigen is fused to HBsAG or HBcAG to facilitate self-assembly of nanoparticles displaying the bacterial antigen.

In another embodiment, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 Å peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain. Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Chemically Unmodified Nucleotides

In some embodiments, at least one RNA (e.g., mRNA) of a bacterial vaccine of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

Bacterial vaccines of the present disclosure, in some embodiments, comprise at least RNA (e.g. mRNA) polynucleotide having an open reading frame encoding at least one antigenic polypeptide, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the vaccines of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladeno sine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adeno sine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m⁵C). In some embodiments, polynucleotides (e.g., RNA polynucleotides, such as mRNA polynucleotides) comprise 1-methyl-pseudouridine (m¹ψ). In some embodiments, polynucleotides (e.g., RNA polynucleotides, such as mRNA polynucleotides) comprise 1-methyl-pseudouridine (m¹ψ) and 5-methyl-cytidine (m⁵C). In some embodiments, polynucleotides (e.g., RNA polynucleotides, such as mRNA polynucleotides) comprise 2-thiouridine (s²U). In some embodiments, polynucleotides (e.g., RNA polynucleotides, such as mRNA polynucleotides) comprise 2-thiouridine and 5-methyl-cytidine (m⁵C). In some embodiments, polynucleotides (e.g., RNA polynucleotides, such as mRNA polynucleotides) comprise methoxy-uridine (mo⁵U). In some embodiments, polynucleotides (e.g., RNA polynucleotides, such as mRNA polynucleotides) comprise 5-methoxy-uridine (mo⁵U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m⁶A). In some embodiments, polynucleotides (e.g., RNA polynucleotides, such as mRNA polynucleotides) comprise N6-methyl-adenosine (m⁶A) and 5-methyl-cytidine (m⁵C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. In some embodiments, a modified nucleobase is a modified cytosine. nucleosides having a modified uridine include 5-cyano uridine and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). n some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA (e.g., mRNA) vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A) 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guano sine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, 2′-F-ara-guanosine, and 2′-F-guanosine.

### Untranslated Regions (UTRs)

The nucleic acids of the present disclosure may comprise one or more regions or parts which act or function as an untranslated region. Where nucleic acids are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US2014/0206753, WO2013/185069), the sequence GGGAUCCUACC (SEQ ID NO: 68) (WO2014/144196) may also be used. In another embodiment, 5′ UTR of a TOP gene is a 5′ UTR of a TOP gene lacking the 5′ TOP motif (the oligopyrimidine tract) (e.g., WO2015/101414, WO2015/101415, WO2015/062738, WO2015/024667, WO2015/024668); 5′ UTR element derived from ribosomal protein Large 32 (L32) gene (WO2015/101414, WO2015/101415, WO2015/062738), 5′ UTR element derived from the 5′UTR of an hydroxysteroid (1743) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

In some embodiments, a 5′ UTR of the present disclosure comprises one of SEQ ID NO: 54 or SEQ ID NO: 69.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

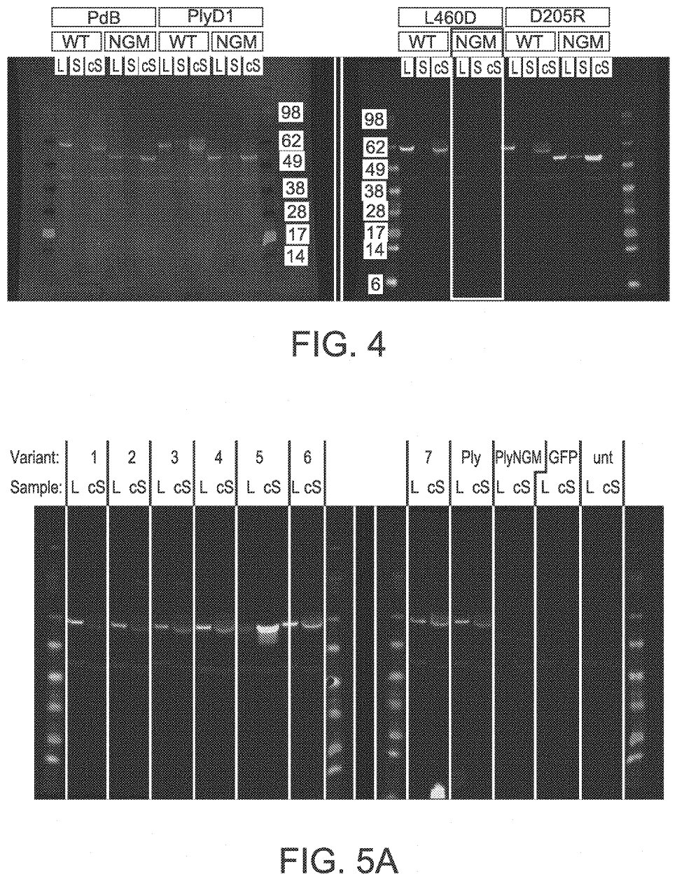

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection. 3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US2011/0086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US2014/0206753, WO2014/152774), rabbit β globin and hepatitis B virus (HBV), α-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps9 3′UTR (WO2015/101414), FIG. 4 (WO2015/101415), and human albumin 7 (WO2015/101415).

In some embodiments, a 3′ UTR of the present disclosure comprises SEQ ID NO: 55.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art.
In Vitro Transcription of RNA (e.g., mRNA)

cDNA encoding the polynucleotides described herein may be transcribed using an in vitro transcription (IVT) system. In vitro transcription of RNA is known in the art and is described in International Publication WO2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to RNA encoding an antigenic polypeptide, e.g. mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5 ‘ to and operably linked to the gene of interest.

In some embodiments, an in vitro transcription template encodes a 5’ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-Phase Chemical Synthesis.

Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis.

The synthesis of nucleic acids of the present disclosure by the sequential addition of monomer building blocks may be carried out in a liquid phase.

Combination of Synthetic Methods.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Flagellin Adjuvants

Flagellin is an approximately 500 amino acid monomeric protein that polymerizes to form the flagella associated with bacterial motion. Flagellin is expressed by a variety of flagellated bacteria (Salmonella typhimurium for example) as well as non-flagellated bacteria (such as Escherichia coli). Sensing of flagellin by cells of the innate immune system (dendritic cells, macrophages, etc.) is mediated by the Toll-like receptor 5 (TLR5) as well as by Nod-like receptors (NLRs) Ipaf and Naip5. TLRs and NLRs have been identified as playing a role in the activation of innate immune response and adaptive immune response. As such, flagellin provides an adjuvant effect in a vaccine.

The nucleotide and amino acid sequences encoding known flagellin polypeptides are publicly available in the NCBI GenBank database. The flagellin sequences from S. Typhimurium, H. Pylori, V. Cholera, S. marcesens, S. flexneri, T. Pallidum, L. pneumophila, B. burgdorferei, C. difficile, R. meliloti, A. tumefaciens, R. lupini, B. clarridgeiae, P. Mirabilis, B. subtilus, L. monocytogenes, P. aeruginosa, and E. coli, among others are known.

A flagellin polypeptide, as used herein, refers to a full length flagellin protein, immunogenic fragments thereof, and peptides having at least 50% sequence identify to a flagellin protein or immunogenic fragments thereof. Exemplary flagellin proteins include flagellin from Salmonella typhi (UniPro Entry number: Q56086), Salmonella typhimurium (A0A0C9DG09), Salmonella enteritidis (A0A0C9BAB7), and Salmonella choleraesuis (Q6V2X8), and SEQ ID NO: 47-49. In some embodiments, the flagellin polypeptide has at least 60%, 70%, 75%, 80%, 90%, 95%, 97%, 98%, or 99% sequence identify to a flagellin protein or immunogenic fragments thereof.

In some embodiments, the flagellin polypeptide is an immunogenic fragment. An immunogenic fragment is a portion of a flagellin protein that provokes an immune response. In some embodiments, the immune response is a TLR5 immune response. An example of an immunogenic fragment is a flagellin protein in which all or a portion of a hinge region has been deleted or replaced with other amino acids. For example, an antigenic polypeptide may be inserted in the hinge region. Hinge regions are the hypervariable regions of a flagellin. Hinge regions of a flagellin are also referred to as “D3 domain or region, “propeller domain or region,” “hypervariable domain or region” and “variable domain or region.” “At least a portion of a hinge region,” as used herein, refers to any part of the hinge region of the flagellin, or the entirety of the hinge region. In other embodiments an immunogenic fragment of flagellin is a 20, 25, 30, 35, or 40 amino acid C-terminal fragment of flagellin.

The flagellin monomer is formed by domains D0 through D3. D0 and D1, which form the stem, are composed of tandem long alpha helices and are highly conserved among different bacteria. The D1 domain includes several stretches of amino acids that are useful for TLR5 activation. The entire D1 domain or one or more of the active regions within the domain are immunogenic fragments of flagellin. Examples of immunogenic regions within the D1 domain include residues 88-114 and residues 411-431 (in Salmonella typhimurium FliC flagellin). Within the 13 amino acids in the 88-100 region, at least 6 substitutions are permitted between Salmonella flagellin and other flagellins that still preserve TLR5 activation. Thus, immunogenic fragments of flagellin include flagellin like sequences that activate TLR5 and contain a 13 amino acid motif that is 53% or more identical to the Salmonella sequence in 88-100 of FliC (LQRVRELAVQSAN; SEQ ID NO: 53).

In some embodiments, the RNA (e.g., mRNA) vaccine includes an RNA that encodes a fusion protein of flagellin and one or more antigenic polypeptides. A “fusion protein” as used herein, refers to a linking of two components of the construct. In some embodiments, a carboxy-terminus of the antigenic polypeptide is fused or linked to an amino terminus of the flagellin polypeptide. In other embodiments, an amino-terminus of the antigenic polypeptide is fused or linked to a carboxy-terminus of the flagellin polypeptide. The fusion protein may include, for example, one, two, three, four, five, six or more flagellin polypeptides linked to one, two, three, four, five, six or more antigenic polypeptides. When two or more flagellin polypeptides and/or two or more antigenic polypeptides are linked such a construct may be referred to as a “multimer.”

Each of the components of a fusion protein may be directly linked to one another or they may be connected through a linker. For instance, the linker may be an amino acid linker. The amino acid linker encoded for by the RNA (e.g., mRNA) vaccine to link the components of the fusion protein may include, for instance, at least one member selected from the group consisting of a lysine residue, a glutamic acid residue, a serine residue and an arginine residue. In some embodiments the linker is 1-30, 1-25, 1-25, 5-10, 5, 15, or 5-20 amino acids in length.

In other embodiments the RNA (e.g., mRNA) vaccine includes at least two separate RNA polynucleotides, one encoding one or more antigenic polypeptides and the other encoding the flagellin polypeptide. The at least two RNA polynucleotides may be co-formulated in a carrier such as a lipid nanoparticle.

Broad Spectrum RNA (e.g., mRNA) Vaccines

There may be situations where persons are at risk for infection with more than one strain of Streptococcus, Staphylococcus, or other bacteria. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one strain of a bacterial infection, a combination vaccine can be administered that includes RNA (e.g., mRNA) encoding at least one antigenic polypeptide protein (or antigenic portion thereof) of a first bacterium and further includes RNA encoding at least one antigenic polypeptide protein (or antigenic portion thereof) of a second bacterium. Thus, the vaccines of the present disclosure may be combination vaccines that target one or more antigens of the same strain/species, or one or more antigens of different strains/species, e.g., antigens which induce immunity to organisms which are found in the same geographic areas where the risk of certain bacterial infections is high or organisms to which an individual is likely to be exposed to when exposed to a certain bacterium. RNA (e.g., mRNA) can be co-formulated, for example, in a single lipid nanoparticle (LNP) or can be formulated in separate LNPs for co-administration.

### Methods of Treatment

Provided herein are compositions (e.g., pharmaceutical compositions), methods, kits and reagents for prevention and/or treatment of bacterial infections in humans and other mammals. Bacterial RNA (e.g. mRNA) vaccines can be used as therapeutic or prophylactic agents, alone or in combination with other vaccine(s). They may be used in medicine to prevent and/or treat bacterial infections. In exemplary aspects, the RNA (e.g., mRNA) vaccines of the present disclosure are used to provide prophylactic protection from bacterial infections. Prophylactic protection from bacterial infections can be achieved following administration of a RNA (e.g., mRNA) vaccine of the present disclosure. Bacterial RNA (e.g., mRNA) vaccines of the present disclosure may be used to treat or prevent “co-infections” containing two or more bacterial infections. Vaccines can be administered once, twice, three times, four times or more, but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against one or more bacterial infections is provided in aspects of the present disclosure. The method involves administering to the subject a bacterial RNA (e.g., mRNA) vaccine comprising at least one RNA (e.g., mRNA) polynucleotide having an open reading frame encoding at least one bacterial antigenic polypeptide thereof, thereby inducing in the subject an immune response specific to the bacterial antigenic polypeptide or an immunogenic fragment thereof, wherein anti-antigenic polypeptide antibody titer in the subject is increased following vaccination relative to anti-antigenic polypeptide antibody titer in a subject vaccinated with a prophylactically effective dose of a traditional vaccine against bacterial infections. An “anti-antigenic polypeptide antibody” is a serum antibody the binds specifically to the antigenic polypeptide.

In some embodiments, a RNA (e.g., mRNA) vaccine capable of eliciting an immune response is administered intramuscularly via a composition including a compound according to Formula (I), (IA), (II), (IIa), (IIb), (IIc), (IId) or (IIe) (e.g., Compound 3, 18, 20, 25, 26, 29, 30, 60, 108-112, or 122).

A prophylactically effective dose is a therapeutically effective dose that prevents infection with the bacteria at a clinically acceptable level. In some embodiments the therapeutically effective dose is a dose listed in a package insert for the vaccine. A traditional vaccine, as used herein, refers to a vaccine other than the RNA (e.g., mRNA) vaccines of the present disclosure. For instance, a traditional vaccine includes but is not limited to live/attenuated microorganism vaccines, killed/inactivated microorganism vaccines, subunit vaccines, protein antigen vaccines, DNA vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against one or more bacterial infections.

In some embodiments the anti-antigenic polypeptide antibody titer in the subject is increased 1 log, 2 log, 3 log, 5 log or 10 log following vaccination relative to anti-antigenic polypeptide antibody titer in a subject vaccinated with a prophylactically effective dose of a traditional vaccine against one or more bacterial infections.

A method of eliciting an immune response in a subject against one or more bacterial infections is provided in other aspects of the disclosure. The method involves administering to the subject a bacterial RNA (e.g., mRNA) vaccine comprising at least one RNA (e.g., mRNA) polynucleotide having an open reading frame encoding at least one bacterial antigenic polypeptide or an immunogenic fragment thereof, thereby inducing in the subject an immune response specific to a bacterial antigenic polypeptide or an immunogenic fragment thereof, wherein the immune response in the subject is equivalent to an immune response in a subject vaccinated with a traditional vaccine against the bacterial infection at 2 times to 100 times the dosage level relative to the RNA (e.g., mRNA) vaccine.

In some embodiments, the immune response in the subject is equivalent to an immune response in a subject vaccinated with a traditional vaccine at 2, 3, 4, 5, 10, 50, 100 times the dosage level relative to the bacterial RNA (e.g., mRNA) vaccine.

In some embodiments the immune response in the subject is equivalent to an immune response in a subject vaccinated with a traditional vaccine at 10-100 times, or 100-1000 times, the dosage level relative to the bacterial RNA (e.g., mRNA) vaccine.

In some embodiments the immune response is assessed by determining antibody titer in the subject.

Some aspects of the present disclosure provide a method of eliciting an immune response in a subject against a In some embodiments the immune response in the subject is equivalent to an immune response in a subject vaccinated with a traditional vaccine at 2, 3, 4, 5, 10, 50, 100 times the dosage level relative to the bacterial RNA (e.g., mRNA) vaccine by administering to the subject a bacterial RNA (e.g., mRNA) vaccine comprising at least one RNA (e.g., mRNA) polynucleotide having an open reading frame encoding at least one bacterial antigenic polypeptide, thereby inducing in the subject an immune response specific to the antigenic polypeptide or an immunogenic fragment thereof, wherein the immune response in the subject is induced 2 days to 10 weeks earlier relative to an immune response induced in a subject vaccinated with a prophylactically effective dose of a traditional vaccine against the bacterial infection(s). In some embodiments, the immune response in the subject is induced in a subject vaccinated with a prophylactically effective dose of a traditional vaccine at 2 times to 100 times the dosage level relative to the RNA (e.g., mRNA) vaccine.

In some embodiments, the immune response in the subject is induced 2 days earlier, or 3 days earlier, relative to an immune response induced in a subject vaccinated with a prophylactically effective dose of a traditional vaccine.

In some embodiments the immune response in the subject is induced 1 week, 2 weeks, 3 weeks, 5 weeks, or 10 weeks earlier relative to an immune response induced in a subject vaccinated with a prophylactically effective dose of a traditional vaccine.

Also provided herein is a method of eliciting an immune response in a subject against a bacterial infection by administering to the subject a bacterial RNA (e.g., mRNA) vaccine having an open reading frame encoding a first antigenic polypeptide, wherein the RNA polynucleotide does not include a stabilization element, and wherein an adjuvant is not co-formulated or co-administered with the vaccine.

### Therapeutic and Prophylactic Compositions

Provided herein are compositions (e.g., pharmaceutical compositions), methods, kits and reagents for prevention, treatment or diagnosis of bacterial infections in humans and other mammals, for example. Bacterial RNA (e.g. mRNA) vaccines can be used as therapeutic or prophylactic agents. They may be used in medicine to prevent and/or treat infectious disease. In some embodiments, the bacterial RNA (e.g., mRNA) vaccines of the present disclosure are used for the priming of immune effector cells, for example, to activate peripheral blood mononuclear cells (PBMCs) ex vivo, which are then infused (re-infused) into a subject.

In some embodiments, bacterial vaccine containing RNA (e.g., mRNA) polynucleotides as described herein can be administered to a subject (e.g., a mammalian subject, such as a human subject), and the RNA (e.g., mRNA) polynucleotides are translated in vivo to produce an antigenic polypeptide.

The bacterial RNA (e.g., mRNA) vaccines may be induced for translation of a polypeptide (e.g., antigen or immunogen) in a cell, tissue or organism. In some embodiments, such translation occurs in vivo, although such translation may occur ex vivo, in culture or in vitro. In some embodiments, the cell, tissue or organism is contacted with an effective amount of a composition containing a bacterial RNA (e.g., mRNA) vaccine that contains a polynucleotide that has at least one a translatable region encoding an antigenic polypeptide.

An “effective amount” of a bacterial RNA (e.g. mRNA) vaccine is provided based, at least in part, on the target tissue, target cell type, means of administration, physical characteristics of the polynucleotide (e.g., size, and extent of modified nucleosides) and other components of the vaccine, and other determinants. In general, an effective amount of the bacterial RNA (e.g., mRNA) vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

In some embodiments, RNA (e.g. mRNA) vaccines (including polynucleotides their encoded polypeptides) in accordance with the present disclosure may be used for treatment of bacterial infections.

Bacterial RNA (e.g. mRNA) vaccines may be administered prophylactically or therapeutically as part of an active immunization scheme to healthy individuals or early in infection during the incubation phase or during active infection after onset of symptoms. In some embodiments, the amount of RNA (e.g., mRNA) vaccine of the present disclosure provided to a cell, a tissue or a subject may be an amount effective for immune prophylaxis.

Bacterial RNA (e.g. mRNA) vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, bacterial RNA (e.g. mRNA) vaccines may be administered intramuscularly or intradermally, similarly to the administration of inactivated vaccines known in the art.

Bacterial RNA (e.g. mRNA) vaccines may be utilized in various settings depending on the prevalence of the infection or the degree or level of unmet medical need. As a non-limiting example, the RNA (e.g., mRNA) vaccines may be utilized to treat and/or prevent a variety of bacterial infections. RNA (e.g., mRNA) vaccines have superior properties in that they produce much larger antibody titers and produce responses early than commercially available anti-bacterial agents/compositions.

Provided herein are pharmaceutical compositions including bacterial RNA (e.g. mRNA) vaccines and RNA (e.g. mRNA) vaccine compositions and/or complexes optionally in combination with one or more pharmaceutically acceptable excipients.

Bacterial RNA (e.g. mRNA) vaccines may be formulated or administered alone or in conjunction with one or more other components. For instance, bacterial RNA (e.g., mRNA) vaccines (vaccine compositions) may comprise other components including, but not limited to, adjuvants.

In some embodiments, bacterial (e.g. mRNA) vaccines do not include an adjuvant (they are adjuvant-free).

Bacterial RNA (e.g. mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, bacterial RNA (e.g. mRNA) vaccines are administered to humans, human patients or subjects. For the purposes of the present disclosure, the phrase “active ingredient” generally refers to the RNA (e.g., mRNA) vaccines or the polynucleotides contained therein, for example, RNA polynucleotides (e.g., mRNA polynucleotides) encoding antigenic polypeptides.

Formulations of the bacterial vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Bacterial RNA (e.g. mRNA) vaccines can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with bacterial RNA (e.g. mRNA) vaccines (e.g., for transplantation into a subject), hyaluronidase, nanoparticle mimics and combinations thereof.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments, a vaccine includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments the RNA (e.g., mRNA) vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA (e.g., mRNA) vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA (e.g., mRNA) vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA (e.g., mRNA) vaccine may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, including (e.g., consisting of) a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA (e.g., mRNA) vaccine may have one or more AU-rich sequences removed. These sequences, sometimes referred to as AURES are destabilizing sequences found in the 3′UTR. The AURES may be removed from the RNA (e.g., mRNA) vaccines. Alternatively the AURES may remain in the RNA (e.g., mRNA) vaccine.

### Nanoparticle Formulations

The lipid nanoparticle (LNP) delivery is superior to other formulations including a protamine base approach described in the literature and no additional adjuvants are to be necessary. The use of LNPs enables the effective delivery of chemically modified or unmodified mRNA vaccines. Both modified and unmodified LNP formulated mRNA vaccines are superior to conventional vaccines by a significant degree. In some embodiments the mRNA vaccines of the invention are superior to conventional vaccines by a factor of at least 10 fold, 20 fold, 40 fold, 50 fold, 100 fold, 500 fold or 1,000 fold.

The fact that LNP formulations significantly enhance the effectiveness of mRNA vaccines, including chemically modified and unmodified mRNA vaccines provides a baseline formulation for the bacterial vaccines of the invention. The results presented herein demonstrate the unexpected superior efficacy of the mRNA vaccines formulated in LNP with mutated N-glycosylation sites.

In some embodiments, bacterial RNA (e.g. mRNA) vaccines are formulated in a nanoparticle. In some embodiments, bacterial RNA (e.g. mRNA) vaccines are formulated in a lipid nanoparticle. In some embodiments, bacterial RNA (e.g. mRNA) vaccines are formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, bacterial RNA (e.g., mRNA) vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a bacterial RNA (e.g. mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and compounds of formula I-IV; (iii) a sterol, e.g., cholesterol; and (iv) a PEG-lipid, e.g., PEG-DMG or PEG-cDMA, in a molar ratio of 20-60% cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and compounds of formula I-IV. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In some embodiments, lipid nanoparticle formulations include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 38.5% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 15% of the neutral lipid, 40% of the sterol, and 5% of the PEG or PEG-modified lipid on a molar basis.

In some embodiments, lipid nanoparticle formulations include 57.2% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), 7.5% of the neutral lipid, 31.5% of the sterol, and 3.5% of the PEG or PEG-modified lipid on a molar basis.

In some embodiments, lipid nanoparticle formulations consist essentially of a lipid mixture in molar ratios of 20-70% cationic lipid: 5-45% neutral lipid: 20-55% cholesterol: 0.5-15% PEG-modified lipid. In some embodiments, lipid nanoparticle formulations consist essentially of a lipid mixture in a molar ratio of 20-60% cationic lipid: 5-25% neutral lipid: 25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprises 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprises 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprises 55% of the cationic lipid L319, 10% of the non-cationic lipid DSPC, 2.5% of the PEG lipid PEG-DMG and 32.5% of the structural lipid cholesterol.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the bacterial RNA (e.g. mRNA) vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 hMPV), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Liposomes, Lipoplexes, and Lipid Nanoparticles

The RNA (e.g., mRNA) vaccines of the disclosure can be formulated using one or more liposomes, lipoplexes, or lipid nanoparticles. In some embodiments, pharmaceutical compositions of RNA (e.g., mRNA) vaccines include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, the RNA (e.g., mRNA) vaccine pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the cationic lipid may be a low molecular weight cationic lipid such as those described in U.S. Patent Application No. 20130090372, the contents of which are herein incorporated by reference in their entirety.

In some embodiments, the RNA (e.g., mRNA) vaccines may be formulated in a lipid vesicle, which may have crosslinks between functionalized lipid bilayers.

In some embodiments, the RNA (e.g., mRNA) vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the RNA (e.g., mRNA) vaccines may be formulated in a lipid-polycation complex, which may further include a non-cationic lipid such as, but not limited to, cholesterol or dioleoyl phosphatidylethanolamine (DOPE).

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the RNA (e.g., mRNA) vaccines may be formulated in a lipid nanoparticle.

In some embodiments, the RNA (e.g., mRNA) vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US2013/0150625); 2-amino-3-[(9Z)-octadec-9-en-1-yloxy]-2-{[(9Z)-octadec-9-en-1-yloxy]methyl}propan-1-ol (Compound 2 in US2013/0150625); 2-amino-3-[(9Z,12Z)-octadeca-9,12-dien-1-yloxy]-2-[(octyloxy)methyl]propan-1-ol (Compound 3 in US2013/0150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US2013/0150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and compounds of formula I-IV; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the formulation includes from about 5-25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the formulation includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and compounds of formula I-IV. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety)

In some embodiments, the formulations of the present disclosure include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, the formulations of the present disclosure include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, the formulations of the present disclosure include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, the formulations of the present disclosure include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In some embodiments, the formulations of the present disclosure include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In some embodiments, the formulations of the present disclosure include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In some embodiments, the formulations of the present disclosure include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), about 7.5% of the neutral lipid, about 31.5% of the sterol, and about 3.5% of the PEG or PEG-modified lipid on a molar basis.

In some embodiments, lipid nanoparticle formulation consists essentially of a lipid mixture in molar ratios of about 20-70% cationic lipid: 5-45% neutral lipid: 20-55% cholesterol: 0.5-15% PEG-modified lipid; more preferably in a molar ratio of about 20-60% cationic lipid: 5-25% neutral lipid: 25-55% cholesterol: 0.5-15% PEG-modified lipid.

In some embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Examples of lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-1 6, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl] pyrrolidine, (20Z)—N,N-dimethylheptacos-20-en-1 0-amine, (15Z)—N,N-dimethyleptacos-15-en-1 0-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-1 0-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl} dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[0S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the LNP formulations of the RNA (e.g., mRNA) vaccines may contain PEG-c-DOMG at 3% lipid molar ratio. In some embodiments, the LNP formulations of the RNA (e.g., mRNA) vaccines may contain PEG-c-DOMG at 1.5% lipid molar ratio.

In some embodiments, the pharmaceutical compositions of the RNA (e.g., mRNA) vaccines may include at least one of the PEGylated lipids described in International Publication No. WO2012/099755, the contents of which are herein incorporated by reference in their entirety.

In some embodiments, the LNP formulation may contain PEG-DMG 2000 (1,2-dimyristoyl-sn-glycero-3-phophoethanolamine-N-[methoxy(polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA (e.g., mRNA) vaccines, PNAS 2012; PMID: 22908294, the contents of each of which are herein incorporated by reference in their entirety).

The lipid nanoparticles described herein may be made in a sterile environment.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013/033438, the contents of which are herein incorporated by reference in its entirety.

The nanoparticle formulation may comprise a polymer conjugate. The polymer conjugate may be a water soluble conjugate. The polymer conjugate may have a structure as described in U.S. Patent Application No. 2013/0059360, the contents of which are herein incorporated by reference in its entirety. In some embodiments, polymer conjugates with the polynucleotides of the present disclosure may be made using the methods and/or segmented polymeric reagents described in U.S. Patent Application No. 2013/0072709, the contents of which are herein incorporated by reference in its entirety. In some embodiments, the polymer conjugate may have pendant side groups comprising ring moieties such as, but not limited to, the polymer conjugates described in U.S. Patent Publication No. US2013/0196948, the contents which are herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al. (Science 2013 339, 971-975), herein incorporated by reference in its entirety). As shown by Rodriguez et al., the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the RNA (e.g., mRNA) vaccines of the present disclosure are formulated in nanoparticles which comprise a conjugate to enhance the delivery of the nanoparticles of the present disclosure in a subject. The conjugate may be the CD47 membrane or the conjugate may be derived from the CD47 membrane protein, such as the “self” peptide described previously. In some embodiments, the nanoparticle may comprise PEG and a conjugate of CD47 or a derivative thereof. In some embodiments, the nanoparticle may comprise both the “self” peptide described above and the membrane protein CD47.

In some embodiments, the RNA (e.g., mRNA) vaccine pharmaceutical compositions comprise the polynucleotides of the present disclosure and a conjugate that may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Patent Publication No. US2013/0184443, the contents of which are herein incorporated by reference in their entirety.

The nanoparticle formulations may be a carbohydrate nanoparticle comprising a carbohydrate carrier and a RNA (e.g., mRNA) vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012/109121; the contents of which are herein incorporated by reference in their entirety).

Nanoparticle formulations of the present disclosure may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA (e.g., mRNA) vaccines within the central nervous system. As a non-limiting example nanoparticles comprising a hydrophilic coating and methods of making such nanoparticles are described in U.S. Patent Publication No. US2013/0183244, the contents of which are herein incorporated by reference in their entirety.

In some embodiments, the lipid nanoparticles of the present disclosure may be hydrophilic polymer particles. Non-limiting examples of hydrophilic polymer particles and methods of making hydrophilic polymer particles are described in U.S. Patent Publication No. US2013/0210991, the contents of which are herein incorporated by reference in their entirety.

In some embodiments, the lipid nanoparticles of the present disclosure may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbon.

In some embodiments, an immune response may be elicited by delivering a lipid nanoparticle which may include a nanospecies, a polymer and an immunogen. (U.S. Publication No. 2012/0189700 and International Publication No. WO2012/099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013/110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013/116804, the contents of which are herein incorporated by reference in their entirety. The polymeric material may additionally be irradiated. As a non-limiting example, the polymeric material may be gamma irradiated (see e.g., International App. No. WO2012/082165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013/012476, herein incorporated by reference in its entirety), and (poly(ethylene glycol))-(poly(propylene oxide))-(poly(ethylene glycol)) triblock copolymer (see e.g., U.S. Publication 2012/0121718 and U.S. Publication 2010/0003337 and U.S. Pat. No. 8,263,665, the contents of each of which are herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in their entirety). A non-limiting scalable method to produce nanoparticles which can penetrate human mucus is described by Xu et al. (see, e.g., J Control Release 2013, 170(2):279-86; the contents of which are herein incorporated by reference in their entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. See, e.g., U.S. Publication 2010/0215580 and U.S. Publication 2008/0166414 and U.S. Publication 2013/0164343; the contents of each of which are herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion, which may increase the delivery of the mucus penetrating lipid nanoparticles to the mucosal tissue.

In some embodiments, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013/110028, the contents of which are herein incorporated by reference in their entirety.

In some embodiments, in order to enhance the delivery through the mucosal barrier the RNA (e.g., mRNA) vaccine formulation may comprise or be a hypotonic solution. Hypotonic solutions were found to increase the rate at which mucoinert particles such as, but not limited to, mucus-penetrating particles, were able to reach the vaginal epithelial surface (see e.g., Ensign et al. Biomaterials 2013 34(28):6922-9, the contents of which are herein incorporated by reference in their entirety).

In some embodiments, the RNA (e.g., mRNA) vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132, the contents of each of which are incorporated herein by reference in their entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, the contents of each of which are incorporated herein by reference in their entirety). One example of passive targeting of formulations to liver cells includes the DLin-DMA, DLin-KC2-DMA and DLin-MC3-DMA-based lipid nanoparticle formulations, which have been shown to bind to apolipoprotein E and promote binding and uptake of these formulations into hepatocytes in vivo (Akinc et al. Mol Ther. 2010 18:1357-1364, the contents of which are incorporated herein by reference in their entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, the contents of each of which are incorporated herein by reference in their entirety).

In some embodiments, the RNA (e.g., mRNA) vaccine is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2 (8), pp 1696-1702; the contents of which are herein incorporated by reference in their entirety). As a non-limiting example, the SLN may be the SLN described in International Patent Publication No. WO2013/105101, the contents of which are herein incorporated by reference in their entirety. As another non-limiting example, the SLN may be made by the methods or processes described in International Patent Publication No. WO2013/105101, the contents of which are herein incorporated by reference in their entirety.

Liposomes, lipoplexes, or lipid nanoparticles may be used to improve the efficacy of polynucleotides directed protein production as these formulations may be able to increase cell transfection by the RNA (e.g., mRNA) vaccine; and/or increase the translation of encoded protein. One such example involves the use of lipid encapsulation to enable the effective systemic delivery of polyplex plasmid DNA (Heyes et al., Mol Ther. 2007 15:713-720; the contents of which are incorporated herein by reference in their entirety). The liposomes, lipoplexes, or lipid nanoparticles may also be used to increase the stability of the polynucleotide.

In some embodiments, the RNA (e.g., mRNA) vaccines of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the RNA (e.g., mRNA) vaccines may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012/131104 and WO2012/131106, the contents of each of which are incorporated herein by reference in their entirety).

In some embodiments, the RNA (e.g., mRNA) vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA (e.g., mRNA) vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA (e.g., mRNA) vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the RNA (e.g., mRNA) vaccine controlled release and/or targeted delivery formulation comprising at least one polynucleotide may comprise at least one PEG and/or PEG related polymer derivatives as described in U.S. Pat. No. 8,404,222, the contents of which are incorporated herein by reference in their entirety.

In some embodiments, the RNA (e.g., mRNA) vaccine controlled release delivery formulation comprising at least one polynucleotide may be the controlled release polymer system described in US2013/0130348, the contents of which are incorporated herein by reference in their entirety.

In some embodiments, the RNA (e.g., mRNA) vaccines of the present disclosure may be encapsulated in a therapeutic nanoparticle, referred to herein as “therapeutic nanoparticle RNA (e.g., mRNA) vaccines.” Therapeutic nanoparticles may be formulated by methods described herein and known in the art such as, but not limited to, International Pub Nos. WO2010/005740, WO2010/030763, WO2010/005721, WO2010/005723, WO2012/054923, U.S. Publication Nos. US2011/0262491, US2010/0104645, US2010/0087337, US2010/0068285, US2011/0274759, US2010/0068286, US2012/0288541, US2013/0123351 and US2013/0230567 and U.S. Pat. Nos. 8,206,747, 8,293,276, 8,318,208 and 8,318,211; the contents of each of which are herein incorporated by reference in their entirety. In some embodiments, therapeutic polymer nanoparticles may be identified by the methods described in US Pub No. US2012/0140790, the contents of which are herein incorporated by reference in their entirety.

In some embodiments, the therapeutic nanoparticle RNA (e.g., mRNA) vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present disclosure (see International Pub No. WO2010/075072 and US Pub Nos. US2010/0216804, US2011/0217377 and US2012/0201859, the contents of each of which are incorporated herein by reference in their entirety). In another non-limiting example, the sustained release formulation may comprise agents which permit persistent bioavailability such as, but not limited to, crystals, macromolecular gels and/or particulate suspensions (see U.S. Patent Publication No US2013/0150295, the contents of each of which are incorporated herein by reference in their entirety).

In some embodiments, the therapeutic nanoparticle RNA (e.g., mRNA) vaccines may be formulated to be target specific. As a non-limiting example, the therapeutic nanoparticles may include a corticosteroid (see International Pub. No. WO2011/084518, the contents of which are incorporated herein by reference in their entirety). As a non-limiting example, the therapeutic nanoparticles may be formulated in nanoparticles described in International Pub No. WO2008/121949, WO2010/005726, WO2010/005725, WO2011/084521 and US Pub No. US2010/0069426, US2012/0004293 and US2010/0104655, the contents of each of which are incorporated herein by reference in their entirety.

In some embodiments, a subset of compounds of Formula (I) includes those of Formula (IIa), (IIb), (IIc), or (IIe):

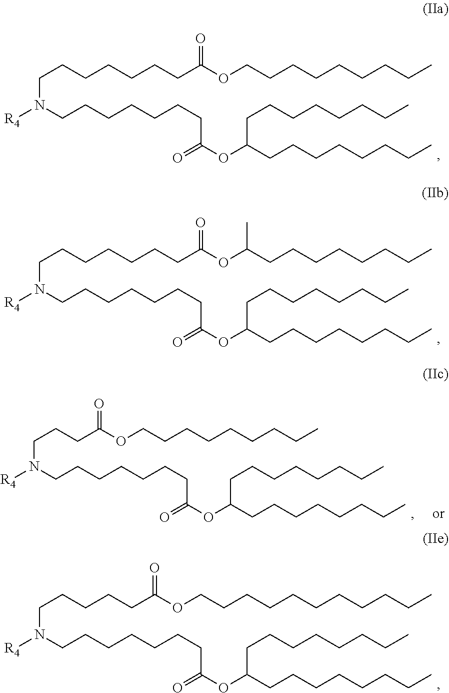

or a salt or isomer thereof, wherein R₄ is as described herein.

In some embodiments, a subset of compounds of Formula (I) includes those of Formula (IId):

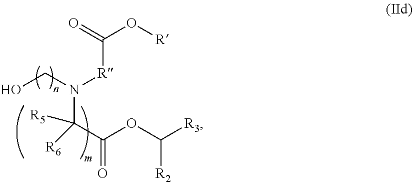

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

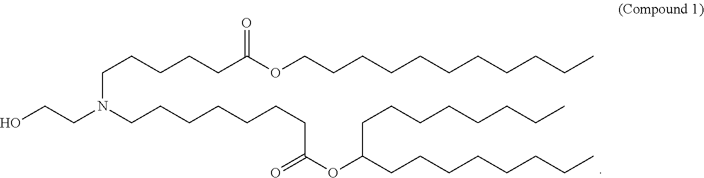 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

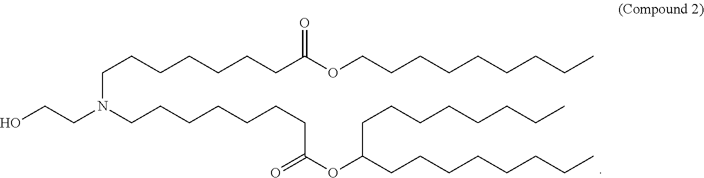 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, bras sicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Pharmaceutical Formulations

Provided herein are compositions (e.g., pharmaceutical compositions), methods, kits and reagents for prevention or treatment of bacterial infection(s) in humans and other mammals, for example. Bacterial RNA (e.g., mRNA) vaccines can be used as therapeutic or prophylactic agents. They may be used in medicine to prevent and/or treat infectious disease.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the disclosure features a pharmaceutical composition comprising a nanoparticle composition according to the preceding embodiments and a pharmaceutically acceptable carrier. For example, the pharmaceutical composition is refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition is a solution that is refrigerated for storage and/or shipment at, for example, about −20° C., −30° C., −40° C., −50° C., −60° C., −70° C., or −80° C.

In some embodiments, the disclosure provides a method of delivering a therapeutic and/or prophylactic (e.g., RNA, such as mRNA) to a cell (e.g., a mammalian cell). This method includes the step of administering to a subject (e.g., a mammal, such as a human) a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) a therapeutic and/or prophylactic, in which administering involves contacting the cell with the nanoparticle composition, whereby the therapeutic and/or prophylactic is delivered to the cell.

In some embodiments, the disclosure provides a method of producing a polypeptide of interest in a cell (e.g., a mammalian cell). The method includes the step of contacting the cell with a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) an mRNA encoding the polypeptide of interest, whereby the mRNA is capable of being translated in the cell to produce the polypeptide.

In some embodiments, the disclosure provides a method of treating a disease or disorder in a mammal (e.g., a human) in need thereof. The method includes the step of administering to the mammal a therapeutically effective amount of a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) a therapeutic and/or prophylactic (e.g., an mRNA). In some embodiments, the disease or disorder is characterized by dysfunctional or aberrant protein or polypeptide activity. For example, the disease or disorder is an infectious disease such as a bacterial infection.

An “effective amount” of a bacterial vaccine is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of bacterial vaccine provides an induced or boosted immune response as a function of antigen production in the cells of the subject. In some embodiments, an effective amount of the bacterial RNA vaccine containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

In some embodiments, the disclosure provides a method of delivering (e.g., specifically delivering) a therapeutic and/or prophylactic to a mammalian organ (e.g., a liver, spleen, lung, or femur). This method includes the step of administering to a subject (e.g., a mammal) a nanoparticle composition including (i) a lipid component including a phospholipid, a PEG lipid, a structural lipid, and a compound of Formula (I), (IA), (II), (IIa), (IIb), (IIc), (IId) or (IIe) and (ii) a therapeutic and/or prophylactic (e.g., an mRNA), in which administering involves contacting the cell with the nanoparticle composition, whereby the therapeutic and/or prophylactic is delivered to the target organ (e.g., a liver, spleen, lung, or femur).

In some embodiments, the disclosure features a method for the enhanced delivery of a therapeutic and/or prophylactic (e.g., an mRNA) to a target tissue (e.g., a liver, spleen, lung, or femur). This method includes administering to a subject (e.g., a mammal) a nanoparticle composition, the composition including (i) a lipid component including a compound of Formula (I), (IA), (II), (IIa), (IIb), (IIc), (IId) or (IIe), a phospholipid, a structural lipid, and a PEG lipid; and (ii) a therapeutic and/or prophylactic, the administering including contacting the target tissue with the nanoparticle composition, whereby the therapeutic and/or prophylactic is delivered to the target tissue.

In some embodiments, the disclosure features a method of lowering immunogenicity comprising introducing the nanoparticle composition of the disclosure into cells, wherein the nanoparticle composition reduces the induction of the cellular immune response of the cells to the nanoparticle composition, as compared to the induction of the cellular immune response in cells induced by a reference composition which comprises a reference lipid instead of a compound of Formula (I), (IA), (II), (IIa), (IIb), (IIc), (IId) or (IIe). For example, the cellular immune response is an innate immune response, an adaptive immune response, or both.

The disclosure also includes methods of synthesizing a compound of Formula (I), (IA), (II), (IIa), (IIb), (IIc), (IId) or (IIe) and methods of making a nanoparticle composition including a lipid component comprising the compound of Formula (I), (IA), (II), (IIa), (IIb), (IIc), (IId) or (IIe).

### Modes of Vaccine Administration

Bacterial RNA (e.g. mRNA) vaccines may be administered by any route which results in a therapeutically effective outcome. These include, but are not limited, to intradermal, intramuscular, and/or subcutaneous administration. The present disclosure provides methods comprising administering RNA (e.g., mRNA) vaccines to a subject in need thereof. The exact amount required will vary from subject to subject, depending on the species, age, and general condition of the subject, the severity of the disease, the particular composition, its mode of administration, its mode of activity, and the like. Bacterial RNA (e.g., mRNA) vaccines compositions are typically formulated in dosage unit form for ease of administration and uniformity of dosage. It will be understood, however, that the total daily usage of RNA (e.g., mRNA) vaccine compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Bacterial RNA (e.g., mRNA) vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, bacterial RNA vaccines may be administered intramuscularly, intranasally or intradermally, similarly to the administration of inactivated vaccines known in the art.

The bacterial RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including bacterial RNA vaccines and RNA vaccine compositions and/or complexes optionally in combination with one or more pharmaceutically acceptable excipients.

Bacterial RNA (e.g., mRNA) vaccines may be formulated or administered alone or in conjunction with one or more other components. For instance, bacterial RNA vaccines (vaccine compositions) may comprise other components including, but not limited to, adjuvants.

In some embodiments, bacterial RNA vaccines do not include an adjuvant (they are adjuvant free).

Bacterial RNA (e.g., mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, bacterial RNA vaccines are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with bacterial RNA vaccines (e.g., for transplantation into a subject), hyaluronidase, nanoparticle mimics and combinations thereof.

### Dosing/Administration

Provided herein are compositions (e.g., pharmaceutical compositions), methods, kits and reagents for prevention and/or treatment of bacterial infections in humans and other mammals. Bacterial RNA vaccines can be used as therapeutic or prophylactic agents. In some aspects, the RNA vaccines of the disclosure are used to provide prophylactic protection from bacterial infections. In some aspects, the RNA vaccines of the disclosure are used to treat a bacterial infection. In some embodiments, the bacterial vaccines of the present disclosure are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, the bacterial vaccines are administered to a subject (e.g., a mammalian subject, such as a human subject) in an effective amount to induce an antigen-specific immune response. The RNA encoding the bacterial antigen is expressed and translated in vivo to produce the antigen, which then stimulates an immune response in the subject.

Prophylactic protection from bacterial infections can be achieved following administration of a bacterial RNA vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against a bacterial infection is provided in aspects of the present disclosure. The method involves administering to the subject a bacterial RNA vaccine comprising at least one RNA (e.g., mRNA) having an open reading frame encoding at least one antigen, thereby inducing in the subject an immune response specific to the antigen, wherein anti-antigen antibody titer in the subject is increased following vaccination relative to anti-antigen antibody titer in a subject vaccinated with a prophylactically effective dose of a traditional vaccine against the bacterium. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, bacterial RNA (e.g. mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, bacterial RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, bacterial RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, bacterial RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a bacterial RNA (e.g., mRNA) vaccine composition may be administered three or four times.

In some embodiments, bacterial RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the bacterial RNA (e.g., mRNA) vaccine for use in a method of vaccinating a subject is administered to the subject as a single dosage of between 10 μg/kg and 400 μg/kg of the nucleic acid vaccine (in an effective amount to vaccinate the subject). In some embodiments the RNA (e.g., mRNA) vaccine for use in a method of vaccinating a subject is administered to the subject as a single dosage of between 10 μg and 400 μg of the nucleic acid vaccine (in an effective amount to vaccinate the subject). In some embodiments, a bacterial RNA (e.g., mRNA) vaccine for use in a method of vaccinating a subject is administered to the subject as a single dosage of 25-1000 μg (e.g., a single dosage of mRNA encoding bacterial antigen). In some embodiments, a bacterial RNA (e.g., mRNA) vaccine is administered to the subject as a single dosage of 25, 50, 100, 150, 200, 250, 300, 350, 400, 450, 500, 550, 600, 650, 700, 750, 800, 850, 900, 950 or 1000 μg. For example, a bacterial RNA (e.g., mRNA) vaccine may be administered to a subject as a single dose of 25-100, 25-500, 50-100, 50-500, 50-1000, 100-500, 100-1000, 250-500, 250-1000, or 500-1000 μg. In some embodiments, a bacterial RNA (e.g., mRNA) vaccine for use in a method of vaccinating a subject is administered to the subject as two dosages, the combination of which equals 25-1000 μg of the bacterial RNA (e.g., mRNA) vaccine.

A bacterial RNA (e.g. mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

Bacterial RNA (e.g., mRNA) Vaccine Formulations and Methods of Use

Some aspects of the present disclosure provide formulations of the bacterial RNA (e.g., mRNA) vaccine, wherein the RNA (e.g., mRNA) vaccine is formulated in an effective amount to produce an antigen specific immune response in a subject (e.g., production of antibodies specific to a bacterial antigenic polypeptide). “An effective amount” is a dose of an RNA (e.g., mRNA) vaccine effective to produce an antigen-specific immune response. Also provided herein are methods of inducing an antigen-specific immune response in a subject.

In some embodiments, the antigen-specific immune response is characterized by measuring an anti-Streptococcus, anti-Staphylococcus, and/or other bacterial antigenic polypeptide antibody titer produced in a subject administered a bacterial RNA (e.g., mRNA) vaccine as provided herein. An antibody titer is a measurement of the amount of antibodies within a subject, for example, antibodies that are specific to a particular antigen (e.g., an anti-Streptococcus, anti-Staphylococcus, and/or anti-bacterial antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the bacterial RNA (e.g., mRNA) vaccine.

In some embodiments, an anti-antigenic polypeptide (e.g., an anti-Streptococcus, anti-Staphylococcus, and/or anti-bacterial antigenic polypeptide) antibody titer produced in a subject is increased 1 log to 10 log following vaccination relative to anti-antigen antibody titer in a subject vaccinated with a prophylactically effective dose of a traditional vaccine against the bacterium or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the bacterium or an unvaccinated subject.

In some embodiments, the anti-antigenic polypeptide (e.g., an anti-Streptococcus, anti-Staphylococcus, and/or anti-bacterial antigenic polypeptide) antibody titer produced in a subject is increased at least 2 times relative to a control. For example, the anti-antigenic polypeptide antibody titer produced in a subject may be increased at least 3 times, at least 4 times, at least 5 times, at least 6 times, at least 7 times, at least 8 times, at least 9 times, or at least 10 times relative to a control. In some embodiments, the anti-antigenic polypeptide antibody titer produced in the subject is increased 2, 3, 4, 5, 6, 7, 8, 9, or 10 times relative to a control. In some embodiments, the anti-antigenic polypeptide antibody titer produced in a subject is increased 2-10 times relative to a control. For example, the anti-antigenic polypeptide antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-antigenic polypeptide (e.g., an anti-Streptococcus, anti-Staphylococcus, and/or anti-bacterial antigenic polypeptide) antibody titer produced in a subject who has not been administered a bacterial RNA (e.g., mRNA) vaccine of the present disclosure. In some embodiments, a control is an anti-antigenic polypeptide antibody titer produced in a subject who has been administered a live attenuated bacterial vaccine. An attenuated vaccine is a vaccine produced by reducing the virulence of a viable (live). An attenuated bacteria is altered in a manner that renders it harmless or less virulent relative to live, unmodified bacteria. In some embodiments, a control is an anti-antigenic polypeptide antibody titer produced in a subject administered inactivated Streptococcus, Staphylococcus, and/or other bacterial vaccine. In some embodiments, a control is an anti-antigenic polypeptide antibody titer produced in a subject administered a recombinant or purified bacterial protein vaccine. Recombinant protein vaccines typically include protein antigens that either have been produced in a heterologous expression system (e.g., bacteria or yeast) or purified from large amounts of the pathogenic organism. In some embodiments, a control is an anti-antigenic polypeptide antibody titer produced in a subject who has been administered a Streptococcus, Staphylococcus, and/or other bacterial vaccine.

A method of eliciting an immune response in a subject against a bacterium is provided in other aspects of the disclosure. The method involves administering to the subject a bacterial RNA vaccine comprising at least one RNA polynucleotide having an open reading frame encoding at least one bacterial antigen, thereby inducing in the subject an immune response specific to the bacterial antigen, wherein the immune response in the subject is equivalent to an immune response in a subject vaccinated with a traditional vaccine against the bacterial antigen at 2 times to 100 times the dosage level relative to the RNA vaccine.

In some embodiments, the immune response in the subject is equivalent to an immune response in a subject vaccinated with a traditional vaccine at twice the dosage level relative to the bacterial RNA vaccine. In some embodiments, the immune response in the subject is equivalent to an immune response in a subject vaccinated with a traditional vaccine at three times the dosage level relative to the bacterial RNA vaccine. In some embodiments, the immune response in the subject is equivalent to an immune response in a subject vaccinated with a traditional vaccine at 4 times, 5 times, 10 times, 50 times, or 100 times the dosage level relative to the bacterial RNA vaccine. In some embodiments, the immune response in the subject is equivalent to an immune response in a subject vaccinated with a traditional vaccine at 10 times to 1000 times the dosage level relative to the bacterial RNA vaccine. In some embodiments, the immune response in the subject is equivalent to an immune response in a subject vaccinated with a traditional vaccine at 100 times to 1000 times the dosage level relative to the bacterial RNA vaccine.

In other embodiments, the immune response is assessed by determining [protein] antibody titer in the subject. In other embodiments, the ability of serum or antibody from an immunized subject is tested for its ability to neutralize viral uptake or reduce the transformation of human B lymphocytes. In other embodiments, the ability to promote a robust T cell response(s) is measured using art recognized techniques.

Other aspects the disclosure provide methods of eliciting an immune response in a subject against a bacterium by administering to the subject a bacterial RNA vaccine comprising at least one RNA polynucleotide having an open reading frame encoding at least one bacterial antigen, thereby inducing in the subject an immune response specific to the bacterial antigen, wherein the immune response in the subject is induced 2 days to 10 weeks earlier relative to an immune response induced in a subject vaccinated with a prophylactically effective dose of a traditional vaccine against the bacterium. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a bacterium by administering to the subject a bacterial RNA vaccine having an open reading frame encoding a first antigen, wherein the RNA polynucleotide does not include a stabilization element, and wherein an adjuvant is not co-formulated or co-administered with the vaccine.

Bacterial RNA (e.g., mRNA) vaccines may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. Bacterial RNA (e.g., mRNA) vaccines compositions are typically formulated in dosage unit form for ease of administration and uniformity of dosage. It will be understood, however, that the total daily usage of bacterial RNA (e.g., mRNA) vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of a bacterial vaccine, as provided herein, may be as low as 20 μg, administered for example as a single dose or as two 10 μg doses. In some embodiments, the effective amount is a total dose of 20 μg-200 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg. In some embodiments, the effective amount is a total dose of 25 μg-200 μg. In some embodiments, the effective amount is a total dose of 50 μg-200 μg.

In some embodiments, bacterial RNA (e.g., mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, bacterial RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, bacterial RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, bacterial RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a bacterial RNA (e.g., mRNA) vaccine composition may be administered three or four times.

In some embodiments, bacterial RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the bacterial RNA (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments, the RNA vaccine for use in a method of vaccinating a subject is administered the subject a single dosage of between 10 μg and 400 μg of the nucleic acid vaccine in an effective amount to vaccinate the subject. In some embodiments, a bacterial RNA (e.g., mRNA) vaccine for use in a method of vaccinating a subject is administered to the subject as a single dosage of 25-1000 μg (e.g., a single dosage of mRNA encoding a bacterial antigen). In some embodiments, a bacterial RNA vaccine is administered to the subject as a single dosage of 25, 50, 100, 150, 200, 250, 300, 350, 400, 450, 500, 550, 600, 650, 700, 750, 800, 850, 900, 950 or 1000 μg. For example, a bacterial RNA vaccine may be administered to a subject as a single dose of 25-100, 25-500, 50-100, 50-500, 50-1000, 100-500, 100-1000, 250-500, 250-1000, or 500-1000 μg. In some embodiments, a bacterial RNA (e.g., mRNA) vaccine for use in a method of vaccinating a subject is administered to the subject as two dosages, the combination of which equals 25-1000 μg of the bacterial RNA (e.g., mRNA) vaccine.

A bacterial RNA (e.g., mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the bacterial RNA (e.g., mRNA) vaccine, wherein the bacterial RNA vaccine is formulated in an effective amount to produce an antigen specific immune response in a subject (e.g., production of antibodies specific to an antibacterial antigen). “An effective amount” is a dose of a bacterial RNA (e.g., mRNA) vaccine effective to produce an antigen-specific immune response. Also provided herein are methods of inducing an antigen-specific immune response in a subject.

As used herein, an immune response to a vaccine or LNP of the present invention is the development in a subject of a humoral and/or a cellular immune response to a (one or more) bacterial protein(s) present in the vaccine. For purposes of the present invention, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an antibacterial antigen antibody titer produced in a subject administered a bacterial RNA (e.g., mRNA) vaccine as provided herein. An antibody titer is a measurement of the amount of antibodies within a subject, for example, antibodies that are specific to a particular antigen (e.g., an antibacterial antigen) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the bacterial RNA (e.g., mRNA) vaccine.

In some embodiments, an antibacterial antigen antibody titer produced in a subject is increased by at least 1 log relative to a control. For example, antibacterial antigen antibody titer produced in a subject may be increased by at least 1.5, at least 2, at least 2.5, or at least 3 log relative to a control. In some embodiments, the antibacterial antigen antibody titer produced in the subject is increased by 1, 1.5, 2, 2.5 or 3 log relative to a control. In some embodiments, the antibacterial antigen antibody titer produced in the subject is increased by 1-3 log relative to a control. For example, the antibacterial antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the antibacterial antigen antibody titer produced in a subject is increased at least 2 times relative to a control. For example, the antibacterial antigen antibody titer produced in a subject may be increased at least 3 times, at least 4 times, at least 5 times, at least 6 times, at least 7 times, at least 8 times, at least 9 times, or at least 10 times relative to a control. In some embodiments, the antibacterial antigen antibody titer produced in the subject is increased 2, 3, 4, 5, 6, 7, 8, 9, or 10 times relative to a control. In some embodiments, the antibacterial antigen antibody titer produced in a subject is increased 2-10 times relative to a control. For example, the antibacterial antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the antibacterial antigen antibody titer produced in a subject who has not been administered a bacterial RNA (e.g., mRNA) vaccine. In some embodiments, a control is an antibacterial antigen antibody titer produced in a subject administered a recombinant or purified bacterial protein vaccine. Recombinant protein vaccines typically include protein antigens that either have been produced in a heterologous expression system (e.g., bacteria or yeast) or purified from large amounts of the pathogenic organism.

In some embodiments, the ability of a bacterial vaccine to be effective is measured in a murine model. For example, the bacterial vaccines may be administered to a murine model and the murine model assayed for induction of neutralizing antibody titers. Viral challenge studies may also be used to assess the efficacy of a vaccine of the present disclosure. For example, the bacterial vaccines may be administered to a murine model, the murine model challenged with a bacterium, and the murine model assayed for survival and/or immune response (e.g., neutralizing antibody response, T cell response (e.g., cytokine response)).

In some embodiments, an effective amount of a bacterial RNA (e.g., mRNA) vaccine is a dose that is reduced compared to the standard of care dose of a recombinant bacterial protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified bacterial protein vaccine, or a live attenuated or inactivated bacterial vaccine, or a bacterial VLP vaccine, that a physician/clinician or other medical professional would administer to a subject to treat or prevent a bacterial infectoin, or a bacteria-related condition, while following the standard of care guideline for treating or preventing a bacterial infection, or a bacteria-related condition.

In some embodiments, the antibacterial antigen antibody titer produced in a subject administered an effective amount of a bacterial RNA vaccine is equivalent to an antibacterial antigen antibody titer produced in a control subject administered a standard of care dose of a recombinant or purified bacterial protein vaccine, or a live attenuated or inactivated bacterial vaccine, or a bacterial VLP vaccine.

In some embodiments, an effective amount of a bacterial RNA (e.g., mRNA) vaccine is a dose equivalent to an at least 2-fold reduction in a standard of care dose of a recombinant or purified bacterial protein vaccine. For example, an effective amount of a bacterial RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified bacterial protein vaccine. In some embodiments, an effective amount of a bacterial RNA vaccine is a dose equivalent to an at least at least 100-fold, at least 500-fold, or at least 1000-fold reduction in a standard of care dose of a recombinant or purified bacterial protein vaccine. In some embodiments, an effective amount of a bacterial RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified bacterial protein vaccine. In some embodiments, the antibacterial antigen antibody titer produced in a subject administered an effective amount of a bacterial RNA vaccine is equivalent to an antibacterial antigen antibody titer produced in a control subject administered the standard of care dose of a recombinant protein or bacterial protein vaccine, or a live attenuated or inactivated bacterial vaccine, or a bacterial VLP vaccine. In some embodiments, an effective amount of a bacterial RNA (e.g., mRNA) vaccine is a dose equivalent to a 2-fold to 1000-fold (e.g., 2-fold to 100-fold, 10-fold to 1000-fold) reduction in the standard of care dose of a recombinant or purified bacterial protein vaccine, wherein the antibacterial antigen antibody titer produced in the subject is equivalent to an antibacterial antigen antibody titer produced in a control subject administered the standard of care dose of a recombinant or purified bacterial protein vaccine, or a live attenuated or inactivated bacterial vaccine, or a bacterial VLP vaccine.

In some embodiments, the effective amount of a bacterial RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant bacterial protein vaccine. In some embodiments, such as the foregoing, the antibacterial antigen antibody titer produced in the subject is equivalent to an anti bacterial antigen antibody titer produced in a control subject administered the standard of care dose of a recombinant or purified bacterial protein vaccine, or a live attenuated or inactivated bacterial vaccine, or a bacterial VLP vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820--, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant bacterial protein vaccine. In some embodiments, such as the foregoing, an antibacterial antigen antibody titer produced in the subject is equivalent to an antibacterial antigen antibody titer produced in a control subject administered the standard of care dose of a recombinant or purified bacterial protein vaccine, or a live attenuated or inactivated bacterial vaccine, or a bacterial VLP vaccine.

In some embodiments, the effective amount of a bacterial RNA (e.g., mRNA) vaccine is a total dose of 50-1000 μg. In some embodiments, the effective amount of a bacterial RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of a bacterial RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of a bacterial RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of a bacterial RNA (e.g., mRNA) vaccine is a total dose of 25, 50, 100, 150, 200, 250, 300, 350, 400, 450, or 500 μg administered to the subject a total of two times.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the bacterial vaccine is at least 60% relative to unvaccinated control subjects. For example, efficacy of the bacterial vaccine may be at least 65%, at least 70%, at least 75%, at least 80%, at least 85%, at least 95%, at least 98%, or 100% relative to unvaccinated control subjects.

Sterilizing Immunity.

Sterilizing immunity refers to a unique immune status that prevents effective pathogen infection into the host. In some embodiments, the effective amount of a bacterial vaccine of the present disclosure is sufficient to provide sterilizing immunity in the subject for at least 1 year. For example, the effective amount of a bacterial vaccine of the present disclosure is sufficient to provide sterilizing immunity in the subject for at least 2 years, at least 3 years, at least 4 years, or at least 5 years. In some embodiments, the effective amount of a bacterial vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen.

In some embodiments, the effective amount of a bacterial vaccine of the present disclosure is sufficient to produce detectable levels of bacterial antigen as measured in serum of the subject at 1-72 hours post administration.

Titer.

An antibody titer is a measurement of the amount of antibodies within a subject, for example, antibodies that are specific to a particular antigen (e.g., an antibacterial antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a bacterial vaccine of the present disclosure is sufficient to produce a 1,000-10,000 neutralizing antibody titer produced by neutralizing antibody against the bacterial antigen as measured in serum of the subject at 1-72 hours post administration. In some embodiments, the effective amount is sufficient to produce a 1,000-5,000 neutralizing antibody titer produced by neutralizing antibody against the bacterial antigen as measured in serum of the subject at 1-72 hours post administration. In some embodiments, the effective amount is sufficient to produce a 5,000-10,000 neutralizing antibody titer produced by neutralizing antibody against the bacterial antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an antibacterial antigen antibody titer produced in the subject is increased by at least 1 log relative to a control. For example, an antibacterial antigen antibody titer produced in the subject may be increased by at least 2, 3, 4, 5, 6, 7, 8, 9 or 10 log relative to a control.

In some embodiments, an antibacterial antigen antibody titer produced in the subject is increased at least 2 times relative to a control. For example, an antibacterial antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated bacterial vaccine, an inactivated bacterial vaccine, or a protein subunit bacterial vaccine.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

### EXAMPLES

### Example 1: Manufacture of Polynucleotides

According to the present disclosure, the manufacture of polynucleotides and/or parts or regions thereof may be accomplished utilizing the methods taught in International Publication WO2014/152027, entitled “Manufacturing Methods for Production of RNA Transcripts,” the contents of which is incorporated herein by reference in its entirety.

Purification methods may include those taught in International Publication WO2014/152030 and International Publication WO2014/152031, each of which is incorporated herein by reference in its entirety.

Detection and characterization methods of the polynucleotides may be performed as taught in International Publication WO2014/144039, which is incorporated herein by reference in its entirety.

Characterization of the polynucleotides of the disclosure may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Publication WO2014/144711 and International Publication WO2014/144767, the content of each of which is incorporated herein by reference in its entirety.

### Example 2: Chimeric Polynucleotide Synthesis

According to the present disclosure, two regions or parts of a chimeric polynucleotide may be joined or ligated using triphosphate chemistry. A first region or part of 100 nucleotides or less is chemically synthesized with a 5′ monophosphate and terminal 3′desOH or blocked OH, for example. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

For ligation methods, ligation with DNA T4 ligase, followed by treatment with DNase should readily avoid concatenation.

The entire chimeric polynucleotide need not be manufactured with a phosphate-sugar backbone. If one of the regions or parts encodes a polypeptide, then such region or part may comprise a phosphate-sugar backbone.

Ligation is then performed using any known click chemistry, orthoclick chemistry, solulink, or other bioconjugate chemistries known to those in the art.

### Synthetic Route

The chimeric polynucleotide may be made using a series of starting segments. Such segments include:

(a) a capped and protected 5′ segment comprising a normal 3′OH (SEG. 1)

(b) a 5′ triphosphate segment, which may include the coding region of a polypeptide and a normal 3′OH (SEG. 2)

(c) a 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) may be treated with cordycepin and then with pyrophosphatase to create the 5′ monophosphate.

Segment 2 (SEG. 2) may then be ligated to SEG. 3 using RNA ligase. The ligated polynucleotide is then purified and treated with pyrophosphatase to cleave the diphosphate. The treated SEG. 2-SEG. 3 construct may then be purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA may be performed using 2×KAPA HIFI™ HotStart ReadyMix by Kapa Biosystems (Woburn, Mass.). This system includes 2×KAPA ReadyMix 12.5 μl; Forward Primer (10 μM) 0.75 μl; Reverse Primer (10 μM) 0.75 μl; Template cDNA 100 ng; and dH₂0 diluted to 25.0 μl. The reaction conditions may be at 95° C. for 5 min. The reaction may be performed for 25 cycles of 98° C. for 20 sec, then 58° C. for 15 sec, then 72° C. for 45 sec, then 72° C. for 5 min, then 4° C. to termination.

The reaction may be cleaned up using Invitrogen's PURELINK™ PCR Micro Kit (Carlsbad, Calif.) per manufacturer's instructions (up to 5 μg). Larger reactions may require a cleanup using a product with a larger capacity. Following the cleanup, the cDNA may be quantified using the NANODROP™ and analyzed by agarose gel electrophoresis to confirm that the cDNA is the expected size. The cDNA may then be submitted for sequencing analysis before proceeding to the in vitro transcription reaction.

### Example 4: In Vitro Transcription (IVT)

The in vitro transcription reaction generates RNA polynucleotides. Such polynucleotides may comprise a region or part of the polynucleotides of the disclosure, including chemically modified RNA (e.g., mRNA) polynucleotides. The chemically modified RNA polynucleotides can be uniformly modified polynucleotides. The in vitro transcription reaction utilizes a custom mix of nucleotide triphosphates (NTPs). The NTPs may comprise chemically modified NTPs, or a mix of natural and chemically modified NTPs, or natural NTPs.

A typical in vitro transcription reaction includes the following:

| | | | |
|---|---|---|---|
| 1) | Template cDNA | 1.0 | μg |
| 2) | 10x transcription buffer | 2.0 | μl |
| | (400 mM Tris-HCl pH 8.0, | | |
| | 190 mM MgCl₂, 50 mM DTT, | | |
| | 10 mM Spermidine) | | |
| 3) | Custom NTPs (25 mM each) | 0.2 | μl |
| 4) | RNase Inhibitor | 20 | U |
| 5) | T7 RNA polymerase | 3000 | U |

| | | |
|---|---|---|
| 6) | dH₂0 | up to 20.0 μl. and |
| 7) | Incubation at 37° C. for 3 hr-5 hrs. | |

The crude IVT mix may be stored at 4° C. overnight for cleanup the next day. 1 U of RNase-free DNase may then be used to digest the original template. After 15 minutes of incubation at 37° C., the mRNA may be purified using Ambion's MEGACLEAR™ Kit (Austin, Tex.) following the manufacturer's instructions. This kit can purify up to 500 μg of RNA. Following the cleanup, the RNA polynucleotide may be quantified using the NanoDrop and analyzed by agarose gel electrophoresis to confirm the RNA polynucleotide is the proper size and that no degradation of the RNA has occurred.

### Example 5: Enzymatic Capping

Capping of a RNA polynucleotide is performed as follows where the mixture includes: IVT RNA 60 μg-180 μg and dH₂0 up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂0 (Up to 28 μl); and incubation at 37° C. for 30 minutes for 60 μg RNA or up to 2 hours for 180 μg of RNA.

The RNA polynucleotide may then be purified using Ambion's MEGACLEAR™ Kit (Austin, Tex.) following the manufacturer's instructions. Following the cleanup, the RNA may be quantified using the NANODROP™ (ThermoFisher, Waltham, Mass.) and analyzed by agarose gel electrophoresis to confirm the RNA polynucleotide is the proper size and that no degradation of the RNA has occurred. The RNA polynucleotide product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing capped IVT RNA (100 μl); RNase Inhibitor (20 U); 10× Tailing Buffer (0.5 M Tris-HCl (pH 8.0), 2.5 M NaCl, 100 mM MgCl₂) (12.0 μl); 20 mM ATP (6.0 μl); Poly-A Polymerase (20 U); dH₂0 up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase may be a recombinant enzyme expressed in yeast.

It should be understood that the processivity or integrity of the polyA tailing reaction may not always result in an exact size polyA tail. Hence, polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the present disclosure.

### Example 7: Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 8: Capping Assays

### Protein Expression Assay

Polynucleotides (e.g., mRNA) encoding a polypeptide, containing any of the caps taught herein, can be transfected into cells at equal concentrations. The amount of protein secreted into the culture medium can be assayed by ELISA at 6, 12, 24 and/or 36 hours post-transfection. Synthetic polynucleotides that secrete higher levels of protein into the medium correspond to a synthetic polynucleotide with a higher translationally-competent cap structure.

### Purity Analysis Synthesis

RNA (e.g., mRNA) polynucleotides encoding a polypeptide, containing any of the caps taught herein can be compared for purity using denaturing Agarose-Urea gel electrophoresis or HPLC analysis. RNA polynucleotides with a single, consolidated band by electrophoresis correspond to the higher purity product compared to polynucleotides with multiple bands or streaking bands. Chemically modified RNA polynucleotides with a single HPLC peak also correspond to a higher purity product. The capping reaction with a higher efficiency provides a more pure polynucleotide population.

### Cytokine Analysis

RNA (e.g., mRNA) polynucleotides encoding a polypeptide, containing any of the caps taught herein can be transfected into cells at multiple concentrations. The amount of pro-inflammatory cytokines, such as TNF-alpha and IFN-beta, secreted into the culture medium can be assayed by ELISA at 6, 12, 24 and/or 36 hours post-transfection. RNA polynucleotides resulting in the secretion of higher levels of pro-inflammatory cytokines into the medium correspond to a polynucleotides containing an immune-activating cap structure.

### Capping Reaction Efficiency

RNA (e.g., mRNA) polynucleotides encoding a polypeptide, containing any of the caps taught herein can be analyzed for capping reaction efficiency by LC-MS after nuclease treatment. Nuclease treatment of capped polynucleotides yield a mixture of free nucleotides and the capped 5′-5-triphosphate cap structure detectable by LC-MS. The amount of capped product on the LC-MS spectra can be expressed as a percent of total polynucleotide from the reaction and correspond to capping reaction efficiency. The cap structure with a higher capping reaction efficiency has a higher amount of capped product by LC-MS.

### Example 9: Agarose Gel Electrophoresis of Modified RNA or RT PCR Products

Individual RNA polynucleotides (200-400 ng in a 20 μl volume) or reverse transcribed PCR products (200-400 ng) may be loaded into a well on a non-denaturing 1.2% Agarose E-Gel (Invitrogen, Carlsbad, Calif.) and run for 12-15 minutes, according to the manufacturer protocol.

### Example 10: Nanodrop Modified RNA Quantification and UV Spectral Data

Chemically modified RNA polynucleotides in TE buffer (1 μl) are used for Nanodrop UV absorbance readings to quantitate the yield of each polynucleotide from a chemical synthesis or in vitro transcription reaction.

### Example 11: Formulation of Modified mRNA Using Lipidoids

RNA (e.g., mRNA) polynucleotides may be formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may be used as a starting point. After formation of the particle, polynucleotide is added and allowed to integrate with the complex. The encapsulation efficiency is determined using a standard dye exclusion assays.

### Example 12: Immunization of C57Bl/6 with Different Streptococcus pneumoniae Pneumolysoid mRNA

Six- to eight-week old female C57Bl/6 mice were used to study different Streptococcus pneumoniae pneumolysoid mRNA vaccines. The vaccines were created using pneumolysin and pneumolysin variants, including N-glycosylated mutants (NGM). The mice were immunized intramuscularly on day 0 and then given a booster dose three weeks later, on day 21. Blood samples were collected for serum three days prior to the first immunization, on day 20, and on approximately day 40-41. The groups are described in Table 5.

TABLE 5
Overview of Immunization Study Groups

| | | | | | Dose | | | MC3/ | |
|---|---|---|---|---|---|---|---|---|---|
| | | | | Dosage | Vol | 1^{st} | 2^{nd} | mRNA | |
| G# | Antigen | Route | n = | (ug) | (ul) | Dose | Dose | conc | |
| 1 | PlyD1 | IM | 5 | 10 | 50 | Day | Day | 0.2 | mg/ml |
| | | | | | | 0 | 21 | | |
| 2 | PlyD1 | IM | 5 | 2 | 50 | Day | Day | 0.04 | mg/ml |
| | | | | | | 0 | 21 | | |
| 3 | PlyD1_ | IM | 5 | 10 | 50 | Day | Day | 0.2 | mg/ml |
| | NGM | | | | | 0 | 21 | | |
| 4 | PlyD1_ | IM | 5 | 2 | 50 | Day | Day | 0.04 | mg/ml |
| | NGM | | | | | 0 | 21 | | |
| 5 | L460D | IM | 5 | 10 | 50 | Day | Day | 0.2 | mg/ml |
| | | | | | | 0 | 21 | | |
| 6 | L460D | IM | 5 | 2 | 50 | Day | Day | 0.04 | mg/ml |
| | | | | | | 0 | 21 | | |
| 7 | L460D_ | IM | 5 | 10 | 50 | Day | Day | 0.2 | mg/ml |
| | NGM | | | | | 0 | 21 | | |
| 8 | L460D_ | IM | 5 | 2 | 50 | Day | Day | 0.04 | mg/ml |
| | NGM | | | | | 0 | 21 | | |
| 9 | D205R | IM | 5 | 10 | 50 | Day | Day | 0.2 | mg/ml |
| | | | | | | 0 | 21 | | |
| 10 | D205R | IM | 5 | 2 | 50 | Day | Day | 0.04 | mg/ml |
| | | | | | | 0 | 21 | | |
| 11 | D205R_ | IM | 5 | 10 | 50 | Day | Day | 0.2 | mg/ml |
| | NGM | | | | | 0 | 21 | | |
| 12 | D205R_ | IM | 5 | 2 | 50 | Day | Day | 0.04 | mg/ml |
| | NGM | | | | | 0 | 21 | | |
| 13 | Tris- | IM | 5 | n/a | 50 | Day | Day | | |
| | Sucrose | | | | | 0 | 21 | | |

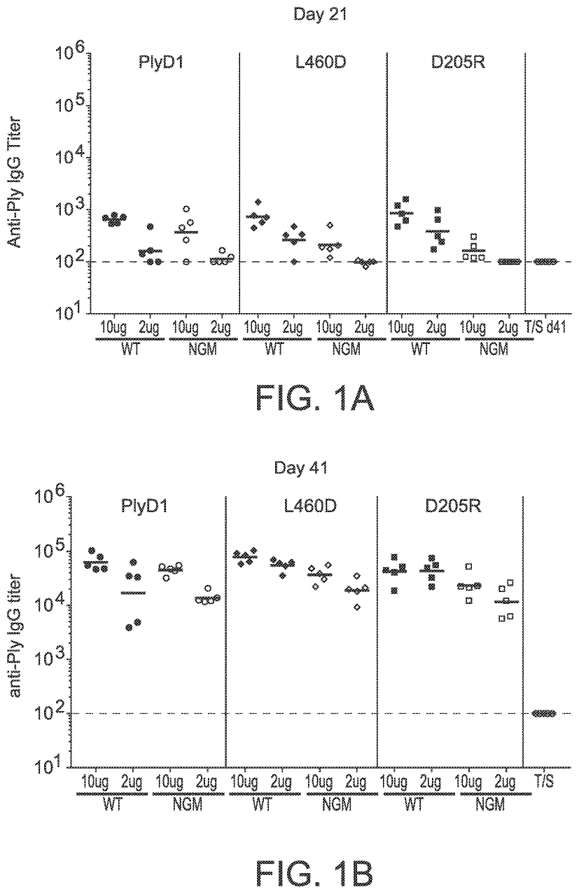

The serum samples were screened for anti-pneumolysin antibodies and penuemolysin neutralization. As shown in FIGS. 1A and 1B, the anti-pneumolysin IgG titers were roughly comparable or higher than the PlyD1 and L460D protein-based vaccines. Relative to the control, pneumolysoid mRNA elicited high serum IgG titers at both 21 days (the booster dose, FIG. 1A) and at 41 days (FIG. 1B). Mutating the N-glycosylation sites was shown to have a slight negative effect on the total IgG titers.

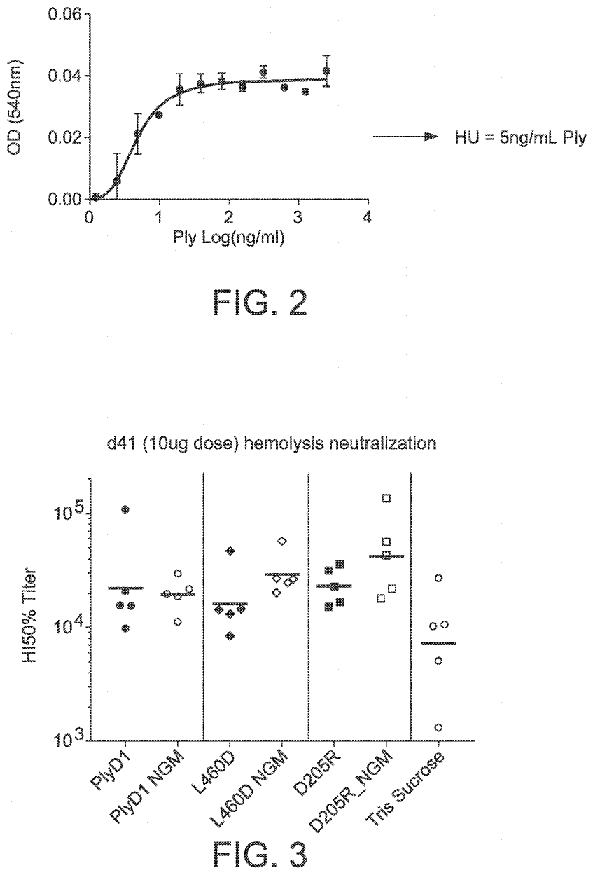

The serum samples were also used in an immune serum pneumolysin neutralization assay. In the assay, wild type pneumolysin and the serum samples were incubated together and then applied to red blood cells. Hemolysis of the red blood cells resulted in an OD of 540 nm, which was measured and quantified. The hemolytic unit (HU) determination (the pneumolysin concentration for 50% hemolysis) is given in FIG. 2. The HU was found to be 5 ng/mL of pneumolysin. FIG. 3 shows the results of the pneumolysin neutralization assay in the 10 μg dose group at 41 days. The pneumolysin neutralization assay was repeated using fresh red blood cells at the 2 μg and the 10 μg doses. The results are shown in FIG. 6. Mutating the N-glycosylation sites was shown to have a positive effect on neutralization activity.

### Example 13: Expression of mRNAs Encoding Pneumolysin, Characterized Pneumolysin Toxoids, and Novel Pneumolysin Toxoids

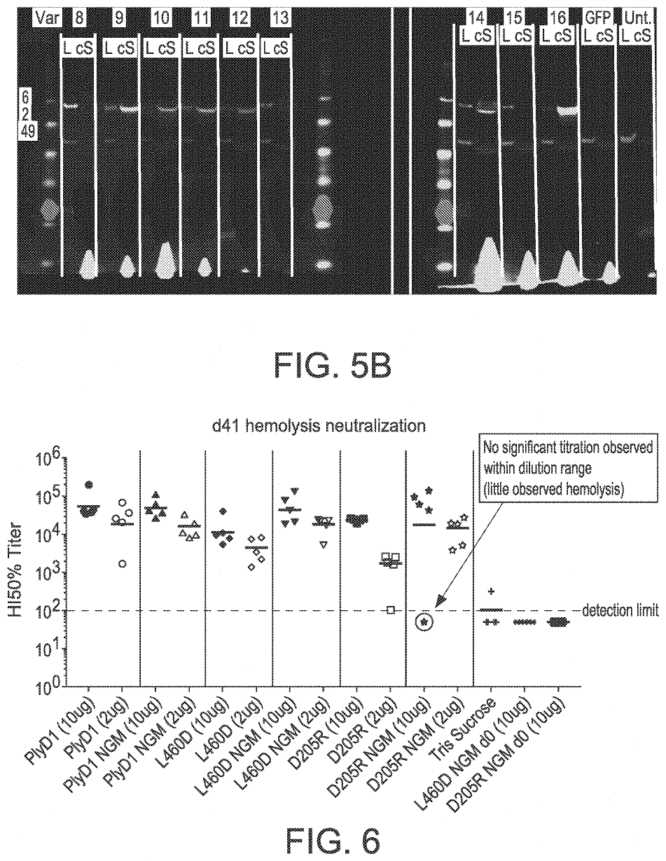

The expression levels of mRNAs encoding pneumolysin, characterized pneumolysin toxoids, and novel pneumolysin toxoid variants in HEK293F cells were determined. Samples were run on gels and stained with rabbit anti-His pAb (abcam ab9108) and mouse anti-beta actin mAb. Previously characterized pneumolysin toxoid variants are shown in FIG. 4 and novel pneumolysin toxoid variants are shown in FIGS. 5A-5B.

The expression in vitro expression levels of different mRNA constructs were measured as follows.

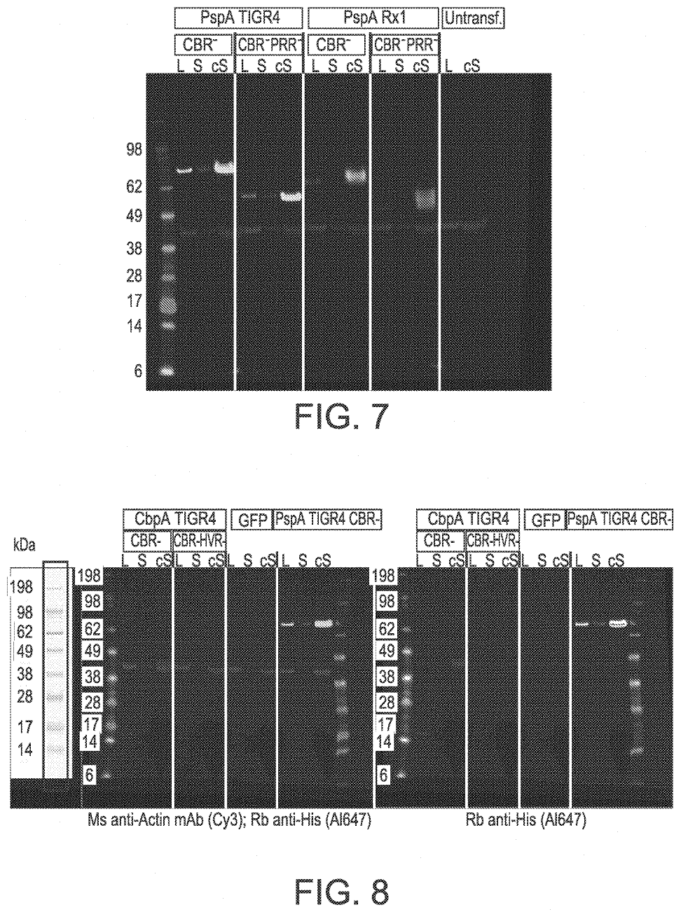

For pneumococcal surface protein A (PspA), which is serovariable (there are more than 40 serotypes), and necessary for virulence, constructs comprising one PspA from clades 1-2 and one PspA from clades 3-5, preferably containing one that elicits the most cross-protection, were used. The expression of S. pneumoniae PspA truncation variant mRNAs from TIGR4 and Rx1 strains in HEK293 cells are shown in FIG. 7 and in Table 6 below. Samples were run on gels and stained with rabbit anti-His antibody (A1647) and mouse anti-actin mAb (Cy3).

TABLE 6
Summary of PspA Data

| | Expected | | | | Obs. Size |
|---|---|---|---|---|---|
| | size | Obs. size | Obs. Size | Obs. Size | (concentrated |
| Name | (kDa) | (in vitro QC) | (cell lysate) | (cell sup.) | cell sup.) |
| Sp_PspA_noCBR_TIGR4_nlgK_cHis_no4A | 57.854 | 78.468 | ~70 | ~70 | ~70 |
| Sp_PspA_noCBR_noPRR_TIGR4_nlgK_cHis_no4A | 50.105 | 59.190 | ~55 | ~55 | ~55 |
| Sp_PspA_noCBR_Rx1_nlgK_cHis_no4A | 50.418 | 69.799 | ~62 | ~62+ | ~62-70 |
| | | | | (faint) | (glycosylated?) |
| Sp_PspA_noCBR_noPRR_Rx1_nlgK_cHis_no4A | 42.663 | 52.177 | ~50 | — | ~50-60 |
| | | | | | (glycosylated?) |

Similar procedures were used to determine the expression levels of constructs encoding choline-binding protein A (CbpA). The Western blot was performed using PspA as a control. Samples were run on gels and stained with rabbit anti-His antibody (A1647) and mouse anti-actin mAb (Cy3) or with only rabbit anti-His antibody (A1647). The results are shown in FIG. 8 and in Table 7 below.

Further, a liquid chromatograph-mass spectrometry (LC-MS/MS) analysis was performed to detect specific protein expression in the HEK293-F cell culture supernatant samples. Three samples (two transfected, and one untransfected control) were used and analyzed relative to two target proteins. Shotgun LC-MS/MS detected 46 peptides for both target proteins in the cell culture supernatants (46 peptides from Cbpa_aa1_477; 31 of which were shared with Cbpa_aa138-477).

TABLE 7
Summary of CbpA Data

| | Concentration | Expected MW | Observed MW |
|---|---|---|---|
| mRNA Name | (ng/ul) | (kD) | (kD) |
| Sp_Cbpa_aa1_477_nlgK_nHis | 898.6667 | 56.740 | 80.571 |
| Sp_Cbpa_aa138_477_nlgK_nHis | 1322.667 | 41.573 | 62.335 |
| AZ-eGFP-01-002 | 1233.566667 | | |

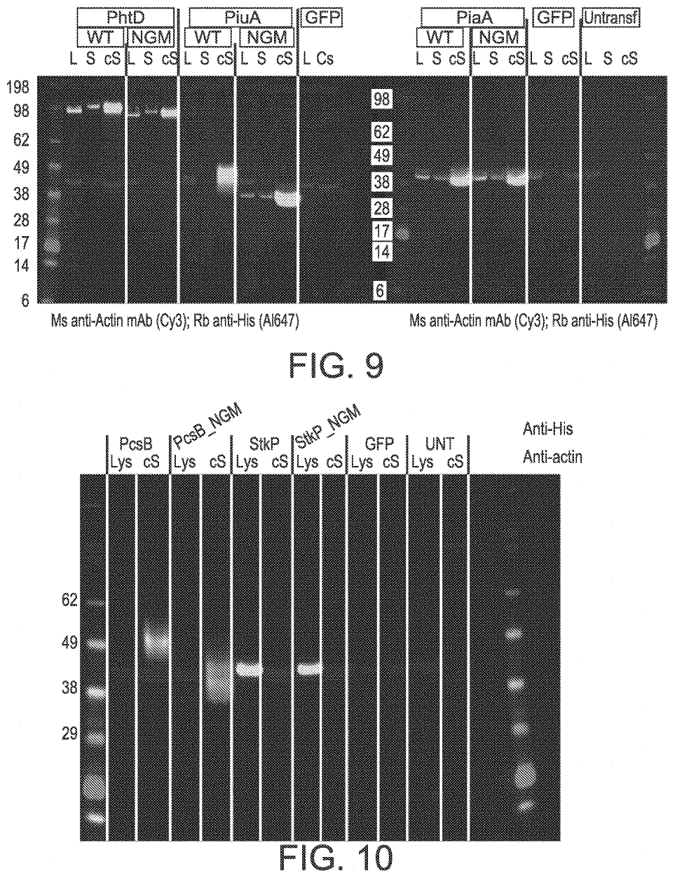

The expression levels of PhtD, PiuA, and PiaA were also examined. The expression levels of each in HEK293 cells are shown in FIG. 9 and in Table 8 below. Samples were run on gels and stained with rabbit anti-His antibody (A1647) and mouse anti-actin mAb (Cy3). All three were found to express the desired protein in all three sample (lysate, supernatant, and concentrated supernatant), indicating that both membrane-bound and secreted proteins were recognized. As shown in Table 8, all observed molecular weights (MW) were larger than the expected MW. Note that the wild-type variant is usually slightly larger than the N-glycosylation (NGM) mutant. Also, unspecific binding throughout the lysate samples was observed at approximately 15 kD. In the concentrated supernatant samples, unspecific binding was observed at approximately 96 kD.

TABLE 8
Summary of PhtD, PiuA, and PiaA Data

| | Final | | |
|---|---|---|---|
| | Concentration | Expected MW | Observed MW |
| mRNA Name | (ng/ul) | (kD) | (kD) |
| Sp_PhtD_nlgK_cHis | 612.60 | 94.762 | ~99 |
| Sp_PhtD_NGM_nlgK_cHis | 608.59 | 94.592 | ~97 |
| Sp_PiuA_nlgk_cHis | 784.08 | 34.546 | ~45 |
| Sp_PiuA_NGM_nlgk_cHis | 948.34 | 34.484 | ~38 |
| Sp_PiaA_nlgk_cHis | 1305.40 | 37.736 | ~41 |
| Sp_PiaA_NGM_nlgk_cHis | 1113.39 | 37.69 | ~41 |

Similarly, PcsB and StkP expression levels were examined using anti-His and anti-actin staining (FIG. 10 and Table 9). PcsB was found in the supernatant, but not the lysate, confirming that it is a secreted antigen. In contrast StkP was found in the lysate, but not the supernatant, indicating that it is a transmembrane protein. Note that the wild type and N-glycosylation mutant (NGM) appeared to be of the same size and intensity (FIG. 10). PcsB shows some glycosylation, possibly at different sites.

TABLE 9
Summary of PcsB and StkP Data

| | Expected | Observed | | Observed size |
|---|---|---|---|---|
| | size | QC size | Observed | concentrated |
| mRNA Name | (kDa) | (kDa) | size lysate | supernatant |
| Sp_PcsB_28_278_nlgK_cHis | 29.876 | 33.629 | — | ~50 |
| Sp_PcsB_28_278_NGM_nlgK_cHis | 29.800 | 33.167 | — | ~40 |
| Sp_StkP_345_659_nTMEM_cHis | 36.826 | 42.532 | ~45 | — |
| Sp_StkP_345_659_NGM_nTMEM_cHis | 36.750 | 42.721 | ~45 | — |

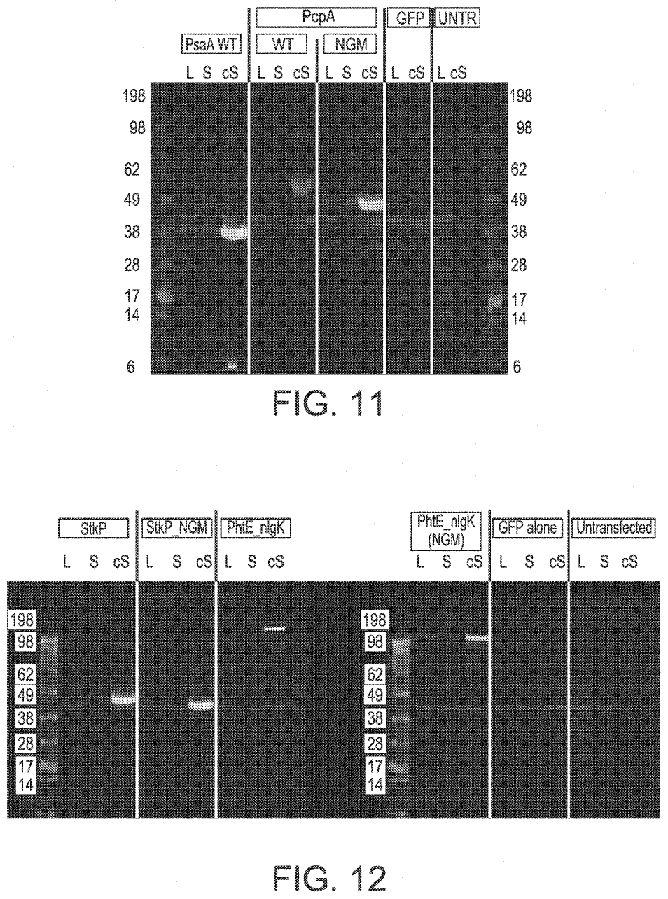

Similar experiments were carried out for PsaA and PcpA. The results are shown in FIG. 11 and in Table 10. Both appeared to be mainly present in the concentrated supernatant samples.

TABLE 10
Summary of PsaA and PcpA Data

| | Final | | | |
|---|---|---|---|---|
| | Concentration | Expected MW | QC MW | Observed MW |
| mRNA Name | (ng/ul) | (kD) | (kD) | (kD) |
| Sp_PsaA_nlgK_cHis | 1138.289 | 35.471 | 40.315 | ~39 |
| Sp_PcpA_nlgK_cHis | 1272.20653 | 51.541 | 56.044 | ~56 |
| Sp_PcpA_NGM_nlgK_cHis | 1346.492 | 51.477 | 53.878 | ~50 |
| Az-eGFP-01-002 | 1233.570 | — | — | — |

The expression levels of SktP and PhtE were also examined using similar methods. The results are shown in FIG. 12 and in Table 11. StkP and the StkP N-glycosylated mutant were found to express the desired protein in all three sample types (lysate, supernatant, and concentrated supernatant). The concentrated supernatant showed the strongest response. Both also had MWs that were larger than expected. PhtE_nIgK was found to only express in the concentrated supernatant sample with an accurate MW; however its N-glycosylated mutant (PhtE_nIgK_NGM) was found to express an accurate MW in all three samples. For both antigens, the N-glycosylated mutant was found to be slightly smaller than the wild type, and unspecific binding in the concentrated supernatant samples was found to be present at around 96 kDa.

TABLE 11
Summary of SktP and PhtE Data

| | Expected | QC | |
|---|---|---|---|
| | Molecular Weight | Measured MW | Observed MW |
| mRNA Name | (kD) | (kD) | (kD) |
| Sp_StkP_364_659_noTMEM_cHis | 34.76 | 29.45 | ~43 |
| Sp_StkP_364_659_noTMEM_NGM_cHis | 34.68 | 35.65 | ~42 |
| Sp_PhtE_nlgK_cHis | 115.21 | 138.72 | ~115 |
| Sp_PhtE_nlgK_NGM_cHis | 115.04 | 136.509 | ~112 |
| GFP | — | — | — |

### Example 14: Immunization of C57BL/6 with Different Streptococcus pneumoniae mRNAs

Experiments similar to those described in Example 11 were performed. Six- to eight-week old female C57Bl/6 mice were used to study different Streptococcus pneumoniae pneumolysoid mRNA vaccines. The vaccines were created encoding PspA fragments from strains TIGR4 or Rx1, including N-glycosylated mutants (NGM). The mice were immunized intramuscularly on day 0 and then given a booster dose three weeks later, on day 21. Blood samples were collected for serum three days prior to the first immunization, on day 20, and on day 42. The groups are described in Table 12.

TABLE 12
Overview of Immunization Study Groups (PspA)

| | | | | | Dose | | | MC3/ | |
|---|---|---|---|---|---|---|---|---|---|
| | | | | Dosage | Vol | 1^{st} | 2^{nd} | mRNA | |
| G# | Antigen | Route | n = | (ug) | (ul) | Dose | Dose | conc | |
| 1 | PspA_ | IM | 5 | 10 | 50 | Day | Day | 0.2 | mg/ml |
| | noCBR_ | | | | | 0 | 21 | | |
| | TIGR4 | | | | | | | | |
| 2 | PspA_ | IM | 5 | 2 | 50 | Day | Day | 0.04 | mg/ml |
| | noCBR_ | | | | | 0 | 21 | | |
| | TIGR4 | | | | | | | | |
| 3 | PspA_ | IM | 5 | 10 | 50 | Day | Day | 0.2 | mg/ml |
| | noCBR_ | | | | | 0 | 21 | | |
| | noPRR_ | | | | | | | | |
| | TIGR4 | | | | | | | | |
| 4 | PspA_ | IM | 5 | 2 | 50 | Day | Day | 0.04 | mg/ml |
| | noCBR_ | | | | | 0 | 21 | | |
| | noPRR_ | | | | | | | | |
| | TIGR4 | | | | | | | | |
| 5 | PspA_ | IM | 5 | 10 | 50 | Day | Day | 0.2 | mg/ml |
| | noCBR_ | | | | | 0 | 21 | | |
| | Rx1 | | | | | | | | |
| 6 | PspA_ | IM | 5 | 2 | 50 | Day | Day | 0.04 | mg/ml |
| | noCBR_ | | | | | 0 | 21 | | |
| | Rx1 | | | | | | | | |
| 7 | PspA_ | IM | 5 | 10 | 50 | Day | Day | 0.2 | mg/ml |
| | noCBR_ | | | | | 0 | 21 | | |
| | noPRR_ | | | | | | | | |
| | Rx1 | | | | | | | | |
| 8 | PspA_ | IM | 5 | 2 | 50 | Day | Day | 0.04 | mg/ml |
| | noCBR_ | | | | | 0 | 21 | | |
| | noPRR_ | | | | | | | | |
| | Rx1 | | | | | | | | |
| 9 | PspA_ | IM | 5 | 10 | 50 | Day | Day | 0.2 | mg/ml |
| | noCBR_ | | | | | 0 | 21 | | |
| | Rx1 _ | | | | | | | | |
| | NGM | | | | | | | | |
| 10 | PspA_ | IM | 5 | 2 | 50 | Day | Day | 0.04 | mg/ml |
| | noCBR_ | | | | | 0 | 21 | | |
| | Rx1_ | | | | | | | | |
| | NGM | | | | | | | | |
| 11 | PspA_ | IM | 5 | 10 | 50 | Day | Day | 0.2 | mg/ml |
| | noCBR_ | | | | | 0 | 21 | | |
| | noPRR_ | | | | | | | | |
| | Rx1_ | | | | | | | | |
| | NGM | | | | | | | | |
| 12 | PspA_ | IM | 5 | 2 | 50 | Day | Day | 0.04 | mg/ml |
| | noCBR_ | | | | | 0 | 21 | | |
| | noPRR_ | | | | | | | | |
| | Rx1_ | | | | | | | | |
| | NGM | | | | | | | | |
| 13 | Tris- | IM | 5 | n/a | 50 | Day | Day | | |
| | Sucrose | | | | | 0 | 21 | | |

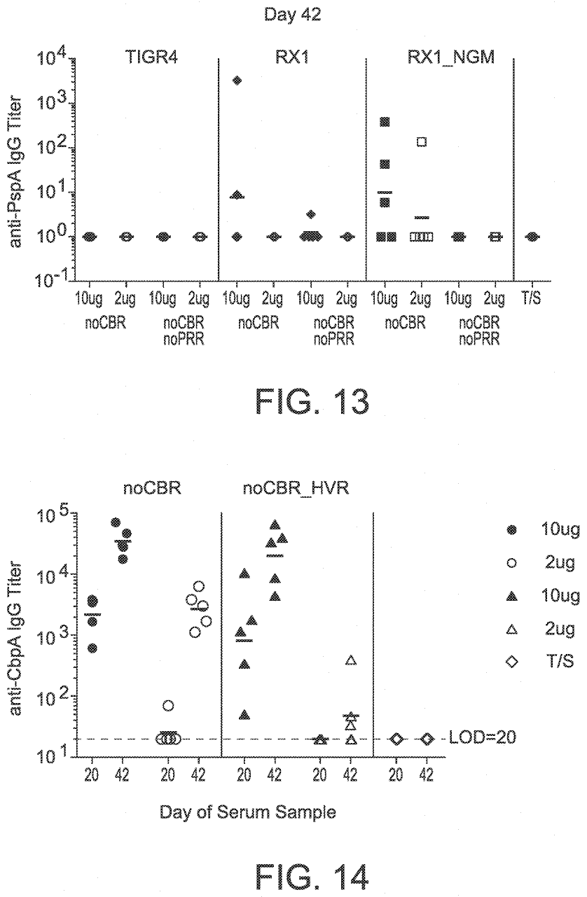

Groups 5, 7, 9, and 11 were expected to have high titers. The samples were screened for anti-PspA antibodies using an ELISA, and the results are shown in FIG. 13. Few samples showed positive titers, even with more concentrated serum samples.

The same study was repeated using CbpA variants. The groups are described in Table 13 below.

TABLE 13
Overview of Immunization Study Groups (CbpA)

| | | | | | Dose | | | MC3/ |
|---|---|---|---|---|---|---|---|---|
| | | | | Dosage | Vol | 1^{st} | 2^{nd} | mRNA |
| G# | Antigen | Route | n = | (ug) | (ul) | Dose | Dose | conc |
| 1 | CbpA_ | IM | 5 | 10 | 50 | Day | Day | 0.2 mg/ml |
| | noCBR_ | | | | | 0 | 21 | |
| | TIGR4 | | | | | | | |
| 2 | CbpA_ | IM | 5 | 2 | 50 | Day | Day | 0.04 mg/ml |
| | noCBR_ | | | | | 0 | 21 | |
| | TIGR4 | | | | | | | |
| 3 | CbpA_ | IM | 5 | 10 | 50 | Day | Day | 0.2 mg/ml |
| | noCBR_ | | | | | 0 | 21 | |
| | noHVR_ | | | | | | | |
| | TIGR4 | | | | | | | |
| 4 | CbpA_ | IM | 5 | 2 | 50 | Day | Day | 0.04 mg/ml |
| | noCBR_ | | | | | 0 | 21 | |
| | noHVR_ | | | | | | | |
| | TIGR4 | | | | | | | |
| 5 | Tris- | IM | 5 | n/a | 50 | Day | Day | |
| | Sucrose | | | | | 0 | 21 | |

The ELISA results are shown in FIG. 14. The “no CBR” group showed an increase in titer after the second dose: for the 10 μg dose, there was approximately a 11 og increase for the second dose; and for the 2 μg dose, there was an approximately 31 og increase. The “noCBR_HVR” groups also showed an increase in titer after the second dose: for the 10 μg dose, there was approximately a 21 og increase for the second dose; and for the 2 μg dose, there was an approximately 11 og increase after the second dose. However, there were two non-responders in the latter group. Note that the 10 μg dose was comparable for both fragments, while the 2 μg dose was much better for the no CBR fragment.

The experiment was also carried out to test different constructs encoding PiaA from the TIGR4 strain. The groups (n=5 mice per group) are shown in Table 14.

TABLE 14
Immunogenicity Study Groups (PiaA)

| | | | | | Dose | | | MC3/ |
|---|---|---|---|---|---|---|---|---|
| | | | | Dosage | Vol | 1^{st} | 2^{nd} | mRNA |
| G# | Antigen | Route | n = | (ug) | (ul) | Dose | Dose | conc |
| 1 | PiaA | IM | 5 | 10 | 50 | Day | Day | 0.2 mg/ml |
| | | | | | | 0 | 21 | |
| 2 | PiaA | IM | 5 | 2 | 50 | Day | Day | 0.04 mg/ml |
| | | | | | | 0 | 21 | |
| 3 | PiaA_ | IM | 5 | 10 | 50 | Day | Day | 0.2 mg/ml |
| | NGM | | | | | 0 | 21 | |
| 4 | PiaA_ | IM | 5 | 2 | 50 | Day | Day | 0.04 mg/ml |
| | NGM | | | | | 0 | 21 | |
| 5 | Tris- | IM | 5 | n/a | 50 | Day | Day | |
| | Sucrose | | | | | 0 | 21 | |

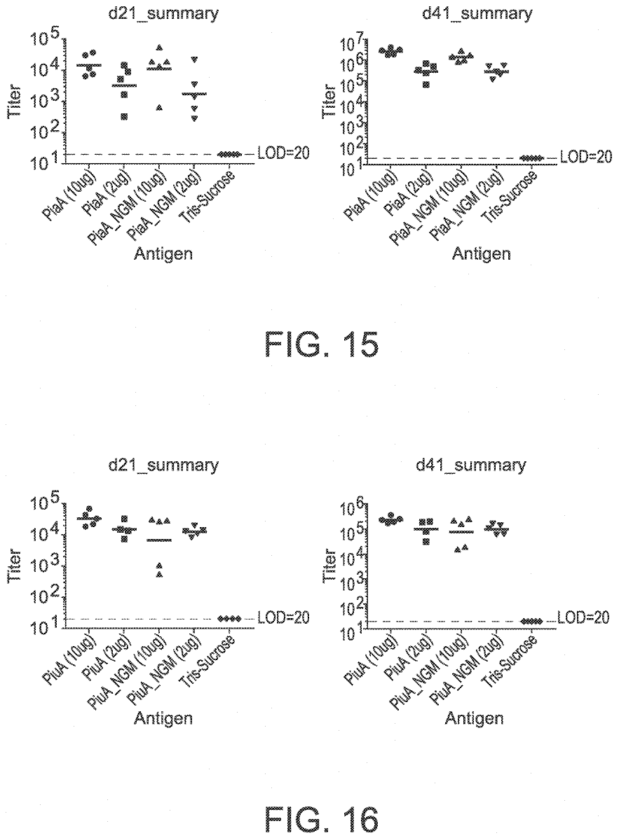

The ELISA results are shown in FIG. 15. A dose response was seen in both the wild type and the N-glycosylated mutants assayed. There was approximately a 21 og increase for all immunization groups after the boost, which was similar among groups (Day 42). However, the prime response was found to be more variable (Day 21). Note that the wild type and N-glycosylation mutants appear to be comparable.

The experiment was also performed to test different constructs encoding PiuA from the TIGR4 strain. The groups (n=5 mice per group) are shown in Table 15.

TABLE 15
Immunogenicity Study Groups (PiuA)

| | | | | | Dose | | | MC3/ |
|---|---|---|---|---|---|---|---|---|
| | | | | Dosage | Vol | 1^{st} | 2^{nd} | mRNA |
| G# | Antigen | Route | n = | (ug) | (ul) | Dose | Dose | conc |
| 1 | PiuA | IM | 5 | 10 | 50 | Day | Day | 0.2 mg/ml |
| | | | | | | 0 | 21 | |
| 2 | PiuA | IM | 5 | 2 | 50 | Day | Day | 0.04 mg/ml |
| | | | | | | 0 | 21 | |
| 3 | PiuA_ | IM | 5 | 10 | 50 | Day | Day | 0.2 mg/ml |
| | NGM | | | | | 0 | 21 | |
| 4 | PiuA_ | IM | 5 | 2 | 50 | Day | Day | 0.04 mg/ml |
| | NGM | | | | | 0 | 21 | |
| 5 | Tris- | IM | 5 | n/a | 50 | Day | Day | |
| | Sucrose | | | | | 0 | 21 | |

The results are shown in FIG. 16. Like PiaA, PiuA is a lipoprotein component of the iron ABC transporter and is highly conserved. Together, they have found to be protective in a mouse model of sepsis and pneumonia. As shown in FIG. 16, a dose response was seen in the wild type PiuA constructs, while a reverse dose response was observed in the N-glycosylated mutant constructs. There was about a 11 og increase for all immunizations following the boost, although the wild type constructs seemed to do slightly better than the NGM versions. Note that initial prime titers were high.

A similar experiment was performed using PhtD mRNA. Six- to eight-week old female C57Bl/6 mice were used to study different Streptococcus pneumoniae PhtD mRNA vaccines. The mice were immunized intramuscularly on day 1 and then given a booster dose three weeks later, on day 22. Blood samples were collected for serum three days prior to the first immunization, on day 22, and on day 36. On day 29, five mice per group were euthanized for T cell analysis (via splenic samples). The groups are described in Table 16.

TABLE 16
Immunogenicity Study Groups (PhtD)

| | | | | | Dose | | | MC3/ |
|---|---|---|---|---|---|---|---|---|
| | | | | Dosage | Vol | 1^{st} | 2^{nd} | mRNA |
| G# | Antigen | Route | n = | (ug) | (ul) | Dose | Dose | conc |
| 1 | PhtD | IM | 10 | 10 | 50 | Day | Day | 0.2 mg/ml |
| | | | | | | 1 | 22 | |
| 2 | PhtD | IM | 10 | 2 | 50 | Day | Day | 0.04 mg/ml |
| | | | | | | 1 | 22 | |
| 3 | PhtD_ | IM | 10 | 10 | 50 | Day | Day | 0.2 mg/ml |
| | NGM | | | | | 1 | 22 | |
| 4 | PhtD_ | IM | 10 | 2 | 50 | Day | Day | 0.04 mg/ml |
| | NGM | | | | | 1 | 22 | |
| 5 | Tris- | IM | 10 | n/a | 50 | Day | Day | |
| | Sucrose | | | | | 1 | 22 | |

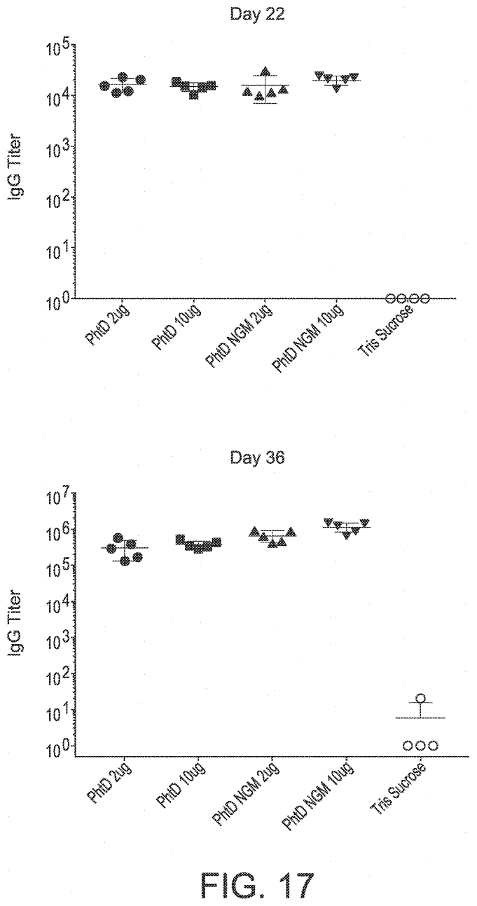

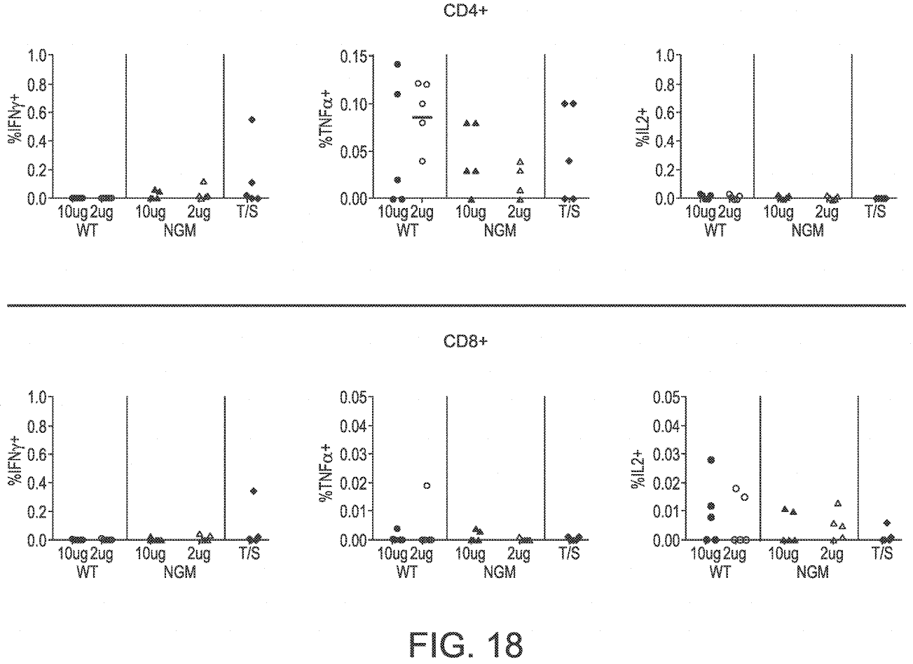

Anti-PhtD antibody titers were measured in the serum samples (FIG. 17). While a dose response was not observed, an increase of at least 11 og was seen in the titers following the boost dose. CD4+ T cell activation was determined from the splenic samples (FIG. 18). While a slight CD8 IL-2 response was seen, its percentage was very low. No response above the background level of activation was observed in any other the screens.

### Sequences

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 54)
GGGAAAUAAGAGAGAAAAGAAGAGUAAGAAGAAAUAUAAGAGCCACC
5′ UTR:
(SEQ ID NO: 69)
CAAGCUUUUGGACCCUCGUACAGAAGCUAAUACGACUCACUAUAGGGAAA
UAAGAGAGAAAAGAAGAGUAAGAAGAAAUAUAAGAGCCACC
3′ UTR:
(SEQ ID NO: 55)
UGAUAAUAGGCUGGAGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC

TABLE 1
Pneumolysin Nucleic Acid Sequences
Description Sequence SEQ ID NO:

S. pneumoniae pneumolysin gene, complete cds; Accession No. M17717:
TTACAAGACCAACCTTGATTGACTTAGATAAGGTATTTATGTTGGATAATACGGTTATTCCGACTTCTTATCTAGCCAGACGGC
GACGCAATGTCTCAGAAGAATTGTACGAGGAAATTTTGGATCACTTAGTCCAACCACGGCTGATTTCGCTGAACAAGTCTGAGT
TTATGCAACTCAATCCAGGAACTTATTAGGAGGAGAAGATGGCAAATAAAGCAGTAAATGACTTTATACTAGCTATGAATTACG
ATAAAAAGAAACTCTTGACCCATCAGGGAGAAAGTATTGAAAATCGTTTCATCAAAGAGGGTAATCAGCTACCCGATGAGTTTG
TTGTTATCGAAAGAAAGAAGCGGAGCTTGTCGACAAATACAAGTGATATTTCTGTAACAGCTACCAACGACAGTCGCCTCTATC
CTGGAGCACTTCTCGTAGTGGATGAGACCTTGTTAGAGAATAATCCCACTCTTCTTGCGGTTGATCGTGCTCCGATGACTTATA
GTATTGATTTGCCTGGTTTGGCAAGTAGCGATAGCTTTCTCCAAGTGGAAGACCCCAGCAATTCAAGTGTTCGCGGAGCGGTAA
ACGATTTGTTGGCTAAGTGGCATCAAGATTATGGTCAGGTCAATAATGTCCCAGCTAGAATGCAGTATGAAAAAATAACGGCTC
ACAGCATGGAACAACTCAAGGTCAAGTTTGGTTCTGACTTTGAAAAGACAGGGAATTCTCTTGATATTGATTTTAACTCTGTCC
ATTCAGGTGAAAAGCAGATTCAGATTGTTAATTTTAAGCAGATTTATTATACAGTCAGCGTAGACGCTGTTAAAAATCCAGGAG
ATGTGTTTCAAGATACTGTAACGGTAGAGGATTTAAAACAGAGAGGAATTTCTGCAGAGCGTCCTTTGGTCTATATTTCGAGTG
TTGCTTATGGGCGCCAAGTCTATCTCAAGTTGGAAACCACGAGTAAGAGTGATGAAGTAGAGGCTGCTTTTGAAGCTTTGATAA
AAGGAGTCAAGGTAGCTCCTCAGACAGAGTGGAAGCAGATTTTGGACAATACAGAAGTGAAGGCGGTTATTTTAGGGGGCGACC
CAAGTTCGGGTGCCCGAGTTGTAACAGGCAAGGTGGATATGGTAGAGGACTTGATTCAAGAAGGCAGTCGCTTTACAGCAGATC
ATCCAGGCTTGCCGATTTCCTATACAACTTCTTTTTTACGTGACAATGTAGTTGCGACCTTTCAAAACAGTACAGACTATGTTG
AGACTAAGGTTACAGCTTACAGAAACGGAGATTTACTGCTGGATCATAGTGGTGCCTATGTTGCCCAATATTATATTACTTGGG
ATGAATTATCCTATGATCATCAAGGTAAGGAAGTCTTGACTCCTAAGGCTTGGGACAGAAATGGGCAGGATTTGACGGCTCACT
TTACCACTAGTATTCCTTTAAAAGGGAATGTTCGTAATCTCTCTGTCAAAATTAGAGAGTGTACCGGGCTTGCCTGGGAATGGT
GGCGTACGGTTTATGAAAAAACCGATTTGCCACTAGTGCGTAAGCGGACGATTTCTATTTGGGGAACAACTCTCTATCCTCAGG
TAGAGGATAAGGTAGAAAATGACTAGGAGAGGAGAATGCTTGCGACAAAAAGA (SEQ ID NO: 1)
Sp_Ply_D205R_NGM_nIgK (5′UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCGACACCAGCGACATCAGCG
TGACCGCCACCAACGACGCCAGACTGTATCCTGGCGCTCTGCTGGTGGTGGACGAGACACTGCTGGAAAACAACCCCATCCTGC
TGGCCGTGGACAGAGCCCCCATGACCTACAGCATCGACCTGCCTGGCCTGGCCAGCAGCGATAGCTTTCTGCAGGTGGAAGATC
CCAGCAACAGCGCCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGCGGGCCGTGAAGAACCCTGGGGACGTGTTCCAGGATACCGTGACCGTGGAAGATCTGAAGCAGCGGGGCATCAGCG
CCGAGAGGCCACTGGTGTACATCAGCAGCGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGGGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACTCCGCCGACTACGTGGAAACAAAAGTGACCGCCTACCGGAACGGCGATCTGCTGCTGGATCACTCCGGCG
CCTATGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAGTGCACCGGACTGGCCTGGGAGTGGTGGCGGACCGTGTACGAAAAGACCGACCTGCCCCTCGTGCGGAAGCGGACCATCT
CTATCTGGGGCACCACGCTGTATCCTCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 2)
SP_Ply_L460D_NGM_nIgK (5′UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCGACACCAGCGACATCAGCG
TGACCGCCACCAACGACGCCAGACTGTATCCTGGCGCTCTGCTGGTGGTGGACGAGACACTGCTGGAAAACAACCCCATCCTGC
TGGCCGTGGACAGAGCCCCCATGACCTACAGCATCGACCTGCCTGGCCTGGCCAGCAGCGATAGCTTTCTGCAGGTGGAAGATC
CCAGCAACAGCGCCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGGACGCCGTGAAGAACCCCGGGGACGTGTTCCAGGATACCGTGACCGTGGAAGATCTGAAGCAGCGGGGCATCAGCG
CCGAGAGGCCACTGGTGTACATCAGCAGCGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGGGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACTCCGCCGACTACGTGGAAACAAAAGTGACCGCCTACCGGAACGGCGATCTGCTGCTGGATCACTCCGGCG
CCTATGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAGTGCACCGGACTGGCCTGGGAGTGGTGGCGGACCGTGTACGAAAAGACCGACCTGCCCCTCGTGCGGAAGCGGACCATCT
CTATCTGGGGCACCACCGACTACCCCCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 3)
SP_Ply_T65C_G293C_C428A_PlyD1_ NGM_nIgK (5′UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCGACACCAGCGACATCAGCG
TGACCGCCTGCAACGACGCCAGACTGTATCCTGGCGCTCTGCTGGTGGTGGACGAGACACTGCTGGAAAACAACCCCATCCTGC
TGGCCGTGGACAGAGCCCCCATGACCTACAGCATCGACCTGCCTGGCCTGGCCAGCAGCGATAGCTTTCTGCAGGTGGAAGATC
CCAGCAACAGCGCCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGGACGCCGTGAAGAACCCCGGGGACGTGTTCCAGGATACCGTGACCGTGGAAGATCTGAAGCAGCGGGGCATCAGCG
CCGAGAGGCCACTGGTGTACATCAGCAGCGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGTGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACTCCGCCGACTACGTGGAAACAAAAGTGACAGCCTACCGGAACGGCGATCTGCTGCTGGATCACTCCGGCG
CCTATGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAAGCCACCGGACTGGCCTGGGAGTGGTGGCGGACAGTGTACGAAAAGACCGACCTGCCCCTCGTGCGGAAGCGGACCATCT
CTATCTGGGGCACCACGCTGTATCCTCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 4)
SP_Ply_D205R_nIgK (5′ UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCAACACCAGCGACATCAGCG
TGACCGCCACCAACGACAGCAGACTGTATCCTGGCGCCCTGCTGGTGGTGGACGAGACACTGCTGGAAAACAACCCCACCCTGC
TGGCCGTGGACAGAGCCCCTATGACCTACAGCATCGACCTGCCTGGCCTGGCCAGCAGCGATAGCTTTCTGCAGGTGGAAGATC
CCAGCAACAGCAGCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGCGGGCCGTGAAGAACCCTGGGGACGTGTTCCAGGATACCGTGACCGTGGAAGATCTGAAGCAGCGGGGCATCAGCG
CCGAGAGGCCACTGGTGTACATCAGCTCTGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGGGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACAGCACCGACTACGTGGAAACAAAAGTGACCGCCTACCGGAACGGCGATCTGCTGCTGGATCACTCCGGCG
CCTACGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAGTGCACCGGACTGGCCTGGGAGTGGTGGCGGACCGTGTACGAAAAGACCGACCTGCCCCTCGTGCGGAAGCGGACCATCT
CTATCTGGGGCACCACGCTGTATCCTCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 30)
SP_Ply_L460D_nIgK (5′ UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCAACACCAGCGACATCAGCG
TGACCGCCACCAACGACAGCAGACTGTATCCTGGCGCCCTGCTGGTGGTGGACGAGACACTGCTGGAAAACAACCCCACCCTGC
TGGCCGTGGACAGAGCCCCTATGACCTACAGCATCGACCTGCCTGGCCTGGCCAGCAGCGATAGCTTTCTGCAGGTGGAAGATC
CCAGCAACAGCAGCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGGACGCCGTGAAGAACCCCGGGGACGTGTTCCAGGATACCGTGACCGTGGAAGATCTGAAGCAGCGGGGCATCAGCG
CCGAGAGGCCACTGGTGTACATCAGCTCTGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGGGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACAGCACCGACTACGTGGAAACAAAAGTGACCGCCTACCGGAACGGCGATCTGCTGCTGGATCACTCCGGCG
CCTACGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAGTGCACCGGACTGGCCTGGGAGTGGTGGCGGACCGTGTACGAAAAGACCGACCTGCCCCTCGTGCGGAAGCGGACCATCT
CTATCTGGGGCACCACCGATTACCCCCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 31)
SP_Ply_T65C_G293C_C428A_PlyD1_nIgK (5′ UTR, ORF, 3′ UTR)
TCAAGCTTTTGGACCCTCGTACAGAAGCTAATACGACTCACTATAGGGAAATAAGAGAGAAAAGAAGAGTAAGAAGAAATATAA
GAGCCACCATGGAAACCCCTGCCCAGCTGCTGTTCCTGCTGCTGCTGTGGCTGCCTGACACCACCGGCATGGCCAACAAGGCCG
TGAACGACTTCATCCTGGCCATGAACTACGACAAGAAGAAGCTGCTGACCCACCAGGGCGAGAGCATCGAGAACAGATTCATCA
AAGAGGGCAACCAGCTGCCCGACGAGTTCGTCGTGATCGAGCGGAAGAAGCGGAGCCTGAGCACCAACACCAGCGACATCAGCG
TGACCGCCTGCAACGACAGCAGACTGTATCCTGGCGCCCTGCTGGTGGTGGACGAGACACTGCTGGAAAACAACCCCACCCTGC
TGGCCGTGGACAGAGCCCCTATGACCTACAGCATCGACCTGCCTGGCCTGGCCAGCAGCGATAGCTTTCTGCAGGTGGAAGATC
CCAGCAACAGCAGCGTGCGGGGAGCCGTGAATGACCTGCTGGCTAAGTGGCACCAGGACTACGGCCAAGTGAACAACGTGCCCG
CCAGAATGCAGTACGAGAAGATCACCGCCCACTCCATGGAACAGCTGAAAGTGAAGTTCGGCAGCGACTTCGAGAAAACCGGCA
ACAGCCTGGACATCGACTTCAACAGCGTGCACAGCGGCGAGAAGCAGATCCAGATCGTGAACTTCAAGCAGATCTACTACACCG
TGTCCGTGGACGCCGTGAAGAACCCCGGGGACGTGTTCCAGGATACCGTGACCGTGGAAGATCTGAAGCAGCGGGGCATCAGCG
CCGAGAGGCCACTGGTGTACATCAGCTCTGTGGCCTACGGCAGACAGGTGTACCTGAAGCTGGAAACCACCTCCAAGAGCGACG
AGGTGGAAGCCGCCTTCGAGGCCCTGATCAAGGGCGTGAAAGTGGCCCCTCAGACCGAGTGGAAGCAGATTCTGGACAACACCG
AAGTGAAAGCCGTGATCCTGTGCGGCGACCCTTCTAGCGGAGCCAGAGTCGTGACAGGCAAGGTGGACATGGTGGAAGATCTGA
TCCAGGAAGGCAGCCGGTTCACCGCCGATCACCCTGGCCTGCCTATCAGCTACACCACAAGCTTTCTGAGAGACAACGTGGTGG
CCACATTCCAGAACAGCACCGACTACGTGGAAACAAAAGTGACAGCCTACCGGAACGGCGATCTGCTGCTGGATCACTCCGGCG
CCTACGTGGCCCAGTACTACATCACCTGGGACGAGCTGAGCTACGATCACCAGGGCAAAGAGGTGCTGACCCCCAAGGCCTGGG
ACAGAAACGGCCAGGATCTGACAGCCCACTTCACAACCAGCATCCCCCTGAAGGGCAACGTGCGGAACCTGAGCGTGAAGATCA
GAGAAGCCACCGGACTGGCCTGGGAGTGGTGGCGGACAGTGTACGAAAAGACCGACCTGCCCCTCGTGCGGAAGCGGACCATCT
CTATCTGGGGCACCACGCTGTATCCTCAGGTGGAAGATAAGGTGGAAAACGACTGATAATAGGCTGGAGCCTCGGTGGCCATGC
TTCTTGCCCCTTGGGCCTCCCCCCAGCCCCTCCTCCCCTTCCTGCACCCGTACCCCCGTGGTCTTTGAATAAAGTCTGAGTGGG
CGGC(SEQ ID NO: 32)
Sp_Ply_D205R_NGM_nIgK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCGACACCAGCGACATCAGCGTGACCGCC
ACCAACGACGCCAGACTGTATCCTGGCGCTCTGCTGGTGGTGGACGAGACACTGCTGGAAAACAACCCCATCCTGCTGGCCGTG
GACAGAGCCCCCATGACCTACAGCATCGACCTGCCTGGCCTGGCCAGCAGCGATAGCTTTCTGCAGGTGGAAGATCCCAGCAAC
AGCGCCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
CGGGCCGTGAAGAACCCTGGGGACGTGTTCCAGGATACCGTGACCGTGGAAGATCTGAAGCAGCGGGGCATCAGCGCCGAGAGG
CCACTGGTGTACATCAGCAGCGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGGGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACTCCGCCGACTACGTGGAAACAAAAGTGACCGCCTACCGGAACGGCGATCTGCTGCTGGATCACTCCGGCGCCTATGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAGTGC
ACCGGACTGGCCTGGGAGTGGTGGCGGACCGTGTACGAAAAGACCGACCTGCCCCTCGTGCGGAAGCGGACCATCTCTATCTGG
GGCACCACGCTGTATCCTCAGGTGGAAGATAAGGTGGAAAACGAC(SEQ ID NO: 56)
SP_Ply_L460D_NGM_nIgK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCGACACCAGCGACATCAGCGTGACCGCC
ACCAACGACGCCAGACTGTATCCTGGCGCTCTGCTGGTGGTGGACGAGACACTGCTGGAAAACAACCCCATCCTGCTGGCCGTG
GACAGAGCCCCCATGACCTACAGCATCGACCTGCCTGGCCTGGCCAGCAGCGATAGCTTTCTGCAGGTGGAAGATCCCAGCAAC
AGCGCCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
GACGCCGTGAAGAACCCCGGGGACGTGTTCCAGGATACCGTGACCGTGGAAGATCTGAAGCAGCGGGGCATCAGCGCCGAGAGG
CCACTGGTGTACATCAGCAGCGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGGGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACTCCGCCGACTACGTGGAAACAAAAGTGACCGCCTACCGGAACGGCGATCTGCTGCTGGATCACTCCGGCGCCTATGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAGTGC
ACCGGACTGGCCTGGGAGTGGTGGCGGACCGTGTACGAAAAGACCGACCTGCCCCTCGTGCGGAAGCGGACCATCTCTATCTGG
GGCACCACCGACTACCCCCAGGTGGAAGATAAGGTGGAAAACGAC(SEQ ID NO: 57)
SP_Ply_T65C_G293C_C428A_PlyD1_ NGM_nIgK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCGACACCAGCGACATCAGCGTGACCGCC
TGCAACGACGCCAGACTGTATCCTGGCGCTCTGCTGGTGGTGGACGAGACACTGCTGGAAAACAACCCCATCCTGCTGGCCGTG
GACAGAGCCCCCATGACCTACAGCATCGACCTGCCTGGCCTGGCCAGCAGCGATAGCTTTCTGCAGGTGGAAGATCCCAGCAAC
AGCGCCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
GACGCCGTGAAGAACCCCGGGGACGTGTTCCAGGATACCGTGACCGTGGAAGATCTGAAGCAGCGGGGCATCAGCGCCGAGAGG
CCACTGGTGTACATCAGCAGCGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGTGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACTCCGCCGACTACGTGGAAACAAAAGTGACAGCCTACCGGAACGGCGATCTGCTGCTGGATCACTCCGGCGCCTATGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAAGCC
ACCGGACTGGCCTGGGAGTGGTGGCGGACAGTGTACGAAAAGACCGACCTGCCCCTCGTGCGGAAGCGGACCATCTCTATCTGG
GGCACCACGCTGTATCCTCAGGTGGAAGATAAGGTGGAAAACGAC(SEQ ID NO: 58)
SP_Ply_D205R_nIgK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCAACACCAGCGACATCAGCGTGACCGCC
ACCAACGACAGCAGACTGTATCCTGGCGCCCTGCTGGTGGTGGACGAGACACTGCTGGAAAACAACCCCACCCTGCTGGCCGTG
GACAGAGCCCCTATGACCTACAGCATCGACCTGCCTGGCCTGGCCAGCAGCGATAGCTTTCTGCAGGTGGAAGATCCCAGCAAC
AGCAGCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
CGGGCCGTGAAGAACCCTGGGGACGTGTTCCAGGATACCGTGACCGTGGAAGATCTGAAGCAGCGGGGCATCAGCGCCGAGAGG
CCACTGGTGTACATCAGCTCTGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGGGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACAGCACCGACTACGTGGAAACAAAAGTGACCGCCTACCGGAACGGCGATCTGCTGCTGGATCACTCCGGCGCCTACGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAGTGC
ACCGGACTGGCCTGGGAGTGGTGGCGGACCGTGTACGAAAAGACCGACCTGCCCCTCGTGCGGAAGCGGACCATCTCTATCTGG
GGCACCACGCTGTATCCTCAGGTGGAAGATAAGGTGGAAAACGAC(SEQ ID NO: 59)
SP_Ply_L460D_nigK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCAACACCAGCGACATCAGCGTGACCGCC
ACCAACGACAGCAGACTGTATCCTGGCGCCCTGCTGGTGGTGGACGAGACACTGCTGGAAAACAACCCCACCCTGCTGGCCGTG
GACAGAGCCCCTATGACCTACAGCATCGACCTGCCTGGCCTGGCCAGCAGCGATAGCTTTCTGCAGGTGGAAGATCCCAGCAAC
AGCAGCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
GACGCCGTGAAGAACCCCGGGGACGTGTTCCAGGATACCGTGACCGTGGAAGATCTGAAGCAGCGGGGCATCAGCGCCGAGAGG
CCACTGGTGTACATCAGCTCTGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGGGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACAGCACCGACTACGTGGAAACAAAAGTGACCGCCTACCGGAACGGCGATCTGCTGCTGGATCACTCCGGCGCCTACGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAGTGC
ACCGGACTGGCCTGGGAGTGGTGGCGGACCGTGTACGAAAAGACCGACCTGCCCCTCGTGCGGAAGCGGACCATCTCTATCTGG
GGCACCACCGATTACCCCCAGGTGGAAGATAAGGTGGAAAACGAC(SEQ ID NO: 60)
SP_Ply_T65C_G293C_C428A_PlyD1_nIgK (ORF)
ATGGAAACCCCTGCCCAGCTGCTGTTCCTGCTGCTGCTGTGGCTGCCTGACACCACCGGCATGGCCAACAAGGCCGTGAACGAC
TTCATCCTGGCCATGAACTACGACAAGAAGAAGCTGCTGACCCACCAGGGCGAGAGCATCGAGAACAGATTCATCAAAGAGGGC
AACCAGCTGCCCGACGAGTTCGTCGTGATCGAGCGGAAGAAGCGGAGCCTGAGCACCAACACCAGCGACATCAGCGTGACCGCC
TGCAACGACAGCAGACTGTATCCTGGCGCCCTGCTGGTGGTGGACGAGACACTGCTGGAAAACAACCCCACCCTGCTGGCCGTG
GACAGAGCCCCTATGACCTACAGCATCGACCTGCCTGGCCTGGCCAGCAGCGATAGCTTTCTGCAGGTGGAAGATCCCAGCAAC
AGCAGCGTGCGGGGAGCCGTGAATGACCTGCTGGCTAAGTGGCACCAGGACTACGGCCAAGTGAACAACGTGCCCGCCAGAATG
CAGTACGAGAAGATCACCGCCCACTCCATGGAACAGCTGAAAGTGAAGTTCGGCAGCGACTTCGAGAAAACCGGCAACAGCCTG
GACATCGACTTCAACAGCGTGCACAGCGGCGAGAAGCAGATCCAGATCGTGAACTTCAAGCAGATCTACTACACCGTGTCCGTG
GACGCCGTGAAGAACCCCGGGGACGTGTTCCAGGATACCGTGACCGTGGAAGATCTGAAGCAGCGGGGCATCAGCGCCGAGAGG
CCACTGGTGTACATCAGCTCTGTGGCCTACGGCAGACAGGTGTACCTGAAGCTGGAAACCACCTCCAAGAGCGACGAGGTGGAA
GCCGCCTTCGAGGCCCTGATCAAGGGCGTGAAAGTGGCCCCTCAGACCGAGTGGAAGCAGATTCTGGACAACACCGAAGTGAAA
GCCGTGATCCTGTGCGGCGACCCTTCTAGCGGAGCCAGAGTCGTGACAGGCAAGGTGGACATGGTGGAAGATCTGATCCAGGAA
GGCAGCCGGTTCACCGCCGATCACCCTGGCCTGCCTATCAGCTACACCACAAGCTTTCTGAGAGACAACGTGGTGGCCACATTC
CAGAACAGCACCGACTACGTGGAAACAAAAGTGACAGCCTACCGGAACGGCGATCTGCTGCTGGATCACTCCGGCGCCTACGTG
GCCCAGTACTACATCACCTGGGACGAGCTGAGCTACGATCACCAGGGCAAAGAGGTGCTGACCCCCAAGGCCTGGGACAGAAAC
GGCCAGGATCTGACAGCCCACTTCACAACCAGCATCCCCCTGAAGGGCAACGTGCGGAACCTGAGCGTGAAGATCAGAGAAGCC
ACCGGACTGGCCTGGGAGTGGTGGCGGACAGTGTACGAAAAGACCGACCTGCCCCTCGTGCGGAAGCGGACCATCTCTATCTGG
GGCACCACGCTGTATCCTCAGGTGGAAGATAAGGTGGAAAACGAC(SEQ ID NO: 61)
mRNA Sequences
S. pneumoniae pneumolysin gene, complete cds; Accession No. M17717
UUACAAGACCAACCUUGAUUGACUUAGAUAAGGUAUUUAUGUUGGAUAAUACGGUUAUUCCGACUUCUUAUCUAGCCAGACGGC
GACGCAAUGUCUCAGAAGAAUUGUACGAGGAAAUUUUGGAUCACUUAGUCCAACCACGGCUGAUUUCGCUGAACAAGUCUGAGU
UUAUGCAACUCAAUCCAGGAACUUAUUAGGAGGAGAAGAUGGCAAAUAAAGCAGUAAAUGACUUUAUACUAGCUAUGAAUUACG
AUAAAAAGAAACUCUUGACCCAUCAGGGAGAAAGUAUUGAAAAUCGUUUCAUCAAAGAGGGUAAUCAGCUACCCGAUGAGUUUG
UUGUUAUCGAAAGAAAGAAGCGGAGCUUGUCGACAAAUACAAGUGAUAUUUCUGUAACAGCUACCAACGACAGUCGCCUCUAUC
CUGGAGCACUUCUCGUAGUGGAUGAGACCUUGUUAGAGAAUAAUCCCACUCUUCUUGCGGUUGAUCGUGCUCCGAUGACUUAUA
GUAUUGAUUUGCCUGGUUUGGCAAGUAGCGAUAGCUUUCUCCAAGUGGAAGACCCCAGCAAUUCAAGUGUUCGCGGAGCGGUAA
ACGAUUUGUUGGCUAAGUGGCAUCAAGAUUAUGGUCAGGUCAAUAAUGUCCCAGCUAGAAUGCAGUAUGAAAAAAUAACGGCUC
ACAGCAUGGAACAACUCAAGGUCAAGUUUGGUUCUGACUUUGAAAAGACAGGGAAUUCUCUUGAUAUUGAUUUUAACUCUGUCC
AUUCAGGUGAAAAGCAGAUUCAGAUUGUUAAUUUUAAGCAGAUUUAUUAUACAGUCAGCGUAGACGCUGUUAAAAAUCCAGGAG
AUGUGUUUCAAGAUACUGUAACGGUAGAGGAUUUAAAACAGAGAGGAAUUUCUGCAGAGCGUCCUUUGGUCUAUAUUUCGAGUG
UUGCUUAUGGGCGCCAAGUCUAUCUCAAGUUGGAAACCACGAGUAAGAGUGAUGAAGUAGAGGCUGCUUUUGAAGCUUUGAUAA
AAGGAGUCAAGGUAGCUCCUCAGACAGAGUGGAAGCAGAUUUUGGACAAUACAGAAGUGAAGGCGGUUAUUUUAGGGGGCGACC
CAAGUUCGGGUGCCCGAGUUGUAACAGGCAAGGUGGAUAUGGUAGAGGACUUGAUUCAAGAAGGCAGUCGCUUUACAGCAGAUC
AUCCAGGCUUGCCGAUUUCCUAUACAACUUCUUUUUUACGUGACAAUGUAGUUGCGACCUUUCAAAACAGUACAGACUAUGUUG
AGACUAAGGUUACAGCUUACAGAAACGGAGAUUUACUGCUGGAUCAUAGUGGUGCCUAUGUUGCCCAAUAUUAUAUUACUUGGG
AUGAAUUAUCCUAUGAUCAUCAAGGUAAGGAAGUCUUGACUCCUAAGGCUUGGGACAGAAAUGGGCAGGAUUUGACGGCUCACU
UUACCACUAGUAUUCCUUUAAAAGGGAAUGUUCGUAAUCUCUCUGUCAAAAUUAGAGAGUGUACCGGGCUUGCCUGGGAAUGGU
GGCGUACGGUUUAUGAAAAAACCGAUUUGCCACUAGUGCGUAAGCGGACGAUUUCUAUUUGGGGAACAACUCUCUAUCCUCAGG
UAGAGGAUAAGGUAGAAAAUGACUAGGAGAGGAGAAUGCUUGCGACAAAAAGA (SEQ ID NO: 5)
Sp_Ply_D205R_NGM_nIgK (5′UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCGACACCAGCGACAUCAGCG
UGACCGCCACCAACGACGCCAGACUGUAUCCUGGCGCUCUGCUGGUGGUGGACGAGACACUGCUGGAAAACAACCCCAUCCUGC
UGGCCGUGGACAGAGCCCCCAUGACCUACAGCAUCGACCUGCCUGGCCUGGCCAGCAGCGAUAGCUUUCUGCAGGUGGAAGAUC
CCAGCAACAGCGCCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGCGGGCCGUGAAGAACCCUGGGGACGUGUUCCAGGAUACCGUGACCGUGGAAGAUCUGAAGCAGCGGGGCAUCAGCG
CCGAGAGGCCACUGGUGUACAUCAGCAGCGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGGGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACUCCGCCGACUACGUGGAAACAAAAGUGACCGCCUACCGGAACGGCGAUCUGCUGCUGGAUCACUCCGGCG
CCUAUGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAGUGCACCGGACUGGCCUGGGAGUGGUGGCGGACCGUGUACGAAAAGACCGACCUGCCCCUCGUGCGGAAGCGGACCAUCU
CUAUCUGGGGCACCACGCUGUAUCCUCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGGG
CGGC(SEQ ID NO: 6)
SP_Ply_L460D_NGM_nIgK (5′ UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCGACACCAGCGACAUCAGCG
UGACCGCCACCAACGACGCCAGACUGUAUCCUGGCGCUCUGCUGGUGGUGGACGAGACACUGCUGGAAAACAACCCCAUCCUGC
UGGCCGUGGACAGAGCCCCCAUGACCUACAGCAUCGACCUGCCUGGCCUGGCCAGCAGCGAUAGCUUUCUGCAGGUGGAAGAUC
CCAGCAACAGCGCCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGGACGCCGUGAAGAACCCCGGGGACGUGUUCCAGGAUACCGUGACCGUGGAAGAUCUGAAGCAGCGGGGCAUCAGCG
CCGAGAGGCCACUGGUGUACAUCAGCAGCGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGGGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACUCCGCCGACUACGUGGAAACAAAAGUGACCGCCUACCGGAACGGCGAUCUGCUGCUGGAUCACUCCGGCG
CCUAUGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAGUGCACCGGACUGGCCUGGGAGUGGUGGCGGACCGUGUACGAAAAGACCGACCUGCCCCUCGUGCGGAAGCGGACCAUCU
CUAUCUGGGGCACCACCGACUACCCCCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGG
GCGGC(SEQ ID NO: 7)
SP_Ply_T65C_G293C_C428A_PlyD1_ NGM_nIgK (5′ UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCGACACCAGCGACAUCAGCG
UGACCGCCUGCAACGACGCCAGACUGUAUCCUGGCGCUCUGCUGGUGGUGGACGAGACACUGCUGGAAAACAACCCCAUCCUGC
UGGCCGUGGACAGAGCCCCCAUGACCUACAGCAUCGACCUGCCUGGCCUGGCCAGCAGCGAUAGCUUUCUGCAGGUGGAAGAUC
CCAGCAACAGCGCCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGGACGCCGUGAAGAACCCCGGGGACGUGUUCCAGGAUACCGUGACCGUGGAAGAUCUGAAGCAGCGGGGCAUCAGCG
CCGAGAGGCCACUGGUGUACAUCAGCAGCGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGUGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACUCCGCCGACUACGUGGAAACAAAAGUGACAGCCUACCGGAACGGCGAUCUGCUGCUGGAUCACUCCGGCG
CCUAUGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAAGCCACCGGACUGGCCUGGGAGUGGUGGCGGACAGUGUACGAAAAGACCGACCUGCCCCUCGUGCGGAAGCGGACCAUCU
CUAUCUGGGGCACCACGCUGUAUCCUCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGGG
CGGC(SEQ ID NO: 8)
SP_Ply_D205R_nIgK (5′ UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCAACACCAGCGACAUCAGCG
UGACCGCCACCAACGACAGCAGACUGUAUCCUGGCGCCCUGCUGGUGGUGGACGAGACACUGCUGGAAAACAACCCCACCCUGC
UGGCCGUGGACAGAGCCCCUAUGACCUACAGCAUCGACCUGCCUGGCCUGGCCAGCAGCGAUAGCUUUCUGCAGGUGGAAGAUC
CCAGCAACAGCAGCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGCGGGCCGUGAAGAACCCUGGGGACGUGUUCCAGGAUACCGUGACCGUGGAAGAUCUGAAGCAGCGGGGCAUCAGCG
CCGAGAGGCCACUGGUGUACAUCAGCUCUGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGGGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACAGCACCGACUACGUGGAAACAAAAGUGACCGCCUACCGGAACGGCGAUCUGCUGCUGGAUCACUCCGGCG
CCUACGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAGUGCACCGGACUGGCCUGGGAGUGGUGGCGGACCGUGUACGAAAAGACCGACCUGCCCCUCGUGCGGAAGCGGACCAUCU
CUAUCUGGGGCACCACGCUGUAUCCUCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGGG
CGGC(SEQ ID NO: 33)
SP_Ply_L460D_nIgK (5′ UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCAACACCAGCGACAUCAGCG
UGACCGCCACCAACGACAGCAGACUGUAUCCUGGCGCCCUGCUGGUGGUGGACGAGACACUGCUGGAAAACAACCCCACCCUGC
UGGCCGUGGACAGAGCCCCUAUGACCUACAGCAUCGACCUGCCUGGCCUGGCCAGCAGCGAUAGCUUUCUGCAGGUGGAAGAUC
CCAGCAACAGCAGCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGGACGCCGUGAAGAACCCCGGGGACGUGUUCCAGGAUACCGUGACCGUGGAAGAUCUGAAGCAGCGGGGCAUCAGCG
CCGAGAGGCCACUGGUGUACAUCAGCUCUGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGGGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACAGCACCGACUACGUGGAAACAAAAGUGACCGCCUACCGGAACGGCGAUCUGCUGCUGGAUCACUCCGGCG
CCUACGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAGUGCACCGGACUGGCCUGGGAGUGGUGGCGGACCGUGUACGAAAAGACCGACCUGCCCCUCGUGCGGAAGCGGACCAUCU
CUAUCUGGGGCACCACCGAUUACCCCCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGGG
CGGC(SEQ ID NO: 34)
SP_Ply_T65C_G293C_C428A_PlyD1_nIgK (5′ UTR, ORF, 3′ UTR)
UCAAGCUUUUGGACCCUCGUACAGAAGCUAAUACGACUCACUAUAGGGAAAUAAGAGAGAAAAGAAGAGUAAGAAGAAAUAUAA
GAGCCACCAUGGAAACCCCUGCCCAGCUGCUGUUCCUGCUGCUGCUGUGGCUGCCUGACACCACCGGCAUGGCCAACAAGGCCG
UGAACGACUUCAUCCUGGCCAUGAACUACGACAAGAAGAAGCUGCUGACCCACCAGGGCGAGAGCAUCGAGAACAGAUUCAUCA
AAGAGGGCAACCAGCUGCCCGACGAGUUCGUCGUGAUCGAGCGGAAGAAGCGGAGCCUGAGCACCAACACCAGCGACAUCAGCG
UGACCGCCUGCAACGACAGCAGACUGUAUCCUGGCGCCCUGCUGGUGGUGGACGAGACACUGCUGGAAAACAACCCCACCCUGC
UGGCCGUGGACAGAGCCCCUAUGACCUACAGCAUCGACCUGCCUGGCCUGGCCAGCAGCGAUAGCUUUCUGCAGGUGGAAGAUC
CCAGCAACAGCAGCGUGCGGGGAGCCGUGAAUGACCUGCUGGCUAAGUGGCACCAGGACUACGGCCAAGUGAACAACGUGCCCG
CCAGAAUGCAGUACGAGAAGAUCACCGCCCACUCCAUGGAACAGCUGAAAGUGAAGUUCGGCAGCGACUUCGAGAAAACCGGCA
ACAGCCUGGACAUCGACUUCAACAGCGUGCACAGCGGCGAGAAGCAGAUCCAGAUCGUGAACUUCAAGCAGAUCUACUACACCG
UGUCCGUGGACGCCGUGAAGAACCCCGGGGACGUGUUCCAGGAUACCGUGACCGUGGAAGAUCUGAAGCAGCGGGGCAUCAGCG
CCGAGAGGCCACUGGUGUACAUCAGCUCUGUGGCCUACGGCAGACAGGUGUACCUGAAGCUGGAAACCACCUCCAAGAGCGACG
AGGUGGAAGCCGCCUUCGAGGCCCUGAUCAAGGGCGUGAAAGUGGCCCCUCAGACCGAGUGGAAGCAGAUUCUGGACAACACCG
AAGUGAAAGCCGUGAUCCUGUGCGGCGACCCUUCUAGCGGAGCCAGAGUCGUGACAGGCAAGGUGGACAUGGUGGAAGAUCUGA
UCCAGGAAGGCAGCCGGUUCACCGCCGAUCACCCUGGCCUGCCUAUCAGCUACACCACAAGCUUUCUGAGAGACAACGUGGUGG
CCACAUUCCAGAACAGCACCGACUACGUGGAAACAAAAGUGACAGCCUACCGGAACGGCGAUCUGCUGCUGGAUCACUCCGGCG
CCUACGUGGCCCAGUACUACAUCACCUGGGACGAGCUGAGCUACGAUCACCAGGGCAAAGAGGUGCUGACCCCCAAGGCCUGGG
ACAGAAACGGCCAGGAUCUGACAGCCCACUUCACAACCAGCAUCCCCCUGAAGGGCAACGUGCGGAACCUGAGCGUGAAGAUCA
GAGAAGCCACCGGACUGGCCUGGGAGUGGUGGCGGACAGUGUACGAAAAGACCGACCUGCCCCUCGUGCGGAAGCGGACCAUCU
CUAUCUGGGGCACCACGCUGUAUCCUCAGGUGGAAGAUAAGGUGGAAAACGACUGAUAAUAGGCUGGAGCCUCGGUGGCCAUGC
UUCUUGCCCCUUGGGCCUCCCCCCAGCCCCUCCUCCCCUUCCUGCACCCGUACCCCCGUGGUCUUUGAAUAAAGUCUGAGUGGG
CGGC(SEQ ID NO: 35)
Sp_Ply_D205R_NGM_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCGACACCAGCGACAUCAGCGUGACCGCC
ACCAACGACGCCAGACUGUAUCCUGGCGCUCUGCUGGUGGUGGACGAGACACUGCUGGAAAACAACCCCAUCCUGCUGGCCGUG
GACAGAGCCCCCAUGACCUACAGCAUCGACCUGCCUGGCCUGGCCAGCAGCGAUAGCUUUCUGCAGGUGGAAGAUCCCAGCAAC
AGCGCCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
CGGGCCGUGAAGAACCCUGGGGACGUGUUCCAGGAUACCGUGACCGUGGAAGAUCUGAAGCAGCGGGGCAUCAGCGCCGAGAGG
CCACUGGUGUACAUCAGCAGCGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGGGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACUCCGCCGACUACGUGGAAACAAAAGUGACCGCCUACCGGAACGGCGAUCUGCUGCUGGAUCACUCCGGCGCCUAUGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAGUGC
ACCGGACUGGCCUGGGAGUGGUGGCGGACCGUGUACGAAAAGACCGACCUGCCCCUCGUGCGGAAGCGGACCAUCUCUAUCUGG
GGCACCACGCUGUAUCCUCAGGUGGAAGAUAAGGUGGAAAACGAC(SEQ ID NO: 62)
SP_Ply_L460D_NGM_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCGACACCAGCGACAUCAGCGUGACCGCC
ACCAACGACGCCAGACUGUAUCCUGGCGCUCUGCUGGUGGUGGACGAGACACUGCUGGAAAACAACCCCAUCCUGCUGGCCGUG
GACAGAGCCCCCAUGACCUACAGCAUCGACCUGCCUGGCCUGGCCAGCAGCGAUAGCUUUCUGCAGGUGGAAGAUCCCAGCAAC
AGCGCCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
GACGCCGUGAAGAACCCCGGGGACGUGUUCCAGGAUACCGUGACCGUGGAAGAUCUGAAGCAGCGGGGCAUCAGCGCCGAGAGG
CCACUGGUGUACAUCAGCAGCGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGGGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACUCCGCCGACUACGUGGAAACAAAAGUGACCGCCUACCGGAACGGCGAUCUGCUGCUGGAUCACUCCGGCGCCUAUGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAGUGC
ACCGGACUGGCCUGGGAGUGGUGGCGGACCGUGUACGAAAAGACCGACCUGCCCCUCGUGCGGAAGCGGACCAUCUCUAUCUGG
GGCACCACCGACUACCCCCAGGUGGAAGAUAAGGUGGAAAACGAC(SEQ ID NO: 63)
SP_Ply_T65C_G293C_C428A_PlyD1_ NGM_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCGACACCAGCGACAUCAGCGUGACCGCC
UGCAACGACGCCAGACUGUAUCCUGGCGCUCUGCUGGUGGUGGACGAGACACUGCUGGAAAACAACCCCAUCCUGCUGGCCGUG
GACAGAGCCCCCAUGACCUACAGCAUCGACCUGCCUGGCCUGGCCAGCAGCGAUAGCUUUCUGCAGGUGGAAGAUCCCAGCAAC
AGCGCCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
GACGCCGUGAAGAACCCCGGGGACGUGUUCCAGGAUACCGUGACCGUGGAAGAUCUGAAGCAGCGGGGCAUCAGCGCCGAGAGG
CCACUGGUGUACAUCAGCAGCGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGUGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACUCCGCCGACUACGUGGAAACAAAAGUGACAGCCUACCGGAACGGCGAUCUGCUGCUGGAUCACUCCGGCGCCUAUGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAAGCC
ACCGGACUGGCCUGGGAGUGGUGGCGGACAGUGUACGAAAAGACCGACCUGCCCCUCGUGCGGAAGCGGACCAUCUCUAUCUGG
GGCACCACGCUGUAUCCUCAGGUGGAAGAUAAGGUGGAAAACGAC(SEQ ID NO: 64)
SP_Ply_D205R_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCAACACCAGCGACAUCAGCGUGACCGCC
ACCAACGACAGCAGACUGUAUCCUGGCGCCCUGCUGGUGGUGGACGAGACACUGCUGGAAAACAACCCCACCCUGCUGGCCGUG
GACAGAGCCCCUAUGACCUACAGCAUCGACCUGCCUGGCCUGGCCAGCAGCGAUAGCUUUCUGCAGGUGGAAGAUCCCAGCAAC
AGCAGCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
CGGGCCGUGAAGAACCCUGGGGACGUGUUCCAGGAUACCGUGACCGUGGAAGAUCUGAAGCAGCGGGGCAUCAGCGCCGAGAGG
CCACUGGUGUACAUCAGCUCUGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGGGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACAGCACCGACUACGUGGAAACAAAAGUGACCGCCUACCGGAACGGCGAUCUGCUGCUGGAUCACUCCGGCGCCUACGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAGUGC
ACCGGACUGGCCUGGGAGUGGUGGCGGACCGUGUACGAAAAGACCGACCUGCCCCUCGUGCGGAAGCGGACCAUCUCUAUCUGG
GGCACCACGCUGUAUCCUCAGGUGGAAGAUAAGGUGGAAAACGAC(SEQ ID NO: 65)
SP_Ply_L460D_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCAACACCAGCGACAUCAGCGUGACCGCC
ACCAACGACAGCAGACUGUAUCCUGGCGCCCUGCUGGUGGUGGACGAGACACUGCUGGAAAACAACCCCACCCUGCUGGCCGUG
GACAGAGCCCCUAUGACCUACAGCAUCGACCUGCCUGGCCUGGCCAGCAGCGAUAGCUUUCUGCAGGUGGAAGAUCCCAGCAAC
AGCAGCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
GACGCCGUGAAGAACCCCGGGGACGUGUUCCAGGAUACCGUGACCGUGGAAGAUCUGAAGCAGCGGGGCAUCAGCGCCGAGAGG
CCACUGGUGUACAUCAGCUCUGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGGGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACAGCACCGACUACGUGGAAACAAAAGUGACCGCCUACCGGAACGGCGAUCUGCUGCUGGAUCACUCCGGCGCCUACGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAGUGC
ACCGGACUGGCCUGGGAGUGGUGGCGGACCGUGUACGAAAAGACCGACCUGCCCCUCGUGCGGAAGCGGACCAUCUCUAUCUGG
GGCACCACCGAUUACCCCCAGGUGGAAGAUAAGGUGGAAAACGAC(SEQ ID NO: 66)
SP_Ply_T65C_G293C_C428A_PlyD1_nIgK (ORF)
AUGGAAACCCCUGCCCAGCUGCUGUUCCUGCUGCUGCUGUGGCUGCCUGACACCACCGGCAUGGCCAACAAGGCCGUGAACGAC
UUCAUCCUGGCCAUGAACUACGACAAGAAGAAGCUGCUGACCCACCAGGGCGAGAGCAUCGAGAACAGAUUCAUCAAAGAGGGC
AACCAGCUGCCCGACGAGUUCGUCGUGAUCGAGCGGAAGAAGCGGAGCCUGAGCACCAACACCAGCGACAUCAGCGUGACCGCC
UGCAACGACAGCAGACUGUAUCCUGGCGCCCUGCUGGUGGUGGACGAGACACUGCUGGAAAACAACCCCACCCUGCUGGCCGUG
GACAGAGCCCCUAUGACCUACAGCAUCGACCUGCCUGGCCUGGCCAGCAGCGAUAGCUUUCUGCAGGUGGAAGAUCCCAGCAAC
AGCAGCGUGCGGGGAGCCGUGAAUGACCUGCUGGCUAAGUGGCACCAGGACUACGGCCAAGUGAACAACGUGCCCGCCAGAAUG
CAGUACGAGAAGAUCACCGCCCACUCCAUGGAACAGCUGAAAGUGAAGUUCGGCAGCGACUUCGAGAAAACCGGCAACAGCCUG
GACAUCGACUUCAACAGCGUGCACAGCGGCGAGAAGCAGAUCCAGAUCGUGAACUUCAAGCAGAUCUACUACACCGUGUCCGUG
GACGCCGUGAAGAACCCCGGGGACGUGUUCCAGGAUACCGUGACCGUGGAAGAUCUGAAGCAGCGGGGCAUCAGCGCCGAGAGG
CCACUGGUGUACAUCAGCUCUGUGGCCUACGGCAGACAGGUGUACCUGAAGCUGGAAACCACCUCCAAGAGCGACGAGGUGGAA
GCCGCCUUCGAGGCCCUGAUCAAGGGCGUGAAAGUGGCCCCUCAGACCGAGUGGAAGCAGAUUCUGGACAACACCGAAGUGAAA
GCCGUGAUCCUGUGCGGCGACCCUUCUAGCGGAGCCAGAGUCGUGACAGGCAAGGUGGACAUGGUGGAAGAUCUGAUCCAGGAA
GGCAGCCGGUUCACCGCCGAUCACCCUGGCCUGCCUAUCAGCUACACCACAAGCUUUCUGAGAGACAACGUGGUGGCCACAUUC
CAGAACAGCACCGACUACGUGGAAACAAAAGUGACAGCCUACCGGAACGGCGAUCUGCUGCUGGAUCACUCCGGCGCCUACGUG
GCCCAGUACUACAUCACCUGGGACGAGCUGAGCUACGAUCACCAGGGCAAAGAGGUGCUGACCCCCAAGGCCUGGGACAGAAAC
GGCCAGGAUCUGACAGCCCACUUCACAACCAGCAUCCCCCUGAAGGGCAACGUGCGGAACCUGAGCGUGAAGAUCAGAGAAGCC
ACCGGACUGGCCUGGGAGUGGUGGCGGACAGUGUACGAAAAGACCGACCUGCCCCUCGUGCGGAAGCGGACCAUCUCUAUCUGG
GGCACCACGCUGUAUCCUCAGGUGGAAGAUAAGGUGGAAAACGAC(SEQ ID NO: 67)

TABLE 2
Pneumolysin Amino Acid Sequences
Descripti Sequence SEQ ID NO:

pneumolysin [Streptococcus pneumoniae]; Accession No. AJS15225.1
MANKAVNDFILAMNYDKKKLLTHQGESIENRFIKEGNQLPDEFVVIERKKRSLSTNTSDISVTATNDSRLYPGALLVVDETLL
ENNPTLLAVDRAPMTYSIDLPGLASSDSFLQVEDPSNSSVRGAVNDLLAKWHQDYGQVNNVPARMQYEKITAHSMEQLKVKFG
SDFEKTGNSLDIDFNSVHSGEKQIQIVNFKQIYYTVSVDAVKNPGDVFQDTVTVEDLKQRGISAERPLVYISSVAYGRQVYLK
LETTSKSDEVEAAFEALIKGVKVAPQTEWKQILDNTEVKAVILGGDPSSGARVVTGKVDMVEDLIQEGSRFTADHPGLPISYT
TSFLRDNVVATFQNSTDYVETKVTAYRNGDLLLDHSGAYVAQYYITWNELSYDHQGKEVLTPKAWDRNGQDLTAHFTTSIPLK
GNVRNLSVKIRECTGLAWEWWRTVYEKTDLPLVRKRTISIWGTTLYPQVEDKVEND (SEQ ID NO: 9)
4ZGH: A|PDBID|CHAIN|SEQUENCE
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 10)
VAR1_D102F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIFLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 11)
VAR2_D102M
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIMLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 12)
VAR3_A107M
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLMSSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 13)
VAR4_A107Q
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLQSSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 14)
VARS_N120F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSFSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 15)
VAR6_E187R
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGRKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 16)
VAR7_E187L
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGLKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 17)
VAR8_D102F_A107Q
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIFLPGLQSSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 18)
VAR9_D102F_N120F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIFLPGLASSDSFLQVEDPSFSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 19)
VAR10_A107Q_N120F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLQSSDSFLQVEDPSFSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 20)
VAR11_D102F_A107Q_N120F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIFLPGLQSSDSFLQVEDPSFSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 21)
VAR12_D102F_A107Q_N120F_E187L
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIFLPGLQSSDSFLQVEDPSFSSVRGAVNDLLAKWHQDYGQVNNVPARMQYEKITAHSME
QLKVKFGSDFEKTGNSLDIDFNSVHSGLKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 22)
VAR13_D205F
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVFAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 23)
VAR14_D205P
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVPAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 24)
VAR15_G2931
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSNSSVRGAVNDLLAKWHQDYGQVNNVPARMQYEKITAHSME
QLKVKFGSDFEKTGNSLDIDFNSVHSGEKQIQIVNFKQIYYTVSVDAVRNPGDVFQDTVTVEDLKQRGISAERPLVYISSVAY
GRQVYLKLETTSKSDEVEAAFEALIKGVKVAPQTEWKQILDNTEVKAVILIGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 25)
VAR16_N120F_D205P
HHHHHHGSANKAVNDFILAMNYDKKKLLTHQGESIENRFIKEGNQLPDEFVVIERKKRSLSTSTSDISVTATNDSRLYPGALL
VVDETLLENNPTLLAVDRAPMTYSIDLPGLASSDSFLQVEDPSFSSVRGAVNDLLAKWHQDYGQVNNVPARMQYEKITAHSME
QLKVKFGSDFEKTGNSLDIDFNSVHSGEKQIQIVNFKQIYYTVSVPAVRNPGDVFQDTVTVEDLKQRGISAERPLVYISSVAY
GRQVYLKLETTSKSDEVEAAFEALIKGVKVAPQTEWKQILDNTEVKAVILGGDPSSGARVVTGKVDMVDDLIQEGSRFTADHP
GLPISYTTSFLRDNVVATFQNSTDYVETKVTAYRNGDLLLDHSGAYVAQYYITWDELSYDHQGKEVLTPKAWDRNGQDLTAHF
TTSIPLKGNVRNLSVKIRECTGLAWEWWRTVYEKTDLPLVRKRTISIWGTTLYPQVEDKVEN (SEQ ID NO: 26)
SP_Ply_D205R_NGM_nIgK
METPAQLLFLLLLWLPDTTGMANKAVNDFILAMNYDKKKLLTHQGESIENRFIKEGNQLPDEFVVIERKKRSLSTDTSDISVT
ATNDARLYPGALLVVDETLLENNPILLAVDRAPMTYSIDLPGLASSDSFLQVEDPSNSAVRGAVNDLLAKWHQDYGQVNNVPA
RMQYEKITAHSMEQLKVKFGSDFEKTGNSLDIDFNSVHSGEKQIQIVNFKQIYYTVSVRAVKNPGDVFQDTVTVEDLKQRGIS
AERPLVYISSVAYGRQVYLKLETTSKSDEVEAAFEALIKGVKVAPQTEWKQILDNTEVKAVILGGDPSSGARVVTGKVDMVED
LIQEGSRFTADHPGLPISYTTSFLRDNVVATFQNSADYVETKVTAYRNGDLLLDHSGAYVAQYYITWDELSYDHQGKEVLTPK
AWDRNGQDLTAHFTTSIPLKGNVRNLSVKIRECTGLAWEWWRTVYEKTDLPLVRKRTISIWGTTLYPQVEDKVEND
(SEQ ID NO: 27)
SP_Ply_L460D_NGM_nIgK
METPAQLLFLLLLWLPDTTGMANKAVNDFILAMNYDKKKLLTHQGESIENRFIKEGNQLPDEFVVIERKKRSLSTDTSDISVT
ATNDARLYPGALLVVDETLLENNPILLAVDRAPMTYSIDLPGLASSDSFLQVEDPSNSAVRGAVNDLLAKWHQDYGQVNNVPA
RMQYEKITAHSMEQLKVKFGSDFEKTGNSLDIDFNSVHSGEKQIQIVNFKQIYYTVSVDAVKNPGDVFQDTVTVEDLKQRGIS
AERPLVYISSVAYGRQVYLKLETTSKSDEVEAAFEALIKGVKVAPQTEWKQILDNTEVKAVILGGDPSSGARVVTGKVDMVED
LIQEGSRFTADHPGLPISYTTSFLRDNVVATFQNSADYVETKVTAYRNGDLLLDHSGAYVAQYYITWDELSYDHQGKEVLTPK
AWDRNGQDLTAHFTTSIPLKGNVRNLSVKIRECTGLAWEWWRTVYEKTDLPLVRKRTISIWGTTDYPQVEDKVEND
(SEQ ID NO: 28)
SP_Ply_T65C_G293C_C428A_PlyD1_NGM_nIgK
METPAQLLFLLLLWLPDTTGMANKAVNDFILAMNYDKKKLLTHQGESIENRFIKEGNQLPDEFVVIERKKRSLSTDTSDISVT
ACNDARLYPGALLVVDETLLENNPILLAVDRAPMTYSIDLPGLASSDSFLQVEDPSNSAVRGAVNDLLAKWHQDYGQVNNVPA
RMQYEKITAHSMEQLKVKFGSDFEKTGNSLDIDFNSVHSGEKQIQIVNFKQIYYTVSVDAVKNPGDVFQDTVTVEDLKQRGIS
AERPLVYISSVAYGRQVYLKLETTSKSDEVEAAFEALIKGVKVAPQTEWKQILDNTEVKAVILCGDPSSGARVVTGKVDMVED
LIQEGSRFTADHPGLPISYTTSFLRDNVVATFQNSADYVETKVTAYRNGDLLLDHSGAYVAQYYITWDELSYDHQGKEVLTPK
AWDRNGQDLTAHFTTSIPLKGNVRNLSVKIREATGLAWEWWRTVYEKTDLPLVRKRTISIWGTTLYPQVEDKVEND
(SEQ ID NO: 29)
SP_Ply_D205R_nIgK
METPAQLLFLLLLWLPDTTGMANKAVNDFILAMNYDKKKLLTHQGESIENRFIKEGNQLPDEFVVIERKKRSLSTNTSDISVT
ATNDSRLYPGALLVVDETLLENNPTLLAVDRAPMTYSIDLPGLASSDSFLQVEDPSNSSVRGAVNDLLAKWHQDYGQVNNVPA
RMQYEKITAHSMEQLKVKFGSDFEKTGNSLDIDFNSVHSGEKQIQIVNFKQIYYTVSVRAVKNPGDVFQDTVTVEDLKQRGIS
AERPLVYISSVAYGRQVYLKLETTSKSDEVEAAFEALIKGVKVAPQTEWKQILDNTEVKAVILGGDPSSGARVVTGKVDMVED
LIQEGSRFTADHPGLPISYTTSFLRDNVVATFQNSTDYVETKVTAYRNGDLLLDHSGAYVAQYYITWDELSYDHQGKEVLTPK
AWDRNGQDLTAHFTTSIPLKGNVRNLSVKIRECTGLAWEWWRTVYEKTDLPLVRKRTISIWGTTLYPQVEDKVEND
(SEQ ID NO: 36)
SP_Ply_L460D_nIgK
METPAQLLFLLLLWLPDTTGMANKAVNDFILAMNYDKKKLLTHQGESIENRFIKEGNQLPDEFVVIERKKRSLSTNTSDISVT
ATNDSRLYPGALLVVDETLLENNPTLLAVDRAPMTYSIDLPGLASSDSFLQVEDPSNSSVRGAVNDLLAKWHQDYGQVNNVPA
RMQYEKITAHSMEQLKVKFGSDFEKTGNSLDIDFNSVHSGEKQIQIVNFKQIYYTVSVDAVKNPGDVFQDTVTVEDLKQRGIS
AERPLVYISSVAYGRQVYLKLETTSKSDEVEAAFEALIKGVKVAPQTEWKQILDNTEVKAVILGGDPSSGARVVTGKVDMVED
LIQEGSRFTADHPGLPISYTTSFLRDNVVATFQNSTDYVETKVTAYRNGDLLLDHSGAYVAQYYITWDELSYDHQGKEVLTPK
AWDRNGQDLTAHFTTSIPLKGNVRNLSVKIRECTGLAWEWWRTVYEKTDLPLVRKRTISIWGTTDYPQVEDKVEND
(SEQ ID NO: 37)
SP_Ply_T65C_G293C_C428A_PlyD1_nIgK
METPAQLLFLLLLWLPDTTGMANKAVNDFILAMNYDKKKLLTHQGESIENRFIKEGNQLPDEFVVIERKKRSLSTNTSDISVT
ACNDSRLYPGALLVVDETLLENNPTLLAVDRAPMTYSIDLPGLASSDSFLQVEDPSNSSVRGAVNDLLAKWHQDYGQVNNVPA
RMQYEKITAHSMEQLKVKFGSDFEKTGNSLDIDFNSVHSGEKQIQIVNFKQIYYTVSVDAVKNPGDVFQDTVTVEDLKQRGIS
AERPLVYISSVAYGRQVYLKLETTSKSDEVEAAFEALIKGVKVAPQTEWKQILDNTEVKAVILCGDPSSGARVVTGKVDMVED
LIQEGSRFTADHPGLPISYTTSFLRDNVVATFQNSTDYVETKVTAYRNGDLLLDHSGAYVAQYYITWDELSYDHQGKEVLTPK
AWDRNGQDLTAHFTTSIPLKGNVRNLSVKIREATGLAWEWWRTVYEKTDLPLVRKRTISIWGTTLYPQVEDKVEND
(SEQ ID NO: 38)

### EQUIVALENTS

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

What is claimed is:

1. A bacterial vaccine, comprising:
at least one RNA polynucleotide having an open reading frame encoding at least one mutated bacterial antigenic polypeptide, wherein the mutated bacterial antigenic polypeptide comprises at least one asparagine (Asn) amino acid of a corresponding wild type bacterial antigenic polypeptide which has been replaced with a non-Asn amino acid.

2. The bacterial vaccine of claim 1, wherein the RNA polynucleotide is formulated in a cationic lipid nanoparticle.

3. The bacterial vaccine of claim 1 or 2, wherein the mutated bacterial antigenic polypeptide has one Asn amino acid of a corresponding wild type bacterial antigenic polypeptide which has been replaced with a non-Asn amino acid.

4. The bacterial vaccine of claim 1 or 2, wherein the mutated bacterial antigenic polypeptide has two Asn amino acids of a corresponding wild type bacterial antigenic polypeptide which have been replaced with a non-Asn amino acid.

5. The bacterial vaccine of claim 1 or 2, wherein the mutated bacterial antigenic polypeptide has three Asn amino acids of a corresponding wild type bacterial antigenic polypeptide which have been replaced with a non-Asn amino acid.

6. The bacterial vaccine of claim 1 or 2, wherein the mutated bacterial antigenic polypeptide has four Asn amino acids of a corresponding wild type bacterial antigenic polypeptide which have been replaced with a non-Asn amino acid.

7. The bacterial vaccine of claim 1 or 2, wherein the mutated bacterial antigenic polypeptide has five Asn amino acids of a corresponding wild type bacterial antigenic polypeptide which have been replaced with a non-Asn amino acid.

8. The bacterial vaccine of any one of claims 1-7, wherein the Asn amino acid has been replaced with a Ala amino acid.

9. The bacterial vaccine of any one of claims 1-8, wherein the mutated bacterial antigenic polypeptide has greater than 80% sequence identity to a wild type bacterial antigenic polypeptide.

10. The bacterial vaccine of any one of claims 1-8, wherein the mutated bacterial antigenic polypeptide has greater than 90% sequence identity to a wild type bacterial antigenic polypeptide.

11. The bacterial vaccine of any one of claims 1-8, wherein the mutated bacterial antigenic polypeptide has greater than 95% sequence identity to a wild type bacterial antigenic polypeptide.

12. The bacterial vaccine of any one of claims 1-8, wherein the mutated bacterial antigenic polypeptide has greater than 98% sequence identity to a wild type bacterial antigenic polypeptide.

13. The bacterial vaccine of any one of claims 1-12, wherein the bacterial vaccine produces a lower IgG titer than an RNA vaccine encoding a corresponding wild type antigen.

14. The bacterial vaccine of any one of claims 1-13, wherein the bacterial vaccine has enhanced neutralization activity relative to an RNA vaccine encoding a corresponding wild type antigen.

15. The bacterial vaccine of any one of claims 1-14, wherein the mutated bacterial antigenic polypeptide is a mutated antigen of an infectious bacteria selected from the group consisting of Streptococcus and Staphylococcus.

16. The bacterial vaccine of claim 15, wherein the Streptococcus is Streptococcus pneumoniae.

17. The bacterial vaccine of claim 15 or 16, wherein the mutated antigen is a pneumolysin.

18. A method of vaccinating a subject, comprising administering the bacterial vaccine of any one of claims 1-17 to the subject in an effective amount to induce an immune response against the bacteria in the subject.

19. The method of claim 17, wherein the immune response is an enhanced neutralization activity relative to an RNA vaccine encoding a corresponding wild type antigen.

20. A Streptococcal vaccine, comprising:
at least one RNA polynucleotide having an open reading frame encoding at least one Streptococcal antigenic polypeptide.

21. The Streptococcal vaccine of claim 20, wherein the Streptococcal antigenic polypeptide is a Streptococcus pneumoniae antigenic polypeptide.

22. The Streptococcal vaccine of claim 20 or 21, wherein the Streptococcal antigenic polypeptide is a pneumolysin.

23. The Streptococcal vaccine of claim 22, wherein the pneumolysin has a wild type pneumolysin sequence.

24. The Streptococcal vaccine of claim 22, wherein the pneumolysin has a modified pneumolysin sequence.

25. The Streptococcal vaccine of claim 24, wherein the modified pneumolysin sequence includes a D205R mutation.

26. The Streptococcal vaccine of any one of claims 20-25, wherein the at least one RNA polynucleotide has a nucleic acid sequence that has at least 80% identity to any one of SEQ ID NO: 6-8, but does not include wild-type mRNA sequence.

27. The Streptococcal vaccine of any one of claims 20-25, wherein the at least one RNA polynucleotide has a nucleic acid sequence that has at least 85% identity to any one of SEQ ID NO: 6-8, but does not include wild-type mRNA sequence.

28. The Streptococcal vaccine of any one of claims 20-25, wherein the at least one RNA polynucleotide has a nucleic acid sequence that has at least 90% identity to any one of SEQ ID NO: 6-8, but does not include wild-type mRNA sequence.

29. The Streptococcal vaccine of any one of claims 20-25, wherein the at least one RNA polynucleotide has a nucleic acid sequence that has at least 95% identity to any one of SEQ ID NO: 6-8, but does not include wild-type mRNA sequence.

30. The Streptococcal vaccine of any one of claims 20-25, wherein the at least one RNA polynucleotide has a nucleic acid sequence that has at least 98% identity to any one of SEQ ID NO: 6-8, but does not include wild-type mRNA sequence.

31. The Streptococcal vaccine of any one of claims 20-30, wherein the Streptococcal antigenic polypeptide has an amino acid sequence that has at least 90% identity to an amino acid sequence identified by any one of SEQ ID NO: 10-29, but does not include wild-type protein sequence.

32. The Streptococcal vaccine of any one of claims 20-30, wherein the Streptococcal antigenic polypeptide has an amino acid sequence that has at least 95% identity to an amino acid sequence identified by any one of SEQ ID NO: 10-29, but does not include wild-type protein sequence.

33. The Streptococcal vaccine of any one of claims 20-30, wherein the Streptococcal antigenic polypeptide has an amino acid sequence that has at least 99% identity to an amino acid sequence identified by any one of SEQ ID NO: 10-29, but does not include wild-type protein sequence.

34. The Streptococcal vaccine of any one of claims 20-30, wherein the Streptococcal antigenic polypeptide has an amino acid sequence of any one of SEQ ID NO: 10-29.

35. The Streptococcal vaccine of any one of claims 20-25, wherein the at least one RNA polynucleotide has a nucleic acid sequence of any one of SEQ ID NO: 6-8.

36. The Streptococcal vaccine of any one of claims 20-35, wherein the RNA polynucleotide is formulated in a cationic lipid nanoparticle.

37. The bacterial or Streptococcal vaccine of any one of claims 1-17 and 20-36, wherein the at least one RNA polynucleotide comprises at least one chemical modification.

38. The bacterial or Streptococcal vaccine of claim 37, wherein the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

39. The bacterial or Streptococcal vaccine of claim 37 or 38, wherein the chemical modification is in the 5-position of the uracil.

40. The bacterial or Streptococcal vaccine of claim 37 or 38, wherein the chemical modification is a N1-methylpseudouridine or N1-ethylpseudouridine.

41. The bacterial or Streptococcal vaccine of claim 37 or 38, wherein at least 80% of the uracil in the open reading frame have a chemical modification.

42. The bacterial or Streptococcal vaccine of claim 37 or 38, wherein at least 90% of the uracil in the open reading frame have a chemical modification.

43. The bacterial or Streptococcal vaccine of claim 37 or 38, wherein 100% of the uracil in the open reading frame have a chemical modification.

44. The bacterial or Streptococcal vaccine of claim 37 or 38, wherein 100% of the uracil in the open reading frame is modified to include N1-methyl pseudouridine at the 5-position of the uracil.

45. The bacterial or Streptococcal vaccine of any one of claims 1-17 and 20-36, wherein at least one RNA polynucleotide further encodes at least one 5′ terminal cap.

46. The bacterial or Streptococcal vaccine of claim 45, wherein the 5′ terminal cap is 7mG(5′)ppp(5′)NlmpNp.

47. The bacterial or Streptococcal vaccine of any one of claims 2-17 and 36-46, wherein the cationic lipid nanoparticle has a mean diameter of 50-200 nm.

48. The bacterial or Streptococcal vaccine of any one of claims 2-17 and 36-46, wherein the cationic lipid nanoparticle comprises a cationic lipid, a PEG-modified lipid, a sterol and a non-cationic lipid.

49. The bacterial or Streptococcal vaccine of any one of claims 2-17 and 36-46, wherein the cationic lipid nanoparticle comprises a molar ratio of about 20-60% cationic lipid, 0.5-15% PEG-modified lipid, 25-55% sterol, and 5-25% non-cationic lipid.

50. The bacterial or Streptococcal vaccine of claim 48 or 49, wherein the cationic lipid is an ionizable cationic lipid and the non-cationic lipid is a neutral lipid, and the sterol is a cholesterol.

51. The bacterial or Streptococcal vaccine of claim 49 or 50, wherein the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

52. The bacterial or Streptococcal vaccine of any one of claims 47-51, wherein the cationic lipid nanoparticle comprises a compound of Formula (I), optionally Compound 3, 18, 20, 25, 26, 29, 30, 60, 108-112, or 122.

53. The bacterial or Streptococcal vaccine of any one of claims 47-51, wherein the cationic lipid nanoparticle comprises a compound of Formula (II).

54. The bacterial or Streptococcal vaccine of any one of claims 47-51, wherein the cationic lipid nanoparticle has a polydispersity value of less than 0.4.

55. The bacterial or Streptococcal vaccine of any one of claims 47-51, wherein the cationic lipid nanoparticle has a net neutral charge at a neutral pH value.

56. The bacterial or Streptococcal vaccine of any one of claims 1-17 and 20-55, further comprising an adjuvant.

57. The bacterial or Streptococcal vaccine of any one of claims 1-17 and 20-56, wherein, wherein the open reading frame is codon-optimized.

58. The bacterial or Streptococcal vaccine of any one of claims 1-17 and 20-57, wherein the vaccine is multivalent.

59. The bacterial or Streptococcal vaccine of any one of claims 1-17 and 20-58, formulated in an effective amount to produce an antigen-specific immune response.

60. The bacterial or Streptococcal vaccine of any one of claims 1-17 and 20-58 for use in a method of inducing an antigen specific immune response in a subject, the method comprising administering to the subject the vaccine in an amount effective to produce an antigen specific immune response in the subject.

61. A pharmaceutical composition for use in vaccination of a subject comprising
an effective dose of a bacterial or Streptococcal vaccine of any one of claims 1-17 and 20-58, wherein the effective dose is sufficient to produce detectable levels of antigen as measured in serum of the subject at 1-72 hours post administration.

62. The composition of claim 61, wherein the cut off index of the antigen is 1-2.

63. A pharmaceutical composition for use in vaccination of a subject comprising
an effective dose of a bacterial or Streptococcal vaccine of any one of claims 1-17 and 20-58, wherein the effective dose is sufficient to produce a 1,000-10,000 neutralization titer produced by neutralizing antibody against said antigen as measured in serum of the subject at 1-72 hours post administration.

64. A composition comprising a bacterial or Streptococcal vaccine of any one of claims 1-17 and 20-58 formulated in a lipid nanoparticle comprising compounds of Formula (I): 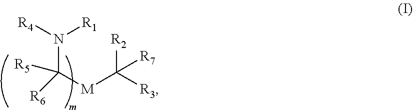
or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)—N(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

65. The vaccine of claim 64, wherein a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

66. The vaccine of claim 64, wherein a subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

67. The vaccine of claim 64, wherein a subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)—N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

68. The vaccine of claim 64, wherein subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

69. The vaccine of claim 64, wherein a subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

70. The vaccine of claim 64, wherein a subset of compounds of Formula (I) includes those of Formula (IA): 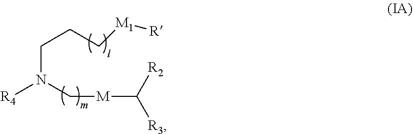
or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

71. A method of inducing an immune response in a subject, the method comprising administering to the subject the Streptococcal vaccine of any one of claims 20-59 in an amount effective to produce an antigen-specific immune response in the subject.

72. The method of claim 71, wherein the antigen specific immune response comprises a T cell response or a B cell response.

73. The method of claim 71 or 72, wherein the subject is administered a single dose of the vaccine.

74. The method of claim 71 or 72, wherein the subject is administered a booster dose of the vaccine.

75. The method of any one of claims 71-74, wherein the vaccine is administered to the subject by intradermal injection or intramuscular injection.

76. The method of any one of claims 71-75, wherein an anti-antigenic polypeptide antibody titer produced in the subject is increased by at least 1 log relative to a control.

77. The method of any one of claims 71-76, wherein an anti-antigenic polypeptide antibody titer produced in the subject is increased by 1-3 log relative to a control.

78. The method of any one of claims 71-77, wherein the anti-antigenic polypeptide antibody titer produced in the subject is increased at least 2 times relative to a control.

79. The method of any one of claims 71-78, wherein the anti-antigenic polypeptide antibody titer produced in the subject is increased 2-10 times relative to a control.

80. The method of any one of claims 76-79, wherein the control is an anti-antigenic polypeptide antibody titer produced in a subject who has not been administered a vaccine against the bacteria.

81. The method of any one of claims 76-79, wherein the control is an anti-antigenic polypeptide antibody titer produced in a subject who has been administered a live attenuated vaccine or an inactivated vaccine against the bacteria.

82. The method of any one of claims 76-79, wherein the control is an anti-antigenic polypeptide antibody titer produced in a subject who has been administered a recombinant protein vaccine or purified protein vaccine against the bacteria.

83. The method of any one of claims 71-82, wherein the effective amount is a dose equivalent to an at least 2-fold reduction in the standard of care dose of a recombinant protein vaccine or a purified protein vaccine against the bacteria, and wherein an anti-antigenic polypeptide antibody titer produced in the subject is equivalent to an anti-antigenic polypeptide antibody titer produced in a control subject administered the standard of care dose of a recombinant protein vaccine or a purified protein vaccine against the bacteria, respectively.

84. The method of any one of claims 71-82, wherein the effective amount is a dose equivalent to an at least 2-fold reduction in the standard of care dose of a live attenuated vaccine or an inactivated vaccine against the bacteria, and wherein an anti-antigenic polypeptide antibody titer produced in the subject is equivalent to an anti-antigenic polypeptide antibody titer produced in a control subject administered the standard of care dose of a live attenuated vaccine or an inactivated vaccine against the bacteria, respectively.

85. The method of any one of claims 71-82, wherein the effective amount is a dose equivalent to an at least 2-fold reduction in the standard of care dose of a adjuvanted peptide vaccine against the bacteria, and wherein an anti-antigenic polypeptide antibody titer produced in the subject is equivalent to an anti-antigenic polypeptide antibody titer produced in a control subject administered the standard of care dose of an adjuvanted peptide vaccine against the bacteria.

86. The method of any one of claims 71-85, wherein the effective amount is a total dose of 50 μg-1000 μg.

87. The method of claim 86, wherein the effective amount is a dose of 25 μg, 100 μg, 400 μg, or 500 μg administered to the subject a total of two times.

88. The method of any one of claims 71-87, wherein the efficacy of the vaccine against the bacteria is greater than 65%.

89. The method of any one of claims 71-88, wherein the vaccine immunizes the subject against the bacteria for up to 2 years.

90. The method of any one of claims 71-89, wherein the vaccine immunizes the subject against the bacteria for more than 2 years.

91. The method of any one of claims 71-90, wherein the subject has an age of about 12 to about 50 years old.

92. The method of any one of claims 71-91, wherein the subject has been exposed to the bacteria, wherein the subject is infected with the bacteria, or wherein the subject is at risk of infection by the bacteria.

93. The method of any one of claims 71-92, wherein the subject is immunocompromised.


SEQUENCE LISTING (69 sequences)

SEQ ID NO: 1 - DNA, 1649 bp, S.pneumoniae
ttacaagaccaaccttgattgacttagataaggtatttatgttggataatacggttattccgacttcttatctagccagacggcgacgcaatgtctcagaagaattgtacgaggaaattttggatcacttagtccaaccacggctgatttcgctgaacaagtctgagtttatgcaactcaatccaggaacttattaggaggagaagatggcaaataaagcagtaaatgactttatactagctatgaattacgataaaaagaaactcttgacccatcagggagaaagtattgaaaatcgtttcatcaaagagggtaatcagctacccgatgagtttgttgttatcgaaagaaagaagcggagcttgtcgacaaatacaagtgatatttctgtaacagctaccaacgacagtcgcctctatcctggagcacttctcgtagtggatgagaccttgttagagaataatcccactcttcttgcggttgatcgtgctccgatgacttatagtattgatttgcctggtttggcaagtagcgatagctttctccaagtggaagaccccagcaattcaagtgttcgcggagcggtaaacgatttgttggctaagtggcatcaagattatggtcaggtcaataatgtcccagctagaatgcagtatgaaaaaataacggctcacagcatggaacaactcaaggtcaagtttggttctgactttgaaaagacagggaattctcttgatattgattttaactctgtccattcaggtgaaaagcagattcagattgttaattttaagcagatttattatacagtcagcgtagacgctgttaaaaatccaggagatgtgtttcaagatactgtaacggtagaggatttaaaacagagaggaatttctgcagagcgtcctttggtctatatttcgagtgttgcttatgggcgccaagtctatctcaagttggaaaccacgagtaagagtgatgaagtagaggctgcttttgaagctttgataaaaggagtcaaggtagctcctcagacagagtggaagcagattttggacaatacagaagtgaaggcggttattttagggggcgacccaagttcgggtgcccgagttgtaacaggcaaggtggatatggtagaggacttgattcaagaaggcagtcgctttacagcagatcatccaggcttgccgatttcctatacaacttcttttttacgtgacaatgtagttgcgacctttcaaaacagtacagactatgttgagactaaggttacagcttacagaaacggagatttactgctggatcatagtggtgcctatgttgcccaatattatattacttgggatgaattatcctatgatcatcaaggtaaggaagtcttgactcctaaggcttgggacagaaatgggcaggatttgacggctcactttaccactagtattcctttaaaagggaatgttcgtaatctctctgtcaaaattagagagtgtaccgggcttgcctgggaatggtggcgtacggtttatgaaaaaaccgatttgccactagtgcgtaagcggacgatttctatttggggaacaactctctatcctcaggtagaggataaggtagaaaatgactaggagaggagaatgcttgcgacaaaaaga

SEQ ID NO: 2 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgccaccaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtgcgggccgtgaagaaccctggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgaccgcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccacgctgtatcctcaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 3 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgccaccaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgaccgcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccaccgactacccccaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 4 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgcctgcaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgtgcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgacagcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagaagccaccggactggcctgggagtggtggcggacagtgtacgaaaagaccgacctgcccctcgtgcggaagcggaccatctctatctggggcaccacgctgtatcctcaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 5 - RNA, 1649 nt, S.pneumoniae
uuacaagaccaaccuugauugacuuagauaagguauuuauguuggauaauacgguuauuccgacuucuuaucuagccagacggcgacgcaaugucucagaagaauuguacgaggaaauuuuggaucacuuaguccaaccacggcugauuucgcugaacaagucugaguuuaugcaacucaauccaggaacuuauuaggaggagaagauggcaaauaaagcaguaaaugacuuuauacuagcuaugaauuacgauaaaaagaaacucuugacccaucagggagaaaguauugaaaaucguuucaucaaagaggguaaucagcuacccgaugaguuuguuguuaucgaaagaaagaagcggagcuugucgacaaauacaagugauauuucuguaacagcuaccaacgacagucgccucuauccuggagcacuucucguaguggaugagaccuuguuagagaauaaucccacucuucuugcgguugaucgugcuccgaugacuuauaguauugauuugccugguuuggcaaguagcgauagcuuucuccaaguggaagaccccagcaauucaaguguucgcggagcgguaaacgauuuguuggcuaaguggcaucaagauuauggucaggucaauaaugucccagcuagaaugcaguaugaaaaaauaacggcucacagcauggaacaacucaaggucaaguuugguucugacuuugaaaagacagggaauucucuugauauugauuuuaacucuguccauucaggugaaaagcagauucagauuguuaauuuuaagcagauuuauuauacagucagcguagacgcuguuaaaaauccaggagauguguuucaagauacuguaacgguagaggauuuaaaacagagaggaauuucugcagagcguccuuuggucuauauuucgaguguugcuuaugggcgccaagucuaucucaaguuggaaaccacgaguaagagugaugaaguagaggcugcuuuugaagcuuugauaaaaggagucaagguagcuccucagacagaguggaagcagauuuuggacaauacagaagugaaggcgguuauuuuagggggcgacccaaguucgggugcccgaguuguaacaggcaagguggauaugguagaggacuugauucaagaaggcagucgcuuuacagcagaucauccaggcuugccgauuuccuauacaacuucuuuuuuacgugacaauguaguugcgaccuuucaaaacaguacagacuauguugagacuaagguuacagcuuacagaaacggagauuuacugcuggaucauaguggugccuauguugcccaauauuauauuacuugggaugaauuauccuaugaucaucaagguaaggaagucuugacuccuaaggcuugggacagaaaugggcaggauuugacggcucacuuuaccacuaguauuccuuuaaaagggaauguucguaaucucucugucaaaauuagagaguguaccgggcuugccugggaaugguggcguacgguuuaugaaaaaaccgauuugccacuagugcguaagcggacgauuucuauuuggggaacaacucucuauccucagguagaggauaagguagaaaaugacuaggagaggagaaugcuugcgacaaaaaga

SEQ ID NO: 6 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccaccaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccgugcgggccgugaagaacccuggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugaccgccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccacgcuguauccucagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 7 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccaccaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugaccgccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccaccgacuacccccagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 8 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccugcaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugugcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugacagccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagaagccaccggacuggccugggagugguggcggacaguguacgaaaagaccgaccugccccucgugcggaagcggaccaucucuaucuggggcaccacgcuguauccucagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 9 - protein, 471 aa, S.pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 10 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 11 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIFLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 12 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIMLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 13 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLMSSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 14 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLQSSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 15 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSFSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 16 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGRKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 17 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGLKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 18 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIFLPGLQSSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 19 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIFLPGLASSDSFLQVEDPSFSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 20 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLQSSDSFLQVEDPSFSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 21 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIFLPGLQSSDSFLQVEDPSFSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 22 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIFLPGLQSSDSFLQVEDPSFSSVRGAVNDLLAKWHQDYGQVNNVPARMQYEKITAHSMEQLKVKFGSDFEKTGNSLDIDFNSVHSGLKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 23 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVFAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 24 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVPAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 25 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVRNPGDVFQDTVTVEDLKQRGISAERPLVYISSVAYGRQVYLKLETTSKSDEVEAAFEALIKGVKVAPQTEWKQILDNTEVKAVILIGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 26 - protein, 477 aa, Artificial Sequence
  Synthetic polypeptide
HHHHHHGSANKAVNDFILAMNYDKKKLLTHQGESIENRFIKEGNQLPDEFVVIERKKRSLSTSTSDISVTATNDSRLYPGALLVVDETLLENNPTLLAVDRAPMTYSIDLPGLASSDSFLQVEDPSFSSVRGAVNDLLAKWHQDYGQVNNVPARMQYEKITAHSMEQLKVKFGSDFEKTGNSLDIDFNSVHSGEKQIQIVNFKQIYYTVSVPAVRNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVDDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEN

SEQ ID NO: 27 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTDTSDISVTATNDARLYPGALLVVDETLLENNPILLAVDRAPMTYSIDLPGLASSDSFLQVEDPSNSAVRGAVNDLLAKWHQDYGQVNNVPARMQYEKITAHSMEQLKVKFGSDFEKTGNSLDIDFNSVHSGEKQIQIVNFKQIYYTVSVRAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSADYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 28 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTDTSDISVTATNDARLYPGALLVVDETLLENNPILLAVDRAPMTYSIDLPGLASSDSFLQVEDPSNSAVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSADYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVEND

SEQ ID NO: 29 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTDTSDISVTACNDARLYPGALLVVDETLLENNPILLAVDRAPMTYSIDLPGLASSDSFLQVEDPSNSAVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSADYVETKVTAYRNGDLLLDHSGAYVAQYYITWDELSYDHQGKEVLTPKAWDRNGQDLTAHFTTSIPLKGNVRNLSVKIREATGLAWEWWRTVYEKTDLPLVRKRTISIWGTTLYPQVEDKVEND

SEQ ID NO: 30 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgccaccaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtgcgggccgtgaagaaccctggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgaccgcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccacgctgtatcctcaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 31 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgccaccaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgaccgcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccaccgattacccccaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 32 - DNA, 1684 bp, Artificial Sequence
  Synthetic polynucleotide
tcaagcttttggaccctcgtacagaagctaatacgactcactatagggaaataagagagaaaagaagagtaagaagaaatataagagccaccatggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgcctgcaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgtgcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgacagcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagaagccaccggactggcctgggagtggtggcggacagtgtacgaaaagaccgacctgcccctcgtgcggaagcggaccatctctatctggggcaccacgctgtatcctcaggtggaagataaggtggaaaacgactgataataggctggagcctcggtggccatgcttcttgccccttgggcctccccccagcccctcctccccttcctgcacccgtacccccgtggtctttgaataaagtctgagtgggcggc

SEQ ID NO: 33 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccaccaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccgugcgggccgugaagaacccuggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugaccgccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccacgcuguauccucagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 34 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccaccaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugaccgccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccaccgauuacccccagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 35 - RNA, 1684 nt, Artificial Sequence
  Synthetic polynucleotide
ucaagcuuuuggacccucguacagaagcuaauacgacucacuauagggaaauaagagagaaaagaagaguaagaagaaauauaagagccaccauggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccugcaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugugcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugacagccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagaagccaccggacuggccugggagugguggcggacaguguacgaaaagaccgaccugccccucgugcggaagcggaccaucucuaucuggggcaccacgcuguauccucagguggaagauaagguggaaaacgacugauaauaggcuggagccucgguggccaugcuucuugccccuugggccuccccccagccccuccuccccuuccugcacccguacccccguggucuuugaauaaagucugagugggcggc

SEQ ID NO: 36 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVRAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 37 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTDYPQVEDKVEND

SEQ ID NO: 38 - protein, 491 aa, Artificial Sequence
  Synthetic polypeptide
METPAQLLFLLLLWLPDTTGMANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIREATGLAWEWWRTVYEKTDLPLVRKRTISIWGTTLYPQVEDKVEND

SEQ ID NO: 39 - protein, 20 aa, Artificial Sequence
  Synthetic polypeptide
METPAQLLFLLLLWLPDTTG

SEQ ID NO: 40 - protein, 18 aa, Artificial Sequence
  Synthetic polypeptide
MDWTWILFLVAAATRVHS

SEQ ID NO: 41 - protein, 24 aa, Japanese encephalitis
MLGSNSGQRVVFTILLLLVAPAYS

SEQ ID NO: 42 - protein, 17 aa, Artificial Sequence
  Synthetic polypeptide
MKCLLYLAFLFIGVNCA

SEQ ID NO: 43 - protein, 15 aa, Japanese encephalitis
MWLVSLAIVTACAGA

SEQ ID NO: 44 - DNA, 1729 bp, Artificial Sequence
  Synthetic polynucleotide
tcaagcttttggaccctcgtacagaagctaatacgactcactatagggaaataagagagaaaagaagagtaagaagaaatataagagccaccatggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgttgataataggctggagcctcggtggccatgcttcttgccccttgggcctccccccagcccctcctccccttcctgcacccgtacccccgtggtctttgaataaagtctgagtgggcggc

SEQ ID NO: 45 - DNA, 1518 bp, Artificial Sequence
  Synthetic polynucleotide
atggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgt

SEQ ID NO: 46 - RNA, 1790 nt, Artificial Sequence
  Synthetic polynucleotide
ggggaaauaagagagaaaagaagaguaagaagaaauauaagagccaccauggcacaagucauuaauacaaacagccugucgcuguugacccagaauaaccugaacaaaucccaguccgcacugggcacugcuaucgagcguuugucuuccggucugcguaucaacagcgcgaaagacgaugcggcaggacaggcgauugcuaaccguuuuaccgcgaacaucaaaggucugacucaggcuucccguaacgcuaacgacgguaucuccauugcgcagaccacugaaggcgcgcugaacgaaaucaacaacaaccugcagcgugugcgugaacuggcgguucagucugcgaaugguacuaacucccagucugaccucgacuccauccaggcugaaaucacccagcgccugaacgaaaucgaccguguauccggccagacucaguucaacggcgugaaaguccuggcgcaggacaacacccugaccauccagguuggugccaacgacggugaaacuaucgauauugauuuaaaagaaaucagcucuaaaacacugggacuugauaagcuuaauguccaagaugccuacaccccgaaagaaacugcuguaaccguugauaaaacuaccuauaaaaaugguacagauccuauuacagcccagagcaauacugauauccaaacugcaauuggcgguggugcaacggggguuacuggggcugauaucaaauuuaaagauggucaauacuauuuagauguuaaaggcggugcuucugcugguguuuauaaagccacuuaugaugaaacuacaaagaaaguuaauauugauacgacugauaaaacuccguuggcaacugcggaagcuacagcuauucggggaacggccacuauaacccacaaccaaauugcugaaguaacaaaagaggguguugauacgaccacaguugcggcucaacuugcugcagcagggguuacuggcgccgauaaggacaauacuagccuuguaaaacuaucguuugaggauaaaaacgguaagguuauugaugguggcuaugcagugaaaaugggcgacgauuucuaugccgcuacauaugaugagaaaacaggugcaauuacugcuaaaaccacuacuuauacagaugguacuggcguugcucaaacuggagcugugaaauuugguggcgcaaaugguaaaucugaaguuguuacugcuaccgaugguaagacuuacuuagcaagcgaccuugacaaacauaacuucagaacaggcggugagcuuaaagagguuaauacagauaagacugaaaacccacugcagaaaauugaugcugccuuggcacagguugauacacuucguucugaccugggugcgguucagaaccguuucaacuccgcuaucaccaaccugggcaauaccguaaauaaccugucuucugcccguagccguaucgaagauuccgacuacgcaaccgaagucuccaacaugucucgcgcgcagauucugcagcaggccgguaccuccguucuggcgcaggcgaaccagguuccgcaaaacguccucucuuuacugcguugauaauaggcuggagccucgguggccaugcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 47 - protein, 506 aa, Artificial Sequence
  Synthetic polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 48 - protein, 698 aa, Artificial Sequence
  Synthetic polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRGGGGSGGGGSMMAPDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIKNSISTEWSPCSVTCGNGIQVRIKPGSANKPKDELDYENDIEKKICKMEKCSSVFNVVNS

SEQ ID NO: 49 - protein, 692 aa, Artificial Sequence
  Synthetic polypeptide
MMAPDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIKNSISTEWSPCSVTCGNGIQVRIKPGSANKPKDELDYENDIEKKICKMEKCSSVFNVVNSRPVTMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 50 - RNA, 1729 nt, Artificial Sequence
  Synthetic polynucleotide
ucaagcuuuuggacccucguacagaagcuaauacgacucacuauagggaaauaagagagaaaagaagaguaagaagaaauauaagagccaccauggcacaagucauuaauacaaacagccugucgcuguugacccagaauaaccugaacaaaucccaguccgcacugggcacugcuaucgagcguuugucuuccggucugcguaucaacagcgcgaaagacgaugcggcaggacaggcgauugcuaaccguuuuaccgcgaacaucaaaggucugacucaggcuucccguaacgcuaacgacgguaucuccauugcgcagaccacugaaggcgcgcugaacgaaaucaacaacaaccugcagcgugugcgugaacuggcgguucagucugcgaaugguacuaacucccagucugaccucgacuccauccaggcugaaaucacccagcgccugaacgaaaucgaccguguauccggccagacucaguucaacggcgugaaaguccuggcgcaggacaacacccugaccauccagguuggugccaacgacggugaaacuaucgauauugauuuaaaagaaaucagcucuaaaacacugggacuugauaagcuuaauguccaagaugccuacaccccgaaagaaacugcuguaaccguugauaaaacuaccuauaaaaaugguacagauccuauuacagcccagagcaauacugauauccaaacugcaauuggcgguggugcaacggggguuacuggggcugauaucaaauuuaaagauggucaauacuauuuagauguuaaaggcggugcuucugcugguguuuauaaagccacuuaugaugaaacuacaaagaaaguuaauauugauacgacugauaaaacuccguuggcaacugcggaagcuacagcuauucggggaacggccacuauaacccacaaccaaauugcugaaguaacaaaagaggguguugauacgaccacaguugcggcucaacuugcugcagcagggguuacuggcgccgauaaggacaauacuagccuuguaaaacuaucguuugaggauaaaaacgguaagguuauugaugguggcuaugcagugaaaaugggcgacgauuucuaugccgcuacauaugaugagaaaacaggugcaauuacugcuaaaaccacuacuuauacagaugguacuggcguugcucaaacuggagcugugaaauuugguggcgcaaaugguaaaucugaaguuguuacugcuaccgaugguaagacuuacuuagcaagcgaccuugacaaacauaacuucagaacaggcggugagcuuaaagagguuaauacagauaagacugaaaacccacugcagaaaauugaugcugccuuggcacagguugauacacuucguucugaccugggugcgguucagaaccguuucaacuccgcuaucaccaaccugggcaauaccguaaauaaccugucuucugcccguagccguaucgaagauuccgacuacgcaaccgaagucuccaacaugucucgcgcgcagauucugcagcaggccgguaccuccguucuggcgcaggcgaaccagguuccgcaaaacguccucucuuuacugcguugauaauaggcuggagccucgguggccaugcuucuugccccuugggccuccccccagccccuccuccccuuccugcacccguacccccguggucuuugaauaaagucugagugggcggc

SEQ ID NO: 51 - RNA, 1518 nt, Artificial Sequence
  Synthetic polynucleotide
auggcacaagucauuaauacaaacagccugucgcuguugacccagaauaaccugaacaaaucccaguccgcacugggcacugcuaucgagcguuugucuuccggucugcguaucaacagcgcgaaagacgaugcggcaggacaggcgauugcuaaccguuuuaccgcgaacaucaaaggucugacucaggcuucccguaacgcuaacgacgguaucuccauugcgcagaccacugaaggcgcgcugaacgaaaucaacaacaaccugcagcgugugcgugaacuggcgguucagucugcgaaugguacuaacucccagucugaccucgacuccauccaggcugaaaucacccagcgccugaacgaaaucgaccguguauccggccagacucaguucaacggcgugaaaguccuggcgcaggacaacacccugaccauccagguuggugccaacgacggugaaacuaucgauauugauuuaaaagaaaucagcucuaaaacacugggacuugauaagcuuaauguccaagaugccuacaccccgaaagaaacugcuguaaccguugauaaaacuaccuauaaaaaugguacagauccuauuacagcccagagcaauacugauauccaaacugcaauuggcgguggugcaacggggguuacuggggcugauaucaaauuuaaagauggucaauacuauuuagauguuaaaggcggugcuucugcugguguuuauaaagccacuuaugaugaaacuacaaagaaaguuaauauugauacgacugauaaaacuccguuggcaacugcggaagcuacagcuauucggggaacggccacuauaacccacaaccaaauugcugaaguaacaaaagaggguguugauacgaccacaguugcggcucaacuugcugcagcagggguuacuggcgccgauaaggacaauacuagccuuguaaaacuaucguuugaggauaaaaacgguaagguuauugaugguggcuaugcagugaaaaugggcgacgauuucuaugccgcuacauaugaugagaaaacaggugcaauuacugcuaaaaccacuacuuauacagaugguacuggcguugcucaaacuggagcugugaaauuugguggcgcaaaugguaaaucugaaguuguuacugcuaccgaugguaagacuuacuuagcaagcgaccuugacaaacauaacuucagaacaggcggugagcuuaaagagguuaauacagauaagacugaaaacccacugcagaaaauugaugcugccuuggcacagguugauacacuucguucugaccugggugcgguucagaaccguuucaacuccgcuaucaccaaccugggcaauaccguaaauaaccugucuucugcccguagccguaucgaagauuccgacuacgcaaccgaagucuccaacaugucucgcgcgcagauucugcagcaggccgguaccuccguucuggcgcaggcgaaccagguuccgcaaaacguccucucuuuacugcgu

SEQ ID NO: 52 - RNA, 1790 nt, Artificial Sequence; identical to SEQ ID NO: 46
  Synthetic polynucleotide

SEQ ID NO: 53 - protein, 13 aa, Artificial Sequence
  Synthetic polypeptide
LQRVRELAVQSAN

SEQ ID NO: 54 - RNA, 47 nt, Artificial Sequence
  Synthetic polynycleotide
gggaaauaagagagaaaagaagaguaagaagaaauauaagagccacc

SEQ ID NO: 55 - RNA, 119 nt, Artificial Sequence
  Synthetic polynucleotide
ugauaauaggcuggagccucgguggccuagcuucuugccccuugggccuccccccagccccuccuccccuuccugcacccguacccccguggucuuugaauaaagucugagugggcggc

SEQ ID NO: 56 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgccaccaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtgcgggccgtgaagaaccctggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgaccgcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccacgctgtatcctcaggtggaagataaggtggaaaacgac

SEQ ID NO: 57 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgccaccaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgaccgcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccaccgactacccccaggtggaagataaggtggaaaacgac

SEQ ID NO: 58 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccgacaccagcgacatcagcgtgaccgcctgcaacgacgccagactgtatcctggcgctctgctggtggtggacgagacactgctggaaaacaaccccatcctgctggccgtggacagagcccccatgacctacagcatcgacctgcctggcctggccagcagcgatagctttctgcaggtggaagatcccagcaacagcgccgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagcagcgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgtgcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaactccgccgactacgtggaaacaaaagtgacagcctaccggaacggcgatctgctgctggatcactccggcgcctatgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagaagccaccggactggcctgggagtggtggcggacagtgtacgaaaagaccgacctgcccctcgtgcggaagcggaccatctctatctggggcaccacgctgtatcctcaggtggaagataaggtggaaaacgac

SEQ ID NO: 59 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgccaccaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtgcgggccgtgaagaaccctggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgaccgcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccacgctgtatcctcaggtggaagataaggtggaaaacgac

SEQ ID NO: 60 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgccaccaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgggcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgaccgcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagagtgcaccggactggcctgggagtggtggcggaccgtgtacgaaaagaccgacctgcccctcgtgcggaagcggaccatctctatctggggcaccaccgattacccccaggtggaagataaggtggaaaacgac

SEQ ID NO: 61 - DNA, 1473 bp, Artificial Sequence
  Synthetic polynucleotide
atggaaacccctgcccagctgctgttcctgctgctgctgtggctgcctgacaccaccggcatggccaacaaggccgtgaacgacttcatcctggccatgaactacgacaagaagaagctgctgacccaccagggcgagagcatcgagaacagattcatcaaagagggcaaccagctgcccgacgagttcgtcgtgatcgagcggaagaagcggagcctgagcaccaacaccagcgacatcagcgtgaccgcctgcaacgacagcagactgtatcctggcgccctgctggtggtggacgagacactgctggaaaacaaccccaccctgctggccgtggacagagcccctatgacctacagcatcgacctgcctggcctggccagcagcgatagctttctgcaggtggaagatcccagcaacagcagcgtgcggggagccgtgaatgacctgctggctaagtggcaccaggactacggccaagtgaacaacgtgcccgccagaatgcagtacgagaagatcaccgcccactccatggaacagctgaaagtgaagttcggcagcgacttcgagaaaaccggcaacagcctggacatcgacttcaacagcgtgcacagcggcgagaagcagatccagatcgtgaacttcaagcagatctactacaccgtgtccgtggacgccgtgaagaaccccggggacgtgttccaggataccgtgaccgtggaagatctgaagcagcggggcatcagcgccgagaggccactggtgtacatcagctctgtggcctacggcagacaggtgtacctgaagctggaaaccacctccaagagcgacgaggtggaagccgccttcgaggccctgatcaagggcgtgaaagtggcccctcagaccgagtggaagcagattctggacaacaccgaagtgaaagccgtgatcctgtgcggcgacccttctagcggagccagagtcgtgacaggcaaggtggacatggtggaagatctgatccaggaaggcagccggttcaccgccgatcaccctggcctgcctatcagctacaccacaagctttctgagagacaacgtggtggccacattccagaacagcaccgactacgtggaaacaaaagtgacagcctaccggaacggcgatctgctgctggatcactccggcgcctacgtggcccagtactacatcacctgggacgagctgagctacgatcaccagggcaaagaggtgctgacccccaaggcctgggacagaaacggccaggatctgacagcccacttcacaaccagcatccccctgaagggcaacgtgcggaacctgagcgtgaagatcagagaagccaccggactggcctgggagtggtggcggacagtgtacgaaaagaccgacctgcccctcgtgcggaagcggaccatctctatctggggcaccacgctgtatcctcaggtggaagataaggtggaaaacgac

SEQ ID NO: 62 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccaccaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccgugcgggccgugaagaacccuggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugaccgccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccacgcuguauccucagguggaagauaagguggaaaacgac

SEQ ID NO: 63 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccaccaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugaccgccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccaccgacuacccccagguggaagauaagguggaaaacgac

SEQ ID NO: 64 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccgacaccagcgacaucagcgugaccgccugcaacgacgccagacuguauccuggcgcucugcuggugguggacgagacacugcuggaaaacaaccccauccugcuggccguggacagagcccccaugaccuacagcaucgaccugccuggccuggccagcagcgauagcuuucugcagguggaagaucccagcaacagcgccgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcagcguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugugcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacuccgccgacuacguggaaacaaaagugacagccuaccggaacggcgaucugcugcuggaucacuccggcgccuauguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagaagccaccggacuggccugggagugguggcggacaguguacgaaaagaccgaccugccccucgugcggaagcggaccaucucuaucuggggcaccacgcuguauccucagguggaagauaagguggaaaacgac

SEQ ID NO: 65 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccaccaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccgugcgggccgugaagaacccuggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugaccgccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccacgcuguauccucagguggaagauaagguggaaaacgac

SEQ ID NO: 66 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccaccaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugggcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugaccgccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagagugcaccggacuggccugggagugguggcggaccguguacgaaaagaccgaccugccccucgugcggaagcggaccaucucuaucuggggcaccaccgauuacccccagguggaagauaagguggaaaacgac

SEQ ID NO: 67 - RNA, 1473 nt, Artificial Sequence
  Synthetic polynucleotide
auggaaaccccugcccagcugcuguuccugcugcugcuguggcugccugacaccaccggcauggccaacaaggccgugaacgacuucauccuggccaugaacuacgacaagaagaagcugcugacccaccagggcgagagcaucgagaacagauucaucaaagagggcaaccagcugcccgacgaguucgucgugaucgagcggaagaagcggagccugagcaccaacaccagcgacaucagcgugaccgccugcaacgacagcagacuguauccuggcgcccugcuggugguggacgagacacugcuggaaaacaaccccacccugcuggccguggacagagccccuaugaccuacagcaucgaccugccuggccuggccagcagcgauagcuuucugcagguggaagaucccagcaacagcagcgugcggggagccgugaaugaccugcuggcuaaguggcaccaggacuacggccaagugaacaacgugcccgccagaaugcaguacgagaagaucaccgcccacuccauggaacagcugaaagugaaguucggcagcgacuucgagaaaaccggcaacagccuggacaucgacuucaacagcgugcacagcggcgagaagcagauccagaucgugaacuucaagcagaucuacuacaccguguccguggacgccgugaagaaccccggggacguguuccaggauaccgugaccguggaagaucugaagcagcggggcaucagcgccgagaggccacugguguacaucagcucuguggccuacggcagacagguguaccugaagcuggaaaccaccuccaagagcgacgagguggaagccgccuucgaggcccugaucaagggcgugaaaguggccccucagaccgaguggaagcagauucuggacaacaccgaagugaaagccgugauccugugcggcgacccuucuagcggagccagagucgugacaggcaagguggacaugguggaagaucugauccaggaaggcagccgguucaccgccgaucacccuggccugccuaucagcuacaccacaagcuuucugagagacaacgugguggccacauuccagaacagcaccgacuacguggaaacaaaagugacagccuaccggaacggcgaucugcugcuggaucacuccggcgccuacguggcccaguacuacaucaccugggacgagcugagcuacgaucaccagggcaaagaggugcugacccccaaggccugggacagaaacggccaggaucugacagcccacuucacaaccagcaucccccugaagggcaacgugcggaaccugagcgugaagaucagagaagccaccggacuggccugggagugguggcggacaguguacgaaaagaccgaccugccccucgugcggaagcggaccaucucuaucuggggcaccacgcuguauccucagguggaagauaagguggaaaacgac

SEQ ID NO: 68 - RNA, 11 nt, Artificial Sequence
  Synthetic polynucleotide
gggauccuacc

SEQ ID NO: 69 - RNA, 92 nt, Artificial Sequence
  Synthetic polynucleotide
ucaagcuuuuggacccucguacagaagcuaauacgacucacuauagggaaauaagagagaaaagaagaguaagaagaaauauaagagccacc